SEQUENCE LISTING (170 sequences)

SEQ ID NO: 1 - protein, 280 aa, Homo sapiens
MSAQESCLSLIKYFLFVFNLFFFVLGSLIFCFGIWILIDKTSFVSFVGLAFVPLQIWSKVLAISGIFTMGIALLGCVGALKELRCLLGLYFGMLLLLFATQITLGILISTQRAQLERSLRDVVEKTIQKYGTNPEETAAEESWDYVQFQLRCCGWHYPQDWFQVLILRGNGSEAHRVPCSCYNLSATNDSTILDKVILPQLSRLGHLARSRHSADICAVPAESHIYREGCAQGLQKWLHNNLISIVGICLGVGLLELGFMTLSIFLCRNLDHVYNRLAYR

SEQ ID NO: 2 - DNA, 446 bp, Artificial Sequence
  huCD37-50
aagcttgccaccatggggtggtcctgcataatccttttcctggttgctactgctaccggagtccattcacaggtgcagctgcaggagtccggccccggcctgctcaagccttctcagagtctgagtctgacttgtactgtttctggctacagcataaccagcggtttcgcttggcactggatcagacagcatcccggcaacaaactggagtggatgggatacatactgtactcaggctcaactgtctattccccctccctgaaatcccggatcagtattacccgtgacacttctaagaaccatttttttctgcagctgaacagcgttaccgcagctgacactgcaacctactactgtgcccggggatattatggatacggagcttggttcgcttactggggccaaggcaccctcgtaactgtgagtgctgcttccaccaagggccc

SEQ ID NO: 3 - DNA, 446 bp, Artificial Sequence
  huCD37-51
aagcttgccaccatgggttggtcttgcatcatcctgttcctggtggccactgccactggcgtgcattcagaagttcagttggtggagtccggcccagaagtgctgaaacccggcgaatcactgtccctgacttgtaccgtgtcaggttatagcatcagcagcggctttgcttggcactggattcggcagtttccaggcaagggactggaatggatgggctacatccattacagtggctcaaccaattacagccctagcctgcagggccgaatctctattaccagggatagttctattaaccagtttttcctgcagcttaattccgtgactgcctctgacacagcaacttactattgcgcccgtggctactacgggttcggagcctggtttgtatactggggtcagggcaccctggtcactgtctcagccgcctctaccaagggccc

SEQ ID NO: 4 - protein, 5 aa, Artificial Sequence
  CD37-3 VH-CDR1
TSGVS

SEQ ID NO: 5 - protein, 9 aa, Artificial Sequence
  CD37-3 VH-CDR2
VIWGDGSTN

SEQ ID NO: 6 - protein, 7 aa, Artificial Sequence
  CD37-3 VH-CDR3
GGYSLAH

SEQ ID NO: 7 - protein, 5 aa, Artificial Sequence
  CD37-12 VH-CDR1
KYGMN

SEQ ID NO: 8 - protein, 10 aa, Artificial Sequence
  CD37-12 VH-CDR2
WINTNTGESR

SEQ ID NO: 9 - protein, 6 aa, Artificial Sequence
  CD37-12 VH-CDR3
GTVVAD

SEQ ID NO: 10 - protein, 6 aa, Artificial Sequence
  CD37-38 VH-CDR1
SGFGWH

SEQ ID NO: 11 - protein, 9 aa, Artificial Sequence
  CD37-38 VH-CDR2
YILYSGGTD

SEQ ID NO: 12 - protein, 11 aa, Artificial Sequence
  CD37-38 VH-CDR3
GYYGYGAWFVY

SEQ ID NO: 13 - protein, 6 aa, Artificial Sequence
  CD37-50 VH-CDR1
SGFAWH

SEQ ID NO: 14 - protein, 9 aa, Artificial Sequence
  CD37-50  VH-CDR2
YILYSGSTV

SEQ ID NO: 15 - protein, 11 aa, Artificial Sequence
  CD37-50 VH-CDR3
GYYGYGAWFAY

SEQ ID NO: 16 - protein, 6 aa, Artificial Sequence
  CD37-51 VH-CDR1
SGFAWH

SEQ ID NO: 17 - protein, 9 aa, Artificial Sequence
  CD37-51 VH-CDR2
YIHYSGSTN

SEQ ID NO: 18 - protein, 11 aa, Artificial Sequence
  CD37-51 VH-CDR3
GYYGFGAWFVY

SEQ ID NO: 19 - protein, 6 aa, Artificial Sequence
  CD37-56 VH-CDR1
SGFAWH

SEQ ID NO: 20 - protein, 9 aa, Artificial Sequence
  CD37-56 VH-CDR2
YIHYSGGTN

SEQ ID NO: 21 - protein, 11 aa, Artificial Sequence
  CD37-56 VH-CDR3
GYYGFGAWFAY

SEQ ID NO: 22 - protein, 6 aa, Artificial Sequence
  CD37-57 VH-CDR1
SGFAWH

SEQ ID NO: 23 - protein, 9 aa, Artificial Sequence
  CD37-57 VH-CDR2
YILYSGSTV

SEQ ID NO: 24 - protein, 11 aa, Artificial Sequence
  CD37-57 VH-CDR3
GYYGYGAWFAY

SEQ ID NO: 25 - protein, 6 aa, Artificial Sequence
  CONSENSUS VH-CDR1
SGFXWH

SEQ ID NO: 26 - protein, 8 aa, Artificial Sequence
  CONSENSUS VH-CDR2
YIXYSGTX

SEQ ID NO: 27 - protein, 9 aa, Artificial Sequence
  CONSENSUS VH-CDR3
GYYGGAWFY

SEQ ID NO: 28 - protein, 11 aa, Artificial Sequence
  CD37-3 VL-CDR1
RASENIRSNLA

SEQ ID NO: 29 - protein, 7 aa, Artificial Sequence
  CD37-3 VL-CDR2
VATNLAD

SEQ ID NO: 30 - protein, 9 aa, Artificial Sequence
  CD37-3 VL-CDR3
QHYWGTTWT

SEQ ID NO: 31 - protein, 15 aa, Artificial Sequence
  CD37-12 VL-CDR1
RASQSVSTSSYSYLY

SEQ ID NO: 32 - protein, 7 aa, Artificial Sequence
  CD37-12 VL-CDR1
YASNLAS

SEQ ID NO: 33 - protein, 9 aa, Artificial Sequence
  CD37-12 VL-CDR2
QHSWEIPYT

SEQ ID NO: 34 - protein, 10 aa, Artificial Sequence
  CD37-12 VL-CDR3
SASSSVTYMH

SEQ ID NO: 35 - protein, 7 aa, Artificial Sequence
  CD37-38 VL-CDR1
DTSKLAS

SEQ ID NO: 36 - protein, 9 aa, Artificial Sequence
  CD37-38 VL-CDR3
QQWISNPPT

SEQ ID NO: 37 - protein, 10 aa, Artificial Sequence
  CD37-50 VL-CDR1
SATSSVTYMH

SEQ ID NO: 38 - protein, 7 aa, Artificial Sequence
  CD37-50 VL-CDR2
DTSKLPY

SEQ ID NO: 39 - protein, 9 aa, Artificial Sequence
  CD37-50 VL-CDR3
QQWSDNPPT

SEQ ID NO: 40 - protein, 7 aa, Artificial Sequence
  Humanized VL-CDR2
DTSNLPY

SEQ ID NO: 41 - protein, 10 aa, Artificial Sequence
  CD37-51 VL-CDR1
SATSSVTYMH

SEQ ID NO: 42 - protein, 7 aa, Artificial Sequence
  CD37-51 VL-CDR2
DTSKLAS

SEQ ID NO: 43 - protein, 9 aa, Artificial Sequence
  CD37-51 VL-CDR3
QQWSSNPPT

SEQ ID NO: 44 - protein, 10 aa, Artificial Sequence
  CD37-56 VL-CDR1
SASSSVTYMH

SEQ ID NO: 45 - protein, 7 aa, Artificial Sequence
  CD37-56 VL-CDR2
DTSKLAS

SEQ ID NO: 46 - protein, 9 aa, Artificial Sequence
  CD37-56 VL-CDR3
QQWISDPPT

SEQ ID NO: 47 - protein, 7 aa, Artificial Sequence
  Humanized VL-CDR2
DTSNLAS

SEQ ID NO: 48 - protein, 10 aa, Artificial Sequence
  CD37-57 VL-CDR1
SATSSVTYMH

SEQ ID NO: 49 - protein, 7 aa, Artificial Sequence
  CD37-57 VL-CDR2
DTSKLAS

SEQ ID NO: 50 - protein, 9 aa, Artificial Sequence
  CD37-57 VL-CDR3
QQWSDNPPT

SEQ ID NO: 51 - protein, 7 aa, Artificial Sequence
  Humanized VL-CDR2
DTSNLAS

SEQ ID NO: 52 - protein, 10 aa, Artificial Sequence
  CONSENSUS VL-CDR1
SAXSSVTYMH

SEQ ID NO: 53 - protein, 7 aa, Artificial Sequence
  CONSENSUS VL-CDR2
DTSXLXX

SEQ ID NO: 54 - protein, 9 aa, Artificial Sequence
  CONSENSUS VL-CDR3
QQWXXXPPT

SEQ ID NO: 55 - protein, 115 aa, Artificial Sequence
  muCD37-3
QVQVKESGPGLVAPSQSLSITCTVSGFSLTTSGVSWVRQPPGKGLEWLGVIWGDGSTNYHSALKSRLSIKKDHSKSQVFLKLNSLQTDDTATYYCAKGGYSLAHWGQGTLVTVSA

SEQ ID NO: 56 - protein, 115 aa, Artificial Sequence; identical to SEQ ID NO: 55
  chCD37-3

SEQ ID NO: 57 - protein, 115 aa, Artificial Sequence
  huCD37-3v1.0
QVQVQESGPGLVAPSQTLSITCTVSGFSLTTSGVSWVRQPPGKGLEWLGVIWGDGSTNYHPSLKSRLSIKKDHSKSQVFLKLNSLTAADTATYYCAKGGYSLAHWGQGTLVTVSS

SEQ ID NO: 58 - protein, 115 aa, Artificial Sequence
  huCD37-3v1.1
QVQVQESGPGLVAPSQTLSITCTVSGFSLTTSGVSWVRQPPGKGLEWLGVIWGDGSTNYHSSLKSRLSIKKDHSKSQVFLKLNSLTAADTATYYCAKGGYSLAHWGQGTLVTVSS

SEQ ID NO: 59 - protein, 115 aa, Artificial Sequence
  muCD37-12
QIQLVQSGPELKKPGETVKISCKASGYTFTKYGMNWVKQAQGKGLKWMGWINTNTGESRNAEEFKGRFAFSLETSASTAYLQINNLKYEDTATYFCGRGTVVADWGQGTTLTVSS

SEQ ID NO: 60 - protein, 115 aa, Artificial Sequence; identical to SEQ ID NO: 59
  chCD37-12

SEQ ID NO: 61 - protein, 120 aa, Artificial Sequence
  muCD37-38
DVQLQESGPDLVKPSQSLSLTCTVTGYSITSGFGWHWIRQFPGNKLEWMAYILYSGGTDYNPSLKSRISITRDTSKNQFFLRLSSVTTEDTATYYCARGYYGYGAWFVYWGQGTLVTVSA

SEQ ID NO: 62 - protein, 120 aa, Artificial Sequence
  chCD37-38
QVQLQESGPDLVKPSQSLSLTCTVTGYSITSGFGWHWIRQFPGNKLEWMAYILYSGGTDYNPSLKSRISITRDTSKNQFFLRLSSVTTEDTATYYCARGYYGYGAWFVYWGQGTLVTVSA

SEQ ID NO: 63 - protein, 120 aa, Artificial Sequence
  huCD37-38
QVQLQESGPGLVKPSQSLSLTCTVSGYSITSGFGWHWIRQFPGKGLEWMAYILYSGGTDYNPSLKSRISITRDTSKNQFFLRLSSVTAADTATYYCARGYYGYGAWFVYWGQGTLVTVSS

SEQ ID NO: 64 - protein, 120 aa, Artificial Sequence
  muCD37-50
DVQLQESGPDLLKPSQSLSLTCTVTGYSITSGFAWHWIRQFPGNKLEWMGYILYSGSTVYSPSLKSRISITRDTSKNHFFLQLNSVTTEDTATYYCARGYYGYGAWFAYWGQGTLVTVSA

SEQ ID NO: 65 - protein, 120 aa, Artificial Sequence
  huCD37-50
QVQLQESGPGLLKPSQSLSLTCTVSGYSITSGFAWHWIRQHPGNKLEWMGYILYSGSTVYSPSLKSRISITRDTSKNHFFLQLNSVTAADTATYYCARGYYGYGAWFAYWGQGTLVTVSA

SEQ ID NO: 66 - protein, 120 aa, Artificial Sequence
  muCD37-51
DVQLQESGPDLLKPSQSLSLTCTVTGYSISSGFAWHWIRQFPGNKLEWMGYIHYSGSTNYSPSLKSRISITRDSSKNQFFLQLNSVTTEDTATYYCARGYYGFGAWFVYWGQGTLVTVSA

SEQ ID NO: 67 - protein, 120 aa, Artificial Sequence
  huCD37-51
EVQLVESGPEVLKPGESLSLTCTVSGYSISSGFAWHWIRQFPGKGLEWMGYIHYSGSTNYSPSLQGRISITRDSSINQFFLQLNSVTASDTATYYCARGYYGFGAWFVYWGQGTLVTVSA

SEQ ID NO: 68 - protein, 120 aa, Artificial Sequence
  muCD37-56
DVQLQESGPDLVKPSQSLSLTCTVTGYSITSGFAWHWIRQFPGNKLEWMGYIHYSGGTNYNPSLKSRVSITRDTSKNQFFLQLNSVTTEDTATYYCARGYYGFGAWFAYWGQGTLVPVSA

SEQ ID NO: 69 - protein, 120 aa, Artificial Sequence
  huCD37-56
QVQLQESGPGLVKPSQSLSLTCTVSGYSITSGFAWHWIRQFPGKGLEWMGYIHYSGGTNYNPSLKSRVSITRDTSKNQFFLQLNSVTAADTATYYCARGYYGFGAWFAYWGQGTLVPVSA

SEQ ID NO: 70 - protein, 120 aa, Artificial Sequence
  muCD37-57
DVQLQESGPDLLKPSQSLSLTCTVTGYSITSGFAWHWIRQFPGNKLEWMGYILYSGSTVYSPSLKSRISITRDTSKNQFFLQLNSVTTEDTATYYCARGYYGYGAWFAYWGQGTLVTVSA

SEQ ID NO: 71 - protein, 120 aa, Artificial Sequence
  huCD37-57
QVQLQESGPGLLKPSQSLSLTCTVSGYSITSGFAWHWIRQFPGKGLEWMGYILYSGSTVYSPSLKSRISITRDTSKNQFFLQLNSVTAADTATYYCARGYYGYGAWFAYWGQGTLVTVSA

SEQ ID NO: 72 - protein, 108 aa, Artificial Sequence
  muCD37-3
DIQMTQSPASLSVSVGETVTITCRASENIRSNLAWYQQKQGKSPQLLVNVATNLADGVPSRFSGSGSGTQYSLKINSLQSEDFGTYYCQHYWGTTWTFGGGTKLEIKR

SEQ ID NO: 73 - protein, 108 aa, Artificial Sequence; identical to SEQ ID NO: 72
  chCD37-3

SEQ ID NO: 74 - protein, 108 aa, Artificial Sequence
  huCD37-3 (1.0 and 1.1)
DIQMTQSPSSLSVSVGERVTITCRASENIRSNLAWYQQKPGKSPKLLVNVATNLADGVPSRFSGSGSGTDYSLKINSLQPEDFGTYYCQHYWGTTWTFGQGTKLEIKR

SEQ ID NO: 75 - protein, 112 aa, Artificial Sequence
  muCD37-12
DIVLTQSPASLAVSLGQRATISCRASQSVSTSSYSYLYWFQQKPGQPPKLLIKYASNLASGVPARFSGSGSGTDFTLNIHPVEEEDTATYYCQHSWEIPYTFGGGTKLEIKR

SEQ ID NO: 76 - protein, 112 aa, Artificial Sequence; identical to SEQ ID NO: 75
  chCD37-12

SEQ ID NO: 77 - protein, 107 aa, Artificial Sequence
  muCD37-38
QIVLTQSPAIMSASPGEKVTMTCSASSSVTYMHWYQQKSGTSPKRWIYDTSKLASGVPARFSGGGSGTSYSLTISSMEAEDAATYYCQQWISNPPTFGGGTKLEIKR

SEQ ID NO: 78 - protein, 107 aa, Artificial Sequence; identical to SEQ ID NO: 77
  chCD37-38

SEQ ID NO: 79 - protein, 107 aa, Artificial Sequence
  huCD37-38
DIVLTQSPASMSASPGERVTMTCSASSSVTYMHWYQQKPGTSPKRWIYDTSKLASGVPARFSGSGSGTSYSLTISSMEAEDAATYYCQQWISNPPTFGGGTKLEIKR

SEQ ID NO: 80 - protein, 106 aa, Artificial Sequence
  muCD37-50
QIVLTQSPAIMSASPGEKVTMTCSATSSVTYMHWYQQKSGTSPKRWIYDTSKLPYGVPGRFSGSGSGTSYSLTISSMEAEDAATYYCQQWSDNPPTFGSGTKLEIK

SEQ ID NO: 81 - protein, 107 aa, Artificial Sequence
  huCD37-50
EIVLTQSPATMSASPGERVTMTCSATSSVTYMHWYQQKPGQSPKRWIYDTSNLPYGVPARFSGSGSGTSYSLTISSMEAEDAATYYCQQWSDNPPTFGQGTKLEIKR

SEQ ID NO: 82 - protein, 107 aa, Artificial Sequence
  muCD37-51
QIVLTQSPAIMSASPGEKVTMTCSATSSVTYMHWYQQKSGTSPKRWIYDTSKLASGVPARFSGSGSGTSYSLTISNMEAEDAATYYCQQWSSNPPTFGSGTKLEIKR

SEQ ID NO: 83 - protein, 107 aa, Artificial Sequence
  huCD37-51
EIVLTQSPATMSASPGERVTMTCSATSSVTYMHWYQQKPGQSPKRWIYDTSKLASGVPARFSGSGSGTSYSLTISSMEAEDAATYYCQQWSSNPPTFGQGTKLEIKR

SEQ ID NO: 84 - protein, 107 aa, Artificial Sequence
  muCD37-56
QIVLTQSPAFMSASPGDKVTMTCSASSSVTYMHWYQQKSGTSPKRWIYDTSKLASGVPARFSGGGSGTSYSLTISTMEAEDAATYYCQQWISDPPTFGGGTKLEIKR

SEQ ID NO: 85 - protein, 107 aa, Artificial Sequence
  huCD37-56
DIVLTQSPAFMSASPGEKVTMTCSASSSVTYMHWYQQKPDQSPKRWIYDTSNLASGVPSRFSGGGSGTDYSLTISSMEAEDAATYYCQQWISDPPTFGQGTKLEIKR

SEQ ID NO: 86 - protein, 107 aa, Artificial Sequence
  muCD37-57
QIVLTQSPAIMSASPGEKVTMTCSATSSVTYMHWYQQKSGTSPKRWIYDTSKLASGVPARFSGSGSGTSYSLTISSMEAEDAATYYCQQWSDNPPTFGSGTKLEIKR

SEQ ID NO: 87 - protein, 107 aa, Artificial Sequence
  huCD37-57
EIVLTQSPATMSASPGERVTMTCSATSSVTYMHWYQQKPGQSPRRWIYDTSNLASGVPARFSGSGSGTSYSLTISSMEAEDAATYYCQQWSDNPPTFGQGTKLEIKR

SEQ ID NO: 88 - protein, 445 aa, Artificial Sequence
  muCD37-3
QVQVKESGPGLVAPSQSLSITCTVSGFSLTTSGVSWVRQPPGKGLEWLGVIWGDGSTNYHSALKSRLSIKKDHSKSQVFLKLNSLQTDDTATYYCAKGGYSLAHWGQGTLVTVSAAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 89 - protein, 445 aa, Artificial Sequence
  chCD37-3
QVQVKESGPGLVAPSQSLSITCTVSGFSLTTSGVSWVRQPPGKGLEWLGVIWGDGSTNYHSALKSRLSIKKDHSKSQVFLKLNSLQTDDTATYYCAKGGYSLAHWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 444 aa, Artificial Sequence
  huCD37-3v1.0
QVQVQESGPGLVAPSQTLSITCTVSGFSLTTSGVSWVRQPPGKGLEWLGVIWGDGSTNYHPSLKSRLSIKKDHSKSQVFLKLNSLTAADTATYYCAKGGYSLAHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 91 - protein, 444 aa, Artificial Sequence
  huCD37-3v1.1
QVQVQESGPGLVAPSQTLSITCTVSGFSLTTSGVSWVRQPPGKGLEWLGVIWGDGSTNYHSSLKSRLSIKKDHSKSQVFLKLNSLTAADTATYYCAKGGYSLAHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 92 - protein, 445 aa, Artificial Sequence
  muCD37-12
QIQLVQSGPELKKPGETVKISCKASGYTFTKYGMNWVKQAQGKGLKWMGWINTNTGESRNAEEFKGRFAFSLETSASTAYLQINNLKYEDTATYFCGRGTVVADWGQGTTLTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 93 - protein, 445 aa, Artificial Sequence
  chCD37-12
QIQLVQSGPELKKPGETVKISCKASGYTFTKYGMNWVKQAQGKGLKWMGWINTNTGESRNAEEFKGRFAFSLETSASTAYLQINNLKYEDTATYFCGRGTVVADWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 444 aa, Artificial Sequence
  muCD37-38
DVQLQESGPDLVKPSQSLSLTCTVTGYSITSGFGWHWIRQFPGNKLEWMAYILYSGGTDYNPSLKSRISITRDTSKNQFFLRLSSVTTEDTATYYCARGYYGYGAWFVYWGQGTLVTVSAAKTTPPSVYPLAPGSAAQTNSMVTLGCLVKGYFPEPVTVTWNSGSLSSGVHTFPAVLESDLYTLSSSVTVPSSMRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 95 - protein, 449 aa, Artificial Sequence
  chCD37-38
QVQLQESGPDLVKPSQSLSLTCTVTGYSITSGFGWHWIRQFPGNKLEWMAYILYSGGTDYNPSLKSRISITRDTSKNQFFLRLSSVTTEDTATYYCARGYYGYGAWFVYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 96 - protein, 449 aa, Artificial Sequence
  huCD37-38
QVQLQESGPGLVKPSQSLSLTCTVSGYSITSGFGWHWIRQFPGKGLEWMAYILYSGGTDYNPSLKSRISITRDTSKNQFFLRLSSVTAADTATYYCARGYYGYGAWFVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 97 - protein, 450 aa, Artificial Sequence
  muCD37-50
DVQLQESGPDLLKPSQSLSLTCTVTGYSITSGFAWHWIRQFPGNKLEWMGYILYSGSTVYSPSLKSRISITRDTSKNHFFLQLNSVTTEDTATYYCARGYYGYGAWFAYWGQGTLVTVSAAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 98 - protein, 449 aa, Artificial Sequence
  huCD37-50
QVQLQESGPGLLKPSQSLSLTCTVSGYSITSGFAWHWIRQHPGNKLEWMGYILYSGSTVYSPSLKSRISITRDTSKNHFFLQLNSVTAADTATYYCARGYYGYGAWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 99 - protein, 450 aa, Artificial Sequence
  muCD37-51
DVQLQESGPDLLKPSQSLSLTCTVTGYSISSGFAWHWIRQFPGNKLEWMGYIHYSGSTNYSPSLKSRISITRDSSKNQFFLQLNSVTTEDTATYYCARGYYGFGAWFVYWGQGTLVTVSAAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 100 - protein, 449 aa, Artificial Sequence
  huCD37-51
EVQLVESGPEVLKPGESLSLTCTVSGYSISSGFAWHWIRQFPGKGLEWMGYIHYSGSTNYSPSLQGRISITRDSSINQFFLQLNSVTASDTATYYCARGYYGFGAWFVYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 101 - protein, 444 aa, Artificial Sequence
  muCD37-56
DVQLQESGPDLVKPSQSLSLTCTVTGYSITSGFAWHWIRQFPGNKLEWMGYIHYSGGTNYNPSLKSRVSITRDTSKNQFFLQLNSVTTEDTATYYCARGYYGFGAWFAYWGQGTLVPVSAAKTTPPSVYPLAPGSAAQTNSMVTLGCLVKGYFPEPVTVTWNSGSLSSGVHTFPAVLESDLYTLSSSVTVPSSMRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 102 - protein, 449 aa, Artificial Sequence
  huCD37-56
QVQLQESGPGLVKPSQSLSLTCTVSGYSITSGFAWHWIRQFPGKGLEWMGYIHYSGGTNYNPSLKSRVSITRDTSKNQFFLQLNSVTAADTATYYCARGYYGFGAWFAYWGQGTLVPVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 103 - protein, 450 aa, Artificial Sequence
  muCD37-57
DVQLQESGPDLLKPSQSLSLTCTVTGYSITSGFAWHWIRQFPGNKLEWMGYILYSGSTVYSPSLKSRISITRDTSKNQFFLQLNSVTTEDTATYYCARGYYGYGAWFAYWGQGTLVTVSAAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 104 - protein, 449 aa, Artificial Sequence
  huCD37-57
QVQLQESGPGLLKPSQSLSLTCTVSGYSITSGFAWHWIRQFPGKGLEWMGYILYSGSTVYSPSLKSRISITRDTSKNQFFLQLNSVTAADTATYYCARGYYGYGAWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 105 - protein, 214 aa, Artificial Sequence
  muCD37-3
DIQMTQSPASLSVSVGETVTITCRASENIRSNLAWYQQKQGKSPQLLVNVATNLADGVPSRFSGSGSGTQYSLKINSLQSEDFGTYYCQHYWGTTWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 106 - protein, 214 aa, Artificial Sequence
  chCD37-3
DIQMTQSPASLSVSVGETVTITCRASENIRSNLAWYQQKQGKSPQLLVNVATNLADGVPSRFSGSGSGTQYSLKINSLQSEDFGTYYCQHYWGTTWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 107 - protein, 214 aa, Artificial Sequence
  huCD37-3 (1.0 and 1.1)
DIQMTQSPSSLSVSVGERVTITCRASENIRSNLAWYQQKPGKSPKLLVNVATNLADGVPSRFSGSGSGTDYSLKINSLQPEDFGTYYCQHYWGTTWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 108 - protein, 218 aa, Artificial Sequence
  muCD37-12
DIVLTQSPASLAVSLGQRATISCRASQSVSTSSYSYLYWFQQKPGQPPKLLIKYASNLASGVPARFSGSGSGTDFTLNIHPVEEEDTATYYCQHSWEIPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 109 - protein, 218 aa, Artificial Sequence
  chCD37-12
DIVLTQSPASLAVSLGQRATISCRASQSVSTSSYSYLYWFQQKPGQPPKLLIKYASNLASGVPARFSGSGSGTDFTLNIHPVEEEDTATYYCQHSWEIPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 110 - protein, 213 aa, Artificial Sequence
  muCD37-38
QIVLTQSPAIMSASPGEKVTMTCSASSSVTYMHWYQQKSGTSPKRWIYDTSKLASGVPARFSGGGSGTSYSLTISSMEAEDAATYYCQQWISNPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 111 - protein, 213 aa, Artificial Sequence
  chCD37-38
QIVLTQSPAIMSASPGEKVTMTCSASSSVTYMHWYQQKSGTSPKRWIYDTSKLASGVPARFSGGGSGTSYSLTISSMEAEDAATYYCQQWISNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 112 - protein, 213 aa, Artificial Sequence
  huCD37-38
DIVLTQSPASMSASPGERVTMTCSASSSVTYMHWYQQKPGTSPKRWIYDTSKLASGVPARFSGSGSGTSYSLTISSMEAEDAATYYCQQWISNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 113 - protein, 213 aa, Artificial Sequence
  muCD37-50
QIVLTQSPAIMSASPGEKVTMTCSATSSVTYMHWYQQKSGTSPKRWIYDTSKLPYGVPGRFSGSGSGTSYSLTISSMEAEDAATYYCQQWSDNPPTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 114 - protein, 213 aa, Artificial Sequence
  huCD37-50
EIVLTQSPATMSASPGERVTMTCSATSSVTYMHWYQQKPGQSPKRWIYDTSNLPYGVPARFSGSGSGTSYSLTISSMEAEDAATYYCQQWSDNPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - protein, 213 aa, Artificial Sequence
  muCD37-51
QIVLTQSPAIMSASPGEKVTMTCSATSSVTYMHWYQQKSGTSPKRWIYDTSKLASGVPARFSGSGSGTSYSLTISNMEAEDAATYYCQQWSSNPPTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 116 - protein, 213 aa, Artificial Sequence
  huCD37-51
EIVLTQSPATMSASPGERVTMTCSATSSVTYMHWYQQKPGQSPKRWIYDTSKLASGVPARFSGSGSGTSYSLTISSMEAEDAATYYCQQWSSNPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 117 - protein, 213 aa, Artificial Sequence
  muCD37-56
QIVLTQSPAFMSASPGDKVTMTCSASSSVTYMHWYQQKSGTSPKRWIYDTSKLASGVPARFSGGGSGTSYSLTISTMEAEDAATYYCQQWISDPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 118 - protein, 213 aa, Artificial Sequence
  huCD37-56
DIVLTQSPAFMSASPGEKVTMTCSASSSVTYMHWYQQKPDQSPKRWIYDTSNLASGVPSRFSGGGSGTDYSLTISSMEAEDAATYYCQQWISDPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 119 - protein, 213 aa, Artificial Sequence
  muCD37-57
QIVLTQSPAIMSASPGEKVTMTCSATSSVTYMHWYQQKSGTSPKRWIYDTSKLASGVPARFSGSGSGTSYSLTISSMEAEDAATYYCQQWSDNPPTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 120 - protein, 212 aa, Artificial Sequence
  huCD37-57
EIVLTQSPATMSASPGERVTMTCSATSSVTYMHWYQQKPGQSPRRWIYDTSNLASGVPARFSGSGSGTSYSLTISSMEAEDAATYYCQQWSDNPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 121 - DNA, 345 bp, Artificial Sequence
  muCD37-3
caggtgcaggtgaaggagtcaggacctggcctggtggcgccctcacagagcctgtccattacatgcactgtctcagggttctcattaaccacctctggtgtaagctgggttcgccagcctccaggaaagggtctggagtggctgggagtaatatggggtgacgggagcacaaactatcattcagctctcaaatccagactgagcatcaagaaggatcactccaagagccaagttttcttaaaactgaacagtctgcaaactgatgacacagccacgtactactgtgccaaaggaggctactcgttggctcactggggccaagggactctggtcacagtctctgca

SEQ ID NO: 122 - DNA, 431 bp, Artificial Sequence
  chCD37-3
aagcttgccaccatggctgtcctggcactgctcctctgcctggtgacatacccaagctgtgtcctatcacaggtgcaggtgaaggagtcaggacctggcctggtggcgccctcacagagcctgtccattacatgcactgtctcagggttctcattaaccacctctggtgtaagctgggttcgccagcctccaggaaagggtctggagtggctgggagtaatatggggtgacgggagcacaaactatcattcagctctcaaatccagactgagcatcaagaaggatcactccaagagccaagttttcttaaaactgaacagtctgcaaactgatgacacagccacgtactactgtgccaaaggaggctactcgttggctcactggggccaagggactctggtcacagtctctgcagcctctacgaagggccc

SEQ ID NO: 123 - DNA, 431 bp, Artificial Sequence
  huCD37-3v1.0
aagcttgccaccatgggttggagctgcattattctgtttctggtggccaccgccaccggtgtgcactcacaagtccaagtccaagaatctggtccaggtctggtggccccttcccaaactctgagcatcacctgtaccgtttctggttttagccttaccacctctggtgtgagttgggtacgccaaccacccggtaagggtctcgaatggctgggtgtaatctggggtgatggttccacaaattaccatccttccctcaagtcccgccttagcatcaaaaaggatcacagcaaaagtcaagttttcctgaaactgaatagtctgacagcagccgatacagccacctactattgcgccaagggtggttatagtcttgcacactggggtcaaggtaccctcgttaccgtctcctcagctagtaccaagggccc

SEQ ID NO: 124 - DNA, 431 bp, Artificial Sequence
  huCD37-3v1.1
aagcttgccaccatgggctggagctgtatcattctgtttctggtggcgacagctactggggtccactcccaagtgcaggtacaagagtccgggcctggattggtcgcaccaagccagaccctctctatcacttgtaccgttagcgggttctctctgacaaccagtggagtgagttgggtgaggcagccaccaggaaagggactggagtggctgggggtgatttggggcgacggcagcacaaactatcattccagtcttaaatctcggttgtccattaaaaaagaccatagtaaatctcaagttttcctgaaactcaatagcctgacagccgcagacactgctacgtattactgcgccaaaggaggatacagtctggctcactggggacaggggaccctggtgaccgtgtcatccgcatcaacaaagggccc

SEQ ID NO: 125 - DNA, 345 bp, Artificial Sequence
  muCD37-12
cagatccagttggtgcagtctggacctgagctgaagaagcctggagagacagtcaagatctcctgcaaggcttctgggtataccttcacaaagtatggaatgaactgggtgaagcaggctcaaggaaagggtttaaagtggatgggctggataaacaccaacactggagagtcaagaaatgctgaagaattcaagggacggtttgccttctctttggaaacctctgccagcactgcctatttgcagatcaacaacctcaaatatgaggacacggctacatatttctgtggaaggggcacggtagtagcggactggggccaaggcaccactctcacagtctcctca

SEQ ID NO: 126 - DNA, 431 bp, Artificial Sequence
  chCD37-12
aagcttgccaccatggggtggtcatgcataatcctctttctggtcgctactgctaccggtgtgcactcacagattcagctggttcaaagtggcccagagctgaaaaagccaggggaaacagtgaaaataagttgcaaggcatccggttacactttcacaaagtacggcatgaactgggtcaagcaggcccagggcaaggggctcaaatggatgggttggatcaataccaacactggcgagtctaggaatgctgaggagtttaagggccggtttgccttcagcctggagacaagtgccagcacagcttacctgcaaatcaacaatctgaagtatgaggatacagcaacctatttctgcggccgcggcactgtcgttgcagactggggacaaggtaccaccttgactgtatccagtgccagcactaagggccc

SEQ ID NO: 127 - DNA, 360 bp, Artificial Sequence
  muCD37-38
gatgtgcagcttcaggagtcaggacctgacctggtgaaaccttctcagtcactttcactcacctgcactgtcactggctactccatcaccagtggttttggctggcactggatccggcagtttccaggaaacaagctggaatggatggcctacatactctacagtggtggcactgactacaacccatctctcaaaagtcgaatctctatcactcgagacacttccaagaaccagttcttcctgcggttgagttctgtgactactgaggacacagccacatattactgtgcaagaggctactatggttacggggcctggtttgtttactggggccaagggactctggtcactgtctctgca

SEQ ID NO: 128 - DNA, 446 bp, Artificial Sequence
  chCD37-38
aagcttgccaccatgggctggagttgtatcattctgtttttggtggccaccgccactggagtccattcccaagtgcaactccaggaatctggccctgacctggttaagccatctcagagcctctccctgacctgcactgttacaggatactcaatcacatcaggctttggctggcactggatcagacaatttcccgggaacaagttggaatggatggcttacattctgtatagcgggggtaccgattacaatccttccctcaagagccgaatctctatcaccagggatacaagcaagaaccaattttttctccgcctcagctctgtgactaccgaagataccgctacttactattgtgccaggggctactatggatatggtgcatggttcgtctattggggccagggaaccctggtgactgtgagcgctgcctctaccaagggccc

SEQ ID NO: 129 - DNA, 446 bp, Artificial Sequence
  huCD37-38
aagcttgccaccatgggttggagctgcatcattcttttcctggtcgctactgcaactggagtccactcacaggtccagctgcaagagtccggtcctgggcttgtgaaacccagccagtccctcagtctcacctgtactgtctctggctactctattaccagtgggttcggctggcattggattaggcagtttcccggtaaggggctggagtggatggcatatatcctgtacagcggaggaaccgattacaacccaagtctgaagagcaggatcagcattacccgggacacaagcaaaaaccagtttttccttcggctgtctagtgttacagctgcagacaccgctacttactattgtgctcggggttactatggctatggggcttggtttgtgtattggggacaaggcactcttgtgaccgtgagcagcgcctcaacaaagggccc

SEQ ID NO: 130 - DNA, 360 bp, Artificial Sequence
  muCD37-50
gatgtgcagcttcaggagtcaggacctgacctgttgaaaccttctcagtcactttcactcacctgcactgtcactggctactccatcaccagtggttttgcctggcactggatccggcagtttccaggaaacaaactggaatggatgggctacatactctacagtggtagcactgtctacagcccatctctcaaaagtcgaatctctatcactcgagacacatccaagaaccacttcttcctgcagttgaattctgtgactactgaggacacagccacatattactgtgcaagagggtactatggttacggcgcctggtttgcttactggggccaagggactctggtcactgtctctgca

SEQ ID NO: 131 - DNA, 360 bp, Artificial Sequence
  muCD37-51
gatgtgcagcttcaggagtcaggacctgacctgttgaaaccttctcagtcactttcactcacctgcactgtcactggctactccatctccagtggttttgcctggcactggatccggcagtttccaggaaacaaactggaatggatgggctacatacactacagtggtagcactaactacagcccatctctcaaaagtcgaatctctatcactcgagactcatccaagaaccagttcttcctgcagttgaattctgtgactactgaggacacagccacatattactgtgcaagaggatactatggtttcggcgcctggtttgtttactggggccaagggactctggtcactgtctctgca

SEQ ID NO: 132 - DNA, 360 bp, Artificial Sequence
  muCD37-56
gatgtgcagcttcaggagtcaggacctgacctggtgaaaccttctcagtcactttcactcacctgcactgtcactggctactccatcaccagtggttttgcctggcactggatccggcagtttccaggaaacaaactggaatggatgggctacatacactacagtggtggcactaactacaacccatctctcaaaagtcgagtctctatcactcgagacacatccaagaaccagttcttcctgcagttgaattctgtgactactgaggacacagccacatattactgtgcaagaggctactatggtttcggggcctggtttgcttactggggccaagggactctggtccctgtctctgca

SEQ ID NO: 133 - DNA, 446 bp, Artificial Sequence
  huCD37-56
aagcttgccaccatggggtggagctgcattatcctgttcctcgtcgccaccgcaaccggcgtccactcccaggtgcagctgcaagaaagcgggccaggattggtaaaaccttcccagtctctgagtcttacttgtaccgtatctggatacagtatcacatctggcttcgcctggcattggattcgccagtttcccggcaaggggcttgagtggatggggtatattcattattctggaggtaccaactacaacccttccctgaagagtcgagtctcaattaccagggacacttccaagaaccaattctttttgcagcttaattcagtgaccgctgccgacaccgctacttactactgcgcccggggctactatgggtttggtgcctggttcgcctactggggccaggggaccctggtgcccgtgtctgctgcctccacaaagggccc

SEQ ID NO: 134 - DNA, 360 bp, Artificial Sequence
  muCD37-57
gatgtgcagcttcaggagtcaggacctgacctgttgaaaccttctcagtcactttcactcacctgcactgtcactggctactccatcaccagtggttttgcctggcactggatccggcagtttccaggaaacaaactggaatggatgggctacatactctacagtggtagcactgtctacagcccatctctcaaaagtcgaatctctatcactcgagacacatccaagaaccagttcttcctgcagttgaattctgtgactactgaggacacagccacatattactgtgcaagagggtactatggttacggcgcctggtttgcttactggggccaagggactctggtcactgtctctgca

SEQ ID NO: 135 - DNA, 446 bp, Artificial Sequence
  huCD37-57
aagcttgccaccatgggctggagctgcatcattctgtttctggtggccacagcaactggcgttcacagtcaagtccaactgcaggagagcggccccggactcctgaaaccatctcagtcactcagtctgacatgtactgtgagcggctacagcattacctcaggcttcgcttggcattggatcaggcagttccccggaaaaggtctggagtggatggggtacattctgtacagcggcagtacagtgtattcaccctccttgaaatctaggatatcaatcacacgtgatacaagcaaaaatcagttcttcctccagctgaactccgtcaccgccgcagacacagcaacctattattgtgctcgcggatactacggatatggcgcatggttcgcctattggggccaggggacactcgtgaccgtttccgccgcctccacaaagggccc

SEQ ID NO: 136 - DNA, 324 bp, Artificial Sequence
  muCD37-3
gacatccagatgactcagtctccagcctccctttctgtatctgtgggagaaactgtcaccatcacatgtcgagcaagtgagaatattcgcagtaatttagcatggtatcagcagaaacagggaaaatctcctcagctcctggtcaatgttgcaacaaacttagcagatggtgtgccatcaaggttcagtggcagtggatcaggcacacagtattccctcaagatcaacagcctgcagtctgaagattttgggacttattactgtcaacattattggggtactacgtggacgttcggtggaggcaccaagctggaaatcaaacgt

SEQ ID NO: 137 - DNA, 399 bp, Artificial Sequence
  chCD37-3
gaattcgccaccatgagtgtgcccactcaggtcctggggttgctgctgctgtggcttacagatgccagatgtgacatccagatgactcagtctccagcctccctttctgtatctgtgggagaaactgtcaccatcacatgtcgagcaagtgagaatattcgcagtaatttagcatggtatcagcagaaacagggaaaatctcctcagctcctggtcaatgttgcaacaaacttagcagatggtgtgccatcaaggttcagtggcagtggatcaggcacacagtattccctcaagatcaacagcctgcagtctgaagattttgggacttattactgtcaacattattggggtactacgtggacgttcggtggaggcaccaagctggaaatcaaacgtacg

SEQ ID NO: 138 - DNA, 396 bp, Artificial Sequence
  huCD37-3 (1.0 and 1.1)
gaattcgccaccatgggttggtcctgcatcatcttgtttctcgtggccacagccaccggtgttcactctgatatacaaatgactcaaagcccttccagtttgagcgtaagtgtgggtgaacgcgtaacaatcacctgtagagctagtgaaaacatccgcagtaatctcgcatggtaccaacaaaagccaggtaagtcacctaagctcctcgtgaatgttgctaccaacctcgctgatggtgtgccttcacgattctctggttcaggttccggtaccgattattcacttaagatcaactcactccaaccagaagatttcggtacatattactgtcaacactactggggtacgacctggacattcggtcaaggtactaagctggaaatcaagcgtacg

SEQ ID NO: 139 - DNA, 336 bp, Artificial Sequence
  muCD37-12
gacattgtgctaacacagtctcctgcttccttagctgtatctctggggcagagggccaccatctcatgcagggccagccaaagtgtcagtacatctagctatagttatttgtactggttccagcagaaaccaggacagccacccaaactcctcatcaagtatgcatccaacctagcatctggggtccctgccaggttcagtggcagtgggtctgggacagacttcaccctcaacatccatcctgtggaggaggaggatactgcaacatattactgtcaacacagttgggagattccgtacacgttcggaggggggaccaaactggaaataaaacgg

SEQ ID NO: 140 - DNA, 408 bp, Artificial Sequence
  chCD37-12
gaattcgccaccatgggttggtcctgtataatcctgttcttggtggccaccgctactggcgttcatagtgatattgtactcactcagtcaccagccagtctggcagtgtccctgggccagcgtgccaccatctcctgccgggcctcacagtccgtgagcactagctcttattcctatctctactggtttcaacagaagccaggacagccccctaagctgctgatcaagtacgcctccaacctcgccagcggcgttcccgctagattctctggttccggtagcggaactgatttcactttgaacatccaccccgttgaggaagaggataccgccacttactattgtcaacactcttgggagattccttacacctttggaggaggaacaaagctcgaaattaagcgtacg

SEQ ID NO: 141 - DNA, 321 bp, Artificial Sequence
  muCD37-38
caaattgttctcacccagtctccagcaatcatgtctgcatctccaggggagaaggtcaccatgacctgcagtgccagctcaagtgtaacttacatgcactggtaccagcagaagtcaggcacctcccccaaaagatggatttatgacacatccaaactggcttctggagtccctgctcgcttcagtggcggtgggtctgggacctcttactctctcacaatcagcagcatggaggctgaagatgctgccacttattactgccagcagtggattagtaacccacccacgttcggaggggggaccaagctggaaattaaacgg

SEQ ID NO: 142 - DNA, 393 bp, Artificial Sequence
  chCD37-38
gaattcgccaccatgggctggtcctgtatcatcctgtttctcgtggccacagctacaggtgttcattctcagattgtgctgacccaatcaccagctattatgtccgctagccccggcgagaaagtgacaatgacatgtagcgctagctcttctgtgacttacatgcattggtatcaacagaagtcaggtaccagtcccaagcgttggatctacgacacatccaaactggcctccggagtccctgccaggttcagcggaggtgggtccggcaccagttattcactgaccatatcctctatggaagctgaagatgctgctacttattattgtcaacaatggatttctaacccccccacctttggtggcggaacaaagctggagatcaagcgtacg

SEQ ID NO: 143 - DNA, 393 bp, Artificial Sequence
  huCD37-38
gaattcgccaccatgggatggtcctgcattattctgttcttggtcgccactgctactggcgttcactctgacattgtgctcacacagtctccagcctcaatgtctgcttcccccggtgagcgggtgaccatgacatgctctgccagttcctccgtgacatatatgcattggtatcagcaaaaacccggtacctctccaaaaagatggatctacgacacttcaaagcttgcatcaggcgttcctgccagattttccgggtctgggtctggcacttcatacagtctgaccattagttccatggaagctgaagatgcagccacctattactgtcagcagtggatttcaaatcctcctaccttcggcggcggaaccaaactggagataaagcgtacg

SEQ ID NO: 144 - DNA, 321 bp, Artificial Sequence
  muCD37-50
caaattgttctcacccagtctccagcaatcatgtctgcatctccaggggagaaggtcaccatgacctgcagtgccacctcaagtgtgacttacatgcactggtaccagcagaagtcaggcacctcccccaaaagatggatttatgacacatccaaactgccttatggagtccctggtcgtttcagtggtagtgggtctgggacctcttactctctcacaatcagcagcatggaggctgaagatgctgccacttattactgccagcagtggagtgataacccacccacgttcggctcggggacaaagttggaaataaagcgg

SEQ ID NO: 145 - DNA, 393 bp, Artificial Sequence
  huCD37-50
gaattcgccaccatgggttggtcatgcattattctgttcctggttgctaccgcaacaggagtacatagtgagatagtcctcacccaaagtcctgctactatgtctgccagcccaggagagcgtgtgaccatgacttgctctgcaacctcaagtgtgacatacatgcattggtatcagcaaaagcctggccaatcccctaaaaggtggatctacgatacttctaatctgccatacggtgtgcccgcaaggttctccgggagtggcagtggcaccagttatagtctgaccatcagttcaatggaagcagaggatgcagcaacctattattgtcagcagtggtccgataatccccctacttttggtcagggtacaaagctggagattaagcgtacg

SEQ ID NO: 146 - DNA, 321 bp, Artificial Sequence
  muCD37-51
caaattgttctcacccagtctccagcaatcatgtctgcatctccaggggagaaggtcaccatgacctgcagtgccacctcaagtgtgacttacatgcactggtaccagcagaagtcaggcacctcccccaaaagatggatttatgacacatccaaactggcttctggagtccctgctcgcttcagtggcagtgggtctgggacctcttactctctcacaatcagcaacatggaggctgaagatgctgccacttattactgccagcagtggagtagtaacccacccacgttcggctcggggacaaagttggaaataaagcgg

SEQ ID NO: 147 - DNA, 393 bp, Artificial Sequence
  huCD37-51
gaattcgccaccatgggatggagctgtattattctgttcctggttgctactgctactggcgtccattccgagatagtcctcacccagagccccgcaaccatgagtgcctcccctggggagcgagtgactatgacttgttccgccacttcttcagttacctatatgcattggtatcagcagaaacctggacagtctccaaagcgttggatttacgacacctccaacctggcttcaggagttcctgctaggttcagcggatctgggtctggcacaagttattcactcaccattagttccatggaggccgaagatgccgctacttactactgtcagcagtggagcagcaacccccctacattcgggcagggaactaagctggagatcaaacgtacg

SEQ ID NO: 148 - DNA, 321 bp, Artificial Sequence
  muCD37-56
caaattgttctcacccagtctccagcattcatgtctgcatctccaggggataaggtcaccatgacctgcagtgccagttcaagtgttacttacatgcactggtatcagcagaagtcaggcacctcccccaaaagatggatttatgacacatccaaactggcttctggagtccctgctcgcttcagtggcggtgggtctgggacctcttactctctcacaatcagcaccatggaggctgaagatgctgccacttattactgccagcagtggattagtgacccacccacgttcggaggggggaccaagctggaaataaaacgg

SEQ ID NO: 149 - DNA, 393 bp, Artificial Sequence
  huCD37-56
gaattcgccaccatgggctggtcctgtatcatcctgtttctggtggcaaccgctactggggttcactctgatattgtcctgacacagagtccagccttcatgagtgcttctcccggagaaaaggtcacaatgacttgttcagcttcctcctccgtcacatacatgcattggtaccagcagaagcctgaccagagtcctaagaggtggatctatgatacaagcaatctggcttccggtgtcccctcccgcttttcaggcggcggaagcggaactgactatagccttaccatctcctcaatggaagccgaggacgctgctacatattactgccagcaatggatcagcgaccctcctactttcggacagggaacaaaattggaaattaagcgtacg

SEQ ID NO: 150 - DNA, 321 bp, Artificial Sequence
  muCD37-57
caaattgttctcacccagtctccagcaatcatgtctgcatctccaggggagaaggtcaccatgacctgcagtgccacctcaagtgtgacttacatgcactggtaccagcagaagtcaggcacctcccccaaaagatggatttatgacacatccaaactggcttctggagtccctgctcgcttcagtggcagtgggtctgggacctcttactctctcacaatcagcagcatggaggctgaagatgctgccacttattactgccagcagtggagtgataacccacccacgttcggctcggggacaaagttggaaataaagcgg

SEQ ID NO: 151 - DNA, 393 bp, Artificial Sequence
  huCD37-57
gaattcgccaccatggggtggtcctgtattatcctgttcctggtcgcaaccgccacaggcgttcactccgagatcgtgttgactcagagcccagccaccatgtccgcttcccccggggagagagtgacaatgacttgttccgccacaagttctgtaacctacatgcattggtaccagcaaaaaccaggacagagtccccgtcgttggatttatgatacctctaacctggcttcaggcgttcctgcccgcttttctggtagtggatctgggacttcctatagccttaccataagctctatggaagccgaggacgccgctacatactactgccagcagtggagtgataacccccccaccttcgggcagggaaccaaattggagatcaaacgtacg

SEQ ID NO: 152 - DNA, 1410 bp, Artificial Sequence
  chCD37-3
aagcttgccaccatggctgtcctggcactgctcctctgcctggtgacatacccaagctgtgtcctatcacaggtgcaggtgaaggagtcaggacctggcctggtggcgccctcacagagcctgtccattacatgcactgtctcagggttctcattaaccacctctggtgtaagctgggttcgccagcctccaggaaagggtctggagtggctgggagtaatatggggtgacgggagcacaaactatcattcagctctcaaatccagactgagcatcaagaaggatcactccaagagccaagttttcttaaaactgaacagtctgcaaactgatgacacagccacgtactactgtgccaaaggaggctactcgttggctcactggggccaagggactctggtcacagtctctgcagcctctacgaagggcccatcagttttccccttggctccaagttctaaatccacaagcggtggaacagctgcactgggatgcctcgttaaagattatttccctgagcctgtgacagtgagctggaatagcggagcattgacttcaggtgtgcacacttttcccgctgtgttgcagtcctccggtctgtactcactgtccagtgtcgtaaccgtcccttctagcagcttgggaacccagacctacatctgtaacgtcaaccataaaccatccaacacaaaggtggataagaaggttgaaccaaagagctgtgataagacacatacatgccctccttgtcctgcaccagagctcctcggaggtccatctgtgttcctgtttccccccaaacccaaggacactcttatgatctctcgtactccagaggtcacctgtgttgttgtcgacgtgagccatgaagatcccgaggttaaattcaactggtacgtggatggagtcgaggttcacaatgccaagaccaagcccagggaggagcaatataattctacatatcgggtagtgagcgttctgaccgtgctccaccaagattggctcaatggaaaagagtacaagtgcaaggtgtccaacaaggctcttcccgctcccattgagaaaactatctccaaagccaaggggcagccacgggaaccccaggtgtatacattgcccccatctagagacgagctgaccaagaaccaggtgagtctcacttgtctggtcaaggggttttacccttctgacattgctgtagagtgggagtctaacggacagccagaaaacaactacaagacaactcccccagtgctggacagcgacgggagcttcttcctctactccaagttgactgtagacaagtctagatggcagcaaggaaacgttttctcctgctcagtaatgcatgaggctctgcacaatcactatacccagaaatcactgtcccttagcccagggtgactcgag

SEQ ID NO: 153 - DNA, 1410 bp, Artificial Sequence
  huCD37-3v1.0
aagcttgccaccatgggttggagctgcattattctgtttctggtggccaccgccaccggtgtgcactcacaagtccaagtccaagaatctggtccaggtctggtggccccttcccaaactctgagcatcacctgtaccgtttctggttttagccttaccacctctggtgtgagttgggtacgccaaccacccggtaagggtctcgaatggctgggtgtaatctggggtgatggttccacaaattaccatccttccctcaagtcccgccttagcatcaaaaaggatcacagcaaaagtcaagttttcctgaaactgaatagtctgacagcagccgatacagccacctactattgcgccaagggtggttatagtcttgcacactggggtcaaggtaccctcgttaccgtctcctcagctagtaccaagggcccatcagttttccccttggctccaagttctaaatccacaagcggtggaacagctgcactgggatgcctcgttaaagattatttccctgagcctgtgacagtgagctggaatagcggagcattgacttcaggtgtgcacacttttcccgctgtgttgcagtcctccggtctgtactcactgtccagtgtcgtaaccgtcccttctagcagcttgggaacccagacctacatctgtaacgtcaaccataaaccatccaacacaaaggtggataagaaggttgaaccaaagagctgtgataagacacatacatgccctccttgtcctgcaccagagctcctcggaggtccatctgtgttcctgtttccccccaaacccaaggacactcttatgatctctcgtactccagaggtcacctgtgttgttgtcgacgtgagccatgaagatcccgaggttaaattcaactggtacgtggatggagtcgaggttcacaatgccaagaccaagcccagggaggagcaatataattctacatatcgggtagtgagcgttctgaccgtgctccaccaagattggctcaatggaaaagagtacaagtgcaaggtgtccaacaaggctcttcccgctcccattgagaaaactatctccaaagccaaggggcagccacgggaaccccaggtgtatacattgcccccatctagagacgagctgaccaagaaccaggtgagtctcacttgtctggtcaaggggttttacccttctgacattgctgtagagtgggagtctaacggacagccagaaaacaactacaagacaactcccccagtgctggacagcgacgggagcttcttcctctactccaagttgactgtagacaagtctagatggcagcaaggaaacgttttctcctgctcagtaatgcatgaggctctgcacaatcactatacccagaaatcactgtcccttagcccagggtgactcgag

SEQ ID NO: 154 - DNA, 1410 bp, Artificial Sequence
  huCD37-3v1.1
aagcttgccaccatgggctggagctgtatcattctgtttctggtggcgacagctactggggtccactcccaagtgcaggtacaagagtccgggcctggattggtcgcaccaagccagaccctctctatcacttgtaccgttagcgggttctctctgacaaccagtggagtgagttgggtgaggcagccaccaggaaagggactggagtggctgggggtgatttggggcgacggcagcacaaactatcattccagtcttaaatctcggttgtccattaaaaaagaccatagtaaatctcaagttttcctgaaactcaatagcctgacagccgcagacactgctacgtattactgcgccaaaggaggatacagtctggctcactggggacaggggaccctggtgaccgtgtcatccgcatcaacaaagggcccatcagttttccccttggctccaagttctaaatccacaagcggtggaacagctgcactgggatgcctcgttaaagattatttccctgagcctgtgacagtgagctggaatagcggagcattgacttcaggtgtgcacacttttcccgctgtgttgcagtcctccggtctgtactcactgtccagtgtcgtaaccgtcccttctagcagcttgggaacccagacctacatctgtaacgtcaaccataaaccatccaacacaaaggtggataagaaggttgaaccaaagagctgtgataagacacatacatgccctccttgtcctgcaccagagctcctcggaggtccatctgtgttcctgtttccccccaaacccaaggacactcttatgatctctcgtactccagaggtcacctgtgttgttgtcgacgtgagccatgaagatcccgaggttaaattcaactggtacgtggatggagtcgaggttcacaatgccaagaccaagcccagggaggagcaatataattctacatatcgggtagtgagcgttctgaccgtgctccaccaagattggctcaatggaaaagagtacaagtgcaaggtgtccaacaaggctcttcccgctcccattgagaaaactatctccaaagccaaggggcagccacgggaaccccaggtgtatacattgcccccatctagagacgagctgaccaagaaccaggtgagtctcacttgtctggtcaaggggttttacccttctgacattgctgtagagtgggagtctaacggacagccagaaaacaactacaagacaactcccccagtgctggacagcgacgggagcttcttcctctactccaagttgactgtagacaagtctagatggcagcaaggaaacgttttctcctgctcagtaatgcatgaggctctgcacaatcactatacccagaaatcactgtcccttagcccagggtgactcgag

SEQ ID NO: 155 - DNA, 1410 bp, Artificial Sequence
  chCD37-12
aagcttgccaccatggggtggtcatgcataatcctctttctggtcgctactgctaccggtgtgcactcacagattcagctggttcaaagtggcccagagctgaaaaagccaggggaaacagtgaaaataagttgcaaggcatccggttacactttcacaaagtacggcatgaactgggtcaagcaggcccagggcaaggggctcaaatggatgggttggatcaataccaacactggcgagtctaggaatgctgaggagtttaagggccggtttgccttcagcctggagacaagtgccagcacagcttacctgcaaatcaacaatctgaagtatgaggatacagcaacctatttctgcggccgcggcactgtcgttgcagactggggacaaggtaccaccttgactgtatccagtgccagcactaagggcccatcagttttccccttggctccaagttctaaatccacaagcggtggaacagctgcactgggatgcctcgttaaagattatttccctgagcctgtgacagtgagctggaatagcggagcattgacttcaggtgtgcacacttttcccgctgtgttgcagtcctccggtctgtactcactgtccagtgtcgtaaccgtcccttctagcagcttgggaacccagacctacatctgtaacgtcaaccataaaccatccaacacaaaggtggataagaaggttgaaccaaagagctgtgataagacacatacatgccctccttgtcctgcaccagagctcctcggaggtccatctgtgttcctgtttccccccaaacccaaggacactcttatgatctctcgtactccagaggtcacctgtgttgttgtcgacgtgagccatgaagatcccgaggttaaattcaactggtacgtggatggagtcgaggttcacaatgccaagaccaagcccagggaggagcaatataattctacatatcgggtagtgagcgttctgaccgtgctccaccaagattggctcaatggaaaagagtacaagtgcaaggtgtccaacaaggctcttcccgctcccattgagaaaactatctccaaagccaaggggcagccacgggaaccccaggtgtatacattgcccccatctagagacgagctgaccaagaaccaggtgagtctcacttgtctggtcaaggggttttacccttctgacattgctgtagagtgggagtctaacggacagccagaaaacaactacaagacaactcccccagtgctggacagcgacgggagcttcttcctctactccaagttgactgtagacaagtctagatggcagcaaggaaacgttttctcctgctcagtaatgcatgaggctctgcacaatcactatacccagaaatcactgtcccttagcccagggtgactcgag

SEQ ID NO: 156 - DNA, 1424 bp, Artificial Sequence
  chCD37-38
aagcttgccaccatgggctggagttgtatcattctgtttttggtggccaccgccactggagtccattcccaagtgcaactccaggaatctggccctgacctggttaagccatctcagagcctctccctgacctgcactgttacaggatactcaatcacatcaggctttggctggcactggatcagacaatttcccgggaacaagttggaatggatggcttacattctgtatagcgggggtaccgattacaatccttccctcaagagccgaatctctatcaccagggatacaagcaagaaccaattttttctccgcctcagctctgtgactaccgaagataccgctacttactattgtgccaggggctactatggatatggtgcatggttcgtctattggggccagggaaccctggtgactgtgagcgctgcctctaccaagggcccatcagttttccccttggctccaagttctaaatccacaagcggtggaacagctgcactgggatgcctcgttaaagattatttccctgagcctgtgacagtgagctggaatagcggagcattgacttcaggtgtgcacacttttcccgctgtgttgcagtcctccggtctgtactcactgtccagtgtcgtaaccgtcccttctagcagcttgggaacccagacctacatctgtaacgtcaaccataaaccatccaacacaaaggtggataagaaggttgaaccaaagagctgtgataagacacatacatgccctccttgtcctgcaccagagctcctcggaggtccatctgtgttcctgtttccccccaaacccaaggacactcttatgatctctcgtactccagaggtcacctgtgttgttgtcgacgtgagccatgaagatcccgaggttaaattcaactggtacgtggatggagtcgaggttcacaatgccaagaccaagcccagggaggagcaatataattctacatatcgggtagtgagcgttctgaccgtgctccaccaagattggctcaatggaaaagagtacaagtgcaaggtgtccaacaaggctcttcccgctcccattgagaaaactatctccaaagccaaggggcagccacgggaaccccaggtgtatacattgcccccatctagagacgagctgaccaagaaccaggtgagtctcacttgtctggtcaaggggttttacccttctgacattgctgtagagtgggagtctaacggacagccagaaaacaactacaagacaactcccccagtgctggacagcgacgggagcttcttcctctactccaagttgactgtagacaagtctagatggcagcaaggaaacgttttctcctgctcagtaatgcatgaggctctgcacaatcactatacccagaaatcactgtcccttagcccagggtgactcga

SEQ ID NO: 157 - DNA, 1425 bp, Artificial Sequence
  huCD37-38
aagcttgccaccatgggttggagctgcatcattcttttcctggtcgctactgcaactggagtccactcacaggtccagctgcaagagtccggtcctgggcttgtgaaacccagccagtccctcagtctcacctgtactgtctctggctactctattaccagtgggttcggctggcattggattaggcagtttcccggtaaggggctggagtggatggcatatatcctgtacagcggaggaaccgattacaacccaagtctgaagagcaggatcagcattacccgggacacaagcaaaaaccagtttttccttcggctgtctagtgttacagctgcagacaccgctacttactattgtgctcggggttactatggctatggggcttggtttgtgtattggggacaaggcactcttgtgaccgtgagcagcgcctcaacaaagggcccatcagttttccccttggctccaagttctaaatccacaagcggtggaacagctgcactgggatgcctcgttaaagattatttccctgagcctgtgacagtgagctggaatagcggagcattgacttcaggtgtgcacacttttcccgctgtgttgcagtcctccggtctgtactcactgtccagtgtcgtaaccgtcccttctagcagcttgggaacccagacctacatctgtaacgtcaaccataaaccatccaacacaaaggtggataagaaggttgaaccaaagagctgtgataagacacatacatgccctccttgtcctgcaccagagctcctcggaggtccatctgtgttcctgtttccccccaaacccaaggacactcttatgatctctcgtactccagaggtcacctgtgttgttgtcgacgtgagccatgaagatcccgaggttaaattcaactggtacgtggatggagtcgaggttcacaatgccaagaccaagcccagggaggagcaatataattctacatatcgggtagtgagcgttctgaccgtgctccaccaagattggctcaatggaaaagagtacaagtgcaaggtgtccaacaaggctcttcccgctcccattgagaaaactatctccaaagccaaggggcagccacgggaaccccaggtgtatacattgcccccatctagagacgagctgaccaagaaccaggtgagtctcacttgtctggtcaaggggttttacccttctgacattgctgtagagtgggagtctaacggacagccagaaaacaactacaagacaactcccccagtgctggacagcgacgggagcttcttcctctactccaagttgactgtagacaagtctagatggcagcaaggaaacgttttctcctgctcagtaatgcatgaggctctgcacaatcactatacccagaaatcactgtcccttagcccagggtgactcgag

SEQ ID NO: 158 - DNA, 1425 bp, Artificial Sequence
  huCD37-50
aagcttgccaccatggggtggtcctgcataatccttttcctggttgctactgctaccggagtccattcacaggtgcagctgcaggagtccggccccggcctgctcaagccttctcagagtctgagtctgacttgtactgtttctggctacagcataaccagcggtttcgcttggcactggatcagacagcatcccggcaacaaactggagtggatgggatacatactgtactcaggctcaactgtctattccccctccctgaaatcccggatcagtattacccgtgacacttctaagaaccatttttttctgcagctgaacagcgttaccgcagctgacactgcaacctactactgtgcccggggatattatggatacggagcttggttcgcttactggggccaaggcaccctcgtaactgtgagtgctgcttccaccaagggcccatcagttttccccttggctccaagttctaaatccacaagcggtggaacagctgcactgggatgcctcgttaaagattatttccctgagcctgtgacagtgagctggaatagcggagcattgacttcaggtgtgcacacttttcccgctgtgttgcagtcctccggtctgtactcactgtccagtgtcgtaaccgtcccttctagcagcttgggaacccagacctacatctgtaacgtcaaccataaaccatccaacacaaaggtggataagaaggttgaaccaaagagctgtgataagacacatacatgccctccttgtcctgcaccagagctcctcggaggtccatctgtgttcctgtttccccccaaacccaaggacactcttatgatctctcgtactccagaggtcacctgtgttgttgtcgacgtgagccatgaagatcccgaggttaaattcaactggtacgtggatggagtcgaggttcacaatgccaagaccaagcccagggaggagcaatataattctacatatcgggtagtgagcgttctgaccgtgctccaccaagattggctcaatggaaaagagtacaagtgcaaggtgtccaacaaggctcttcccgctcccattgagaaaactatctccaaagccaaggggcagccacgggaaccccaggtgtatacattgcccccatctagagacgagctgaccaagaaccaggtgagtctcacttgtctggtcaaggggttttacccttctgacattgctgtagagtgggagtctaacggacagccagaaaacaactacaagacaactcccccagtgctggacagcgacgggagcttcttcctctactccaagttgactgtagacaagtctagatggcagcaaggaaacgttttctcctgctcagtaatgcatgaggctctgcacaatcactatacccagaaatcactgtcccttagcccagggtgactcgag

SEQ ID NO: 159 - DNA, 1425 bp, Artificial Sequence
  huCD37-51
aagcttgccaccatgggttggtcttgcatcatcctgttcctggtggccactgccactggcgtgcattcagaagttcagttggtggagtccggcccagaagtgctgaaacccggcgaatcactgtccctgacttgtaccgtgtcaggttatagcatcagcagcggctttgcttggcactggattcggcagtttccaggcaagggactggaatggatgggctacatccattacagtggctcaaccaattacagccctagcctgcagggccgaatctctattaccagggatagttctattaaccagtttttcctgcagcttaattccgtgactgcctctgacacagcaacttactattgcgcccgtggctactacgggttcggagcctggtttgtatactggggtcagggcaccctggtcactgtctcagccgcctctaccaagggcccatcagttttccccttggctccaagttctaaatccacaagcggtggaacagctgcactgggatgcctcgttaaagattatttccctgagcctgtgacagtgagctggaatagcggagcattgacttcaggtgtgcacacttttcccgctgtgttgcagtcctccggtctgtactcactgtccagtgtcgtaaccgtcccttctagcagcttgggaacccagacctacatctgtaacgtcaaccataaaccatccaacacaaaggtggataagaaggttgaaccaaagagctgtgataagacacatacatgccctccttgtcctgcaccagagctcctcggaggtccatctgtgttcctgtttccccccaaacccaaggacactcttatgatctctcgtactccagaggtcacctgtgttgttgtcgacgtgagccatgaagatcccgaggttaaattcaactggtacgtggatggagtcgaggttcacaatgccaagaccaagcccagggaggagcaatataattctacatatcgggtagtgagcgttctgaccgtgctccaccaagattggctcaatggaaaagagtacaagtgcaaggtgtccaacaaggctcttcccgctcccattgagaaaactatctccaaagccaaggggcagccacgggaaccccaggtgtatacattgcccccatctagagacgagctgaccaagaaccaggtgagtctcacttgtctggtcaaggggttttacccttctgacattgctgtagagtgggagtctaacggacagccagaaaacaactacaagacaactcccccagtgctggacagcgacgggagcttcttcctctactccaagttgactgtagacaagtctagatggcagcaaggaaacgttttctcctgctcagtaatgcatgaggctctgcacaatcactatacccagaaatcactgtcccttagcccagggtgactcgag

SEQ ID NO: 160 - DNA, 1425 bp, Artificial Sequence
  huCD37-56
aagcttgccaccatggggtggagctgcattatcctgttcctcgtcgccaccgcaaccggcgtccactcccaggtgcagctgcaagaaagcgggccaggattggtaaaaccttcccagtctctgagtcttacttgtaccgtatctggatacagtatcacatctggcttcgcctggcattggattcgccagtttcccggcaaggggcttgagtggatggggtatattcattattctggaggtaccaactacaacccttccctgaagagtcgagtctcaattaccagggacacttccaagaaccaattctttttgcagcttaattcagtgaccgctgccgacaccgctacttactactgcgcccggggctactatgggtttggtgcctggttcgcctactggggccaggggaccctggtgcccgtgtctgctgcctccacaaagggcccatcagttttccccttggctccaagttctaaatccacaagcggtggaacagctgcactgggatgcctcgttaaagattatttccctgagcctgtgacagtgagctggaatagcggagcattgacttcaggtgtgcacacttttcccgctgtgttgcagtcctccggtctgtactcactgtccagtgtcgtaaccgtcccttctagcagcttgggaacccagacctacatctgtaacgtcaaccataaaccatccaacacaaaggtggataagaaggttgaaccaaagagctgtgataagacacatacatgccctccttgtcctgcaccagagctcctcggaggtccatctgtgttcctgtttccccccaaacccaaggacactcttatgatctctcgtactccagaggtcacctgtgttgttgtcgacgtgagccatgaagatcccgaggttaaattcaactggtacgtggatggagtcgaggttcacaatgccaagaccaagcccagggaggagcaatataattctacatatcgggtagtgagcgttctgaccgtgctccaccaagattggctcaatggaaaagagtacaagtgcaaggtgtccaacaaggctcttcccgctcccattgagaaaactatctccaaagccaaggggcagccacgggaaccccaggtgtatacattgcccccatctagagacgagctgaccaagaaccaggtgagtctcacttgtctggtcaaggggttttacccttctgacattgctgtagagtgggagtctaacggacagccagaaaacaactacaagacaactcccccagtgctggacagcgacgggagcttcttcctctactccaagttgactgtagacaagtctagatggcagcaaggaaacgttttctcctgctcagtaatgcatgaggctctgcacaatcactatacccagaaatcactgtcccttagcccagggtgactcgag

SEQ ID NO: 161 - DNA, 1425 bp, Artificial Sequence
  huCD37-57
aagcttgccaccatgggctggagctgcatcattctgtttctggtggccacagcaactggcgttcacagtcaagtccaactgcaggagagcggccccggactcctgaaaccatctcagtcactcagtctgacatgtactgtgagcggctacagcattacctcaggcttcgcttggcattggatcaggcagttccccggaaaaggtctggagtggatggggtacattctgtacagcggcagtacagtgtattcaccctccttgaaatctaggatatcaatcacacgtgatacaagcaaaaatcagttcttcctccagctgaactccgtcaccgccgcagacacagcaacctattattgtgctcgcggatactacggatatggcgcatggttcgcctattggggccaggggacactcgtgaccgtttccgccgcctccacaaagggcccatcagttttccccttggctccaagttctaaatccacaagcggtggaacagctgcactgggatgcctcgttaaagattatttccctgagcctgtgacagtgagctggaatagcggagcattgacttcaggtgtgcacacttttcccgctgtgttgcagtcctccggtctgtactcactgtccagtgtcgtaaccgtcccttctagcagcttgggaacccagacctacatctgtaacgtcaaccataaaccatccaacacaaaggtggataagaaggttgaaccaaagagctgtgataagacacatacatgccctccttgtcctgcaccagagctcctcggaggtccatctgtgttcctgtttccccccaaacccaaggacactcttatgatctctcgtactccagaggtcacctgtgttgttgtcgacgtgagccatgaagatcccgaggttaaattcaactggtacgtggatggagtcgaggttcacaatgccaagaccaagcccagggaggagcaatataattctacatatcgggtagtgagcgttctgaccgtgctccaccaagattggctcaatggaaaagagtacaagtgcaaggtgtccaacaaggctcttcccgctcccattgagaaaactatctccaaagccaaggggcagccacgggaaccccaggtgtatacattgcccccatctagagacgagctgaccaagaaccaggtgagtctcacttgtctggtcaaggggttttacccttctgacattgctgtagagtgggagtctaacggacagccagaaaacaactacaagacaactcccccagtgctggacagcgacgggagcttcttcctctactccaagttgactgtagacaagtctagatggcagcaaggaaacgttttctcctgctcagtaatgcatgaggctctgcacaatcactatacccagaaatcactgtcccttagcccagggtgactcgag

SEQ ID NO: 162 - DNA, 717 bp, Artificial Sequence
  chCD37-3
gaattcgccaccatgagtgtgcccactcaggtcctggggttgctgctgctgtggcttacagatgccagatgtgacatccagatgactcagtctccagcctccctttctgtatctgtgggagaaactgtcaccatcacatgtcgagcaagtgagaatattcgcagtaatttagcatggtatcagcagaaacagggaaaatctcctcagctcctggtcaatgttgcaacaaacttagcagatggtgtgccatcaaggttcagtggcagtggatcaggcacacagtattccctcaagatcaacagcctgcagtctgaagattttgggacttattactgtcaacattattggggtactacgtggacgttcggtggaggcaccaagctggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 163 - DNA, 714 bp, Artificial Sequence
  huCD37-3 (1.0 and 1.1)
gaattcgccaccatgggttggtcctgcatcatcttgtttctcgtggccacagccaccggtgttcactctgatatacaaatgactcaaagcccttccagtttgagcgtaagtgtgggtgaacgcgtaacaatcacctgtagagctagtgaaaacatccgcagtaatctcgcatggtaccaacaaaagccaggtaagtcacctaagctcctcgtgaatgttgctaccaacctcgctgatggtgtgccttcacgattctctggttcaggttccggtaccgattattcacttaagatcaactcactccaaccagaagatttcggtacatattactgtcaacactactggggtacgacctggacattcggtcaaggtactaagctggaaatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 164 - DNA, 726 bp, Artificial Sequence
  chCD37-12
gaattcgccaccatgggttggtcctgtataatcctgttcttggtggccaccgctactggcgttcatagtgatattgtactcactcagtcaccagccagtctggcagtgtccctgggccagcgtgccaccatctcctgccgggcctcacagtccgtgagcactagctcttattcctatctctactggtttcaacagaagccaggacagccccctaagctgctgatcaagtacgcctccaacctcgccagcggcgttcccgctagattctctggttccggtagcggaactgatttcactttgaacatccaccccgttgaggaagaggataccgccacttactattgtcaacactcttgggagattccttacacctttggaggaggaacaaagctcgaaattaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 165 - DNA, 711 bp, Artificial Sequence
  chCD37-38
gaattcgccaccatgggctggtcctgtatcatcctgtttctcgtggccacagctacaggtgttcattctcagattgtgctgacccaatcaccagctattatgtccgctagccccggcgagaaagtgacaatgacatgtagcgctagctcttctgtgacttacatgcattggtatcaacagaagtcaggtaccagtcccaagcgttggatctacgacacatccaaactggcctccggagtccctgccaggttcagcggaggtgggtccggcaccagttattcactgaccatatcctctatggaagctgaagatgctgctacttattattgtcaacaatggatttctaacccccccacctttggtggcggaacaaagctggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 166 - DNA, 711 bp, Artificial Sequence
  huCD37-38
gaattcgccaccatgggatggtcctgcattattctgttcttggtcgccactgctactggcgttcactctgacattgtgctcacacagtctccagcctcaatgtctgcttcccccggtgagcgggtgaccatgacatgctctgccagttcctccgtgacatatatgcattggtatcagcaaaaacccggtacctctccaaaaagatggatctacgacacttcaaagcttgcatcaggcgttcctgccagattttccgggtctgggtctggcacttcatacagtctgaccattagttccatggaagctgaagatgcagccacctattactgtcagcagtggatttcaaatcctcctaccttcggcggcggaaccaaactggagataaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 167 - DNA, 711 bp, Artificial Sequence
  huCD37-50
gaattcgccaccatgggttggtcatgcattattctgttcctggttgctaccgcaacaggagtacatagtgagatagtcctcacccaaagtcctgctactatgtctgccagcccaggagagcgtgtgaccatgacttgctctgcaacctcaagtgtgacatacatgcattggtatcagcaaaagcctggccaatcccctaaaaggtggatctacgatacttctaatctgccatacggtgtgcccgcaaggttctccgggagtggcagtggcaccagttatagtctgaccatcagttcaatggaagcagaggatgcagcaacctattattgtcagcagtggtccgataatccccctacttttggtcagggtacaaagctggagattaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 168 - DNA, 711 bp, Artificial Sequence
  huCD37-51
gaattcgccaccatgggatggagctgtattattctgttcctggttgctactgctactggcgtccattccgagatagtcctcacccagagccccgcaaccatgagtgcctcccctggggagcgagtgactatgacttgttccgccacttcttcagttacctatatgcattggtatcagcagaaacctggacagtctccaaagcgttggatttacgacacctccaacctggcttcaggagttcctgctaggttcagcggatctgggtctggcacaagttattcactcaccattagttccatggaggccgaagatgccgctacttactactgtcagcagtggagcagcaacccccctacattcgggcagggaactaagctggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 169 - DNA, 711 bp, Artificial Sequence
  huCD37-56
gaattcgccaccatgggctggtcctgtatcatcctgtttctggtggcaaccgctactggggttcactctgatattgtcctgacacagagtccagccttcatgagtgcttctcccggagaaaaggtcacaatgacttgttcagcttcctcctccgtcacatacatgcattggtaccagcagaagcctgaccagagtcctaagaggtggatctatgatacaagcaatctggcttccggtgtcccctcccgcttttcaggcggcggaagcggaactgactatagccttaccatctcctcaatggaagccgaggacgctgctacatattactgccagcaatggatcagcgaccctcctactttcggacagggaacaaaattggaaattaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 170 - DNA, 711 bp, Artificial Sequence
  huCD37-57
gaattcgccaccatggggtggtcctgtattatcctgttcctggtcgcaaccgccacaggcgttcactccgagatcgtgttgactcagagcccagccaccatgtccgcttcccccggggagagagtgacaatgacttgttccgccacaagttctgtaacctacatgcattggtaccagcaaaaaccaggacagagtccccgtcgttggatttatgatacctctaacctggcttcaggcgttcctgcccgcttttctggtagtggatctgggacttcctatagccttaccataagctctatggaagccgaggacgccgctacatactactgccagcagtggagtgataacccccccaccttcgggcagggaaccaaattggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

(19) United States
(12) Patent Application Publication
(10) Pub. No.: US 2015/0093397 A1
(43) Pub. Date: Apr. 2, 2015

(54) Methods for Increasing Efficacy of CD37-Based Therapy

(71) Applicant: ImmunoGen, Inc., Waltham, MA (US)

(72) Inventor: Christina N. Carrigan, Belmont, MA (US)

(21) Appl. No.: 14/389,727

(22) PCT Filed: Mar. 29, 2013
(86) PCT No.: PCT/US2013/034646

§ 371 Date: Sep. 30, 2014

Related U.S. Application Data
(60) Provisional application No. 61/618,489, filed on Mar. 30, 2012.

(51) Int. Cl. G01N 33/574; C07K 16/28

(52) U.S. Cl. CPC G01N 33/574; C07K 16/2896; C07K 2317/76; G01N 2333/70596
USPC 424/172.1; 424/178.1; 435/29; 435/7.23; 435/6.12; 435/6.11; 530/389.7; 530/391.1; 530/391.7; 530/387.3; 530/391.9; 506/9

(57) ABSTRACT

Methods to improve the success of cancer therapies that target CD37 are provided. Kits comprising reagent useful in the methods are further provided.

### FIELD OF THE INVENTION

The field of invention generally relates to increasing the efficacy of the treatment of B-cell diseases characterized by the overexpression of human CD37. More specifically, the invention concerns more effective treatment of patients susceptible to or diagnosed with B-cell diseases, for example cancer or autoimmune disease, in which the cells express CD37 as determined by a gene expression assay, with a CD37 antagonist, e.g., a CD37 immunoconjugate.

### BACKGROUND OF THE INVENTION

Cancer is one of the leading causes of death in the developed world, with over one million people diagnosed with cancer and 500,000 deaths per year in the United States alone. Overall it is estimated that more than 1 in 3 people will develop some form of cancer during their lifetime.

Leukocyte antigen CD37 (“CD37”), also known as GP52-40, tetraspanin-26, or TSPAN26, is a transmembrane protein of the tetraspanin superfamily (Maecker et al., 1997 FASEB J. 11:428-442). It is a heavily glycosylated protein with four transmembrane domains that is expressed on B cells during the pre-B to peripheral mature B-cell stages, but is absent on terminal differentiation to plasma cells. (Link et al., 1987, J Pathol. 152:12-21). The CD37 antigen is only weakly expressed on T-cells, myeloid cells and granulocytes (Schwartz-Albiez et al. 1988, J. Immunol., 140(3)905-914). However, CD37 is also expressed on malignant B-cells such as those founding non-Hodgkin's lymphoma (NHL) and chronic lymphoid leukemia (CLL) (Moore et al. 1986, J Immunol. 137(9):3013-8). This expression profile suggests that CD37 represents a promising therapeutic target for B-cell malignancies, and currently, there is a clear unmet medical need for more effective therapeutics for B-cell malignancies.

### SUMMARY OF THE INVENTION

The present invention is based on the discovery of a dynamic range of expression of CD37 in cancer and the discovery that cancers with increased levels of CD37 expression are more responsive to treatment with anti-CD37 antibodies or anti-CD37 immunoconjugates. The present invention advantageously permits treatment of patients who have a greater likelihood of responding to treatment by administering therapeutic agents, i.e., anti-CD37 antibodies or anti-CD37 immunoconjugates, to patients who are found to have an increased expression level of CD37.

In one embodiment, the invention provides a method for increasing the efficacy of cancer therapy with an anti-CD37 antibody, or anti-CD37 immunoconjugate, wherein the method comprises administering to a subject having cancer an anti-CD37 antibody, or anti-CD37 immunoconjugate, wherein an increased expression of CD37 gene or protein in a cancerous sample from the subject has been detected using a detection method that distinguishes between staining intensity or staining uniformity in a CD37 expressing cancerous sample as compared to staining intensity or staining uniformity in one or more reference sample.

In another embodiment, the invention provides a method of identifying a cancer as likely sensitive to treatment with an anti-CD37 antibody, or anti-CD37 immunoconjugate comprising (a) measuring the level of CD37 expression in a cancerous sample obtained from the subject cancer, wherein the measuring comprises the use of a detection method that distinguishes between staining intensity or staining uniformity in a CD37 expressing cancerous sample as compared to staining intensity or staining uniformity in one or more reference samples; (b) determining a CD37 staining intensity or staining uniformity score for the cancerous, sample; and (c) comparing the CD37 staining intensity or staining uniformity score determined in step (b) to a relative value determined by measuring CD37 protein expression in at least one reference sample, wherein the at least one reference sample is a tissue, cell, or cell pellet sample which is not sensitive to treatment with an anti-CD37 antibody, or anti-CD37 immunoconjugate, and wherein a CD37 staining intensity score for the sample determined in step (b) that is higher than the relative value identifies the cancer as sensitive to treatment with an anti-CD37 antibody, or anti-CD37 immunoconjugate.

In another embodiment, the invention provides a method for identifying a cancer likely to respond to an anti-CD37 antibody, or anti-CD37 immunoconjugate comprising (a) contacting a biological sample comprising cells from the cancer with an agent that binds CD37 protein on the cell surface; (b) detecting binding of the agent that binds CD37 protein on the cell surface of the biological sample of (a); (c) assigning a score to the binding of step (b), wherein the score is assigned based on comparison to one or more reference samples; and (d) comparing the score in step (c) to the score of a reference tissue or cell, wherein a score for the cancer CD37 level that is greater than the score for a negative or low CD37 expressing reference sample or a score for the cancer CD37 level that is equal to or greater than the score for a high CD37 expressing reference sample identifies the cancer as likely to respond to an anti-CD37 antibody or anti-CD immunoconjugate.

In another embodiment, the invention provides a method of identifying a subject having a cancer as likely to respond to a low dose anti-CD37 antibody or anti-CD37 immunoconjugate treatment regimen comprising: (a) contacting a biological sample comprising cells from the cancer with an agent that binds cell surface CD37 protein; (b) detecting binding of the agent to the biological sample of (a); (c) assigning a score to the binding of step (b), wherein the score is assigned based on comparison to one or more reference samples; and (d) comparing the score in step (c) to the score of a reference tissue or cell, wherein a score for the cancer CD37 level that is greater than the score for a negative or low CD37 expressing reference sample or a score for the cancer CD37 level that is equal to or greater than the score for a high CD37 expressing reference sample identifies the cancer as likely to respond to a low dose anti-CD37 antibody or anti-CD37 immunoconjugate.

In another embodiment, the invention provides a method of optimizing a therapeutic regimen with an anti-CD37 antibody or an anti-CD37 immunoconjugate for a subject having cancer comprising: (a) detecting the level of CD37 expression in a cancerous sample obtained from the subject; (b) comparing the level of CD37 expression in the cancerous sample to the CD37 expression in a reference sample; (c) determining a CD37 staining intensity score for the cancerous sample; and (d) administering an increased dose of an anti-CD37 antibody or an anti-CD37 immunoconjugate to the subject if the score is low or administering a decreased dose of an anti-CD37 antibody or an anti-CD37 immunoconjugate to the subject if the score is high.

In another embodiment, the invention provides a method of detecting the expression of cell surface CD37 on cancer cells in a cancerous sample from a subject comprising: (a) obtaining a cancerous sample, wherein the sample is formalin-fixed paraffin embedded; (b) contacting the cancerous sample with an antibody that specifically binds cell surface CD37; (c) measuring the binding of the antibody in (b) to the cell surface CD37 in the cancerous sample using a detection method that can distinguish between staining intensity or staining uniformity in a CD37 expressing sample as compared to staining intensity or staining uniformity in one or more reference samples; and (d) assigning a CD37 expression score to the CD37 after comparing the level of cell surface CD37 staining intensity or staining uniformity in the cancerous sample to one or more reference samples.

In some embodiments, detection is by immunohistochemistry (IHC). The IHC can be calibrated IHC that can distinguish different levels of CD37 expression.

In some embodiments, the detection method produces a range of staining intensity for samples having low cell surface CD37 expression, intermediate CD37 cell surface expression, or high CD37 cell surface expression.

In some embodiments, the detection method distinguishes between staining intensity and staining uniformity in a CD37 expressing cancerous sample as compared to a reference sample.

In some embodiments, the cancerous sample or biological sample has a staining intensity score greater than 1 for CD37 expression by immunohistochemistry. In some embodiments, the cancerous sample or biological sample has a staining intensity score of 2, 3, or 3+ for CD37 expression by immunohistochemistry. In some embodiments, the cancerous sample or biological sample has a staining intensity score of 2, 3, or 3+ for CD37 expression by immunohistochemistry on a formalin fixed paraffin embedded sample. In some embodiments, the cancerous sample or biological sample has a staining uniformity for CD37 expression that is homogenous. In some embodiments, the cancerous sample or biological sample has a staining intensity score of 2, 3, or 3+ for CD37 and a staining uniformity that is heterogeneous or homogenous.

In some embodiments, the immunohistochemistry is performed manually. In some embodiments, the immunohistochemistry is performed using an automated system.

In some embodiments, the reference sample is a positive reference sample or a negative reference sample. In some embodiments, the reference sample comprises cells, cell pellets, or tissue.

In some embodiments, the detection comprises detecting CD37 expression with an antibody that specifically binds cell surface CD37. In some embodiments, the antibody further comprises a detection reagent selected from the group consisting of an enzyme, a fluorophore, a radioactive label, and a luminophore. In some embodiments, the detection reagent is selected from the group consisting of biotin, digoxigenin, fluorescein, tritium, and rhodamine. In some embodiments, the antibody is clone CT1.

In some embodiments, the concentration of the antibody is about 1 to about 10 μg/mL or about 2.1 to about 8.4 μg/mL, about 4 to about 5 μg/mL, or 2.1, 4.2, or 8.4 μg/mL. In one embodiment the concentration of the antibody (e.g., clone CT1) is 4.2 μg/mL.

In some embodiments, the cancer is selected from the group consisting of B cell lymphomas, NHL, precursor B cell lymphoblastic leukemia/lymphoma and mature B cell neoplasms, B cell chronic lymphocytic leukemia (CLL)/small lymphocytic lymphoma (SLL), B cell prolymphocytic leukemia, lymphoplasmacytic lymphoma, mantle cell lymphoma (MCL), follicular lymphoma (FL), low grade, intermediate-grade and high-grade (FL), cutaneous follicle center lymphoma, marginal zone B cell lymphoma, MALT type marginal zone B cell lymphoma, nodal marginal zone B cell lymphoma, splenic type marginal zone B cell lymphoma, hairy cell leukemia, diffuse large B cell lymphoma (DLBCL), Burkitt's lymphoma (BL), plasmacytoma, plasma cell myeloma, post-transplant lymphoproliferative disorder, Waldenstrom's macroglobulinemia, and anaplastic large-cell lymphoma (ALCL).

The present invention also provides articles of manufacture comprising an anti-CD37 antibody or an anti-CD37 immunoconjugate, a container, and a package insert or label indicating that the antibody or immunoconjugate can be used to treat a cancer characterized by the expression of CD37 at a level of 2, 3, or 3+ measured by IHC.

The present invention also provides combination diagnostic and pharmaceutical kits comprising a murine anti-CD37 antibody for use in diagnosis and an anti-CD37 antibody or an anti-CD37 immunoconjugate for use in therapy. In some embodiments, the diagnostic antibody is a detection antibody that is able to detect CD37 expression by IHC.

The present invention also provides diagnostic kits comprising a detection antibody that can specifically bind to cell surface CD37, a reagent for immunohistochemistry (IHC), and one or more standardized reference samples, wherein the standardized reference samples comprise formalin fixed paraffin embedded cells, cell pellets, or tissue samples, and wherein the one or more standardized referenced samples are from non-CD37 expressing, low-CD37 expressing, intermediate-CD37 expressing, or high CD37 expressing cells, cell pellets, or tissues. Examples of such reference samples are described herein in the Examples.

In some embodiments, the article or kit is one in which the IHC is calibrated IHC that can distinguish different levels of CD37 expression. In some embodiments, the calibrated IHC produces a range of staining intensity for samples having low cell surface CD37 expression, intermediate cell surface CD37 expression, or high cell surface CD37 expression. In some embodiments, the IHC distinguishes between staining intensity and staining uniformity in a CD37 expressing sample as compared to a reference sample. In some embodiments, the IHC is performed on a formalin fixed paraffin embedded sample. The IHC can be performed manually or performed using an automated system.

In some embodiments, the article or kit comprises a CD37 immunoconjugate comprising an anti-CD37 antibody, a linker, and a cytotoxin. In some embodiments, the anti-CD37 antibody is chimeric, or humanized CD37-3, CD37-38, or CD37-50. In some embodiments, the anti-CD37 antibody is an antibody selected from the group consisting of an antibody comprising the polypeptide of SEQ ID NO:56 and the polypeptide of SEQ ID NO:73, an antibody comprising the polypeptide of SEQ ID NO:57 and the polypeptide of SEQ ID NO:74, an antibody comprising the polypeptide of SEQ ID NO:58 and the polypeptide of SEQ ID NO:74, an antibody comprising the polypeptide of SEQ ID NO:62 and the polypeptide of SEQ ID NO:78, an antibody comprising the polypeptide of SEQ ID NO:63 and the polypeptide of SEQ ID NO:79, and an antibody comprising the polypeptide of SEQ ID NO:65 and the polypeptide of SEQ ID NO:81.

In some embodiments, the linker is selected from the group consisting of a cleavable linker, a non-cleavable linker, a hydrophilic linker, and a dicarboxylic acid based linker. In some embodiments, the linker is selected from the group consisting: N-succinimidyl 4-(2-pyridyldithio)pentanoate (SPP) or N-succinimidyl 4-(2-pyridyldithio)-2-sulfopentanoate (sulfo-SPP); N-succinimidyl 4-(2-pyridyldithio)butanoate (SPDB) or N-succinimidyl 4-(2-pyridyldithio)-2-sulfobutanoate (sulfo-SPDB); N-succinimidyl 4-(maleimidomethyl) cyclohexanecarboxylate (SMCC); N-sulfosuccinimidyl 4-(maleimidomethyl) cyclohexanecarboxylate (sulfoSMCC); N-succinimidyl-4-(iodoacetyl)-aminobenzoate (SIAB); and N-succinimidyl-[(N-maleimidopropionamido)-tetraethyleneglycol]ester (NHS-PEG4-maleimide). In some embodiments, the linker is N-succinimidyl 4-(maleimidomethyl) cyclohexanecarboxylate (SMCC). In some embodiments, the cytotoxin is selected from the group consisting of a maytansinoid, maytansinoid analog, benzodiazepine, taxoid, CC-1065, CC-1065 analog, duocarmycin, duocarmycin analog, calicheamicin, dolastatin, dolastatin analog, auristatin, tomaymycin derivative, and leptomycin derivative or a prodrug of the cytotoxin. In some embodiments, the cytotoxin is a maytansinoid. In some embodiments, the maytansinoid is N(2′)-deacetyl-N(2′)-(3-mercapto-1-oxopropyl)-maytansine or N(2′)-deacetyl-N2-(4-mercapto-4-methyl-1-oxopentyl)-maytansine. In some embodiments, the maytansinoid is is N(2′)-deacetyl-N(2′)-(3-mercapto-1-oxopropyl)-maytansine (DM1). In some embodiments, the immunoconjugate comprises the antibody huCD37-3, SMCC, and DM1.

In some embodiments, a combination diagnostic and pharmaceutical kit further comprises one or more reference samples. In some embodiments, the reference sample is a positive reference sample or a negative reference sample. In some embodiments, the reference sample comprises cells, cell pellets, or tissue.

In some embodiments, a detection antibody for use in kit as provided herein further comprises a detection reagent selected from the group consisting of: an enzyme, a fluorophore, a radioactive label, and a luminophore. In some embodiments, the detection reagent is selected from the group consisting of biotin, digoxigenin, fluorescein, tritium, and rhodamine. The concentration of antibody in a kit can be about 1 to about 10 μg/mL or about 2.1 to about 8.4 μg/mL, about 4 to about 5 μg/mL, or 2.1, 4.2, or 8.4 μg/mL or 4.2 μg/mL.

In some embodiments, a kit comprises a low-CD37 expressing control, and the low-CD37 expressing control is Namalwa or RL tumor cells. In some embodiments, a kit comprises a high-CD37 expressing control, and the high-CD37 expressing control is selected form the group consisting of: Daudi, and Ramos cell lines, and a cell line stably or transiently transfected with CD37. In some embodiments, the cell line stably or transiently transfected with CD37 is 300-19/CD37.

In some embodiments, the anti-CD37 antibody or immunoconjugate is an antibody or immunoconjugate as described in International Published Application Nos. WO 2011/112978 or WO 2012/135740, each of which is herein incorporated by reference in its entirety.

### BRIEF DESCRIPTIONS OF THE DRAWINGS

FIG. 1. Immunohistochemical (IHC) staining of CD37 on non-transfected 300-19 cells (left) and 300-19 cells transfected with human CD37 (huCD37).

FIG. 2. IHC staining of CD37 in white pulp of human spleen tissue at 10× (left) and 40× (right) magnification.

FIG. 3. IHC staining results for CD37 obtained with the optimized manual method for Daudi, Ramos and RL cells. CD37 expression levels obtained by quantitative flow cytometry are given below each image.

FIG. 4. Flow cytometry histograms obtained after staining with a PE-labeled anti-CD37 antibody (bold lines) in comparison to unstained samples (thin lines) using Daudi, Ramos and RL cells.

FIG. 5. IHC staining results for CD37 on Daudi cells and for CD20 on RL cells obtained with the optimized manual method. Cells expressing similar CD37 and CD20 antigen levels show a similar staining pattern.

FIG. 6. Summary of staining scores for CD37 obtained with the optimized manual method for lymphoma samples in comparison to staining scores for CD20.

FIG. 7, Representative pictures of NHL patient samples stained for CD37 using the optimized manual method.

FIG. 8. Anti-tumor activity (median tumor volume, mm³) of huCD37-3-SMCC-DM1 in female CB.17 SCID mice bearing SU-DHL-4 xenografts.

FIG. 9. Anti-tumor activity (median tumor volume, mm³) of huCD37-3 antibody, huCD37-3-SMCC-DM1, and standard chemotherapeutics in SCID mice bearing DoHH2 human tumor xenografts.

FIG. 10. Anti-tumor activity (median tumor volume, mm³) huCD37-3-SMCC-DM1 in SCID mice bearing DoHH2 human tumor xenografts.

FIG. 11. Anti-tumor activity (median tumor volume, mm³) of huCD37-3 antibody, huCD37-3-SMCC-DM1, Ofatumumab, and Bendamustine in SCID mice bearing JVM-3 human tumor xenografts.

FIG. 12. Anti-tumor activity (median tumor volume, mm³) of huCD37-3-SMCC-DM1 in SCID mice bearing JVM-3 human tumor xenografts.

FIG. 13. Scoring distribution in large cell lymphoma (DLBCL) using automated staining methods.

FIG. 14. Scoring distribution in small cell lymphoma including follicular lymphoma (FL). Mantle cell lymphoma (MCL), MALT type marginal zone B cell lymphoma, marginal zone B cell lymphoma, unclassified small cell lymphoma, and unclassified non-Hodgkin's lymphoma (NHL) samples using automated staining methods.

### DETAILED DESCRIPTION OF THE INVENTION

The present invention provides methods for treating B-cell diseases such as cancer and autoimmune diseases and for increasing the efficacy of or the likelihood of response to the treatment diseases characterized by the overexpression of CD37. The present invention is based on the discovery of methods of detecting a dynamic range of expression of CD37 in a cancer and autoimmune diseases as compared to negative normal tissue and the discovery that B-cells with high levels of CD37 expression are more responsive to treatment with anti-CD37 antibodies or anti-CD37 immunoconjugates. Kits comprising one or more reagents useful for practicing the methods of the invention are further provided.

### I. Definitions

To facilitate an understanding of the present invention, a number of terms and phrases are defined below.

The term CD37 as used herein, refers to any native CD37, unless otherwise indicated. CD37 is also referred to as GP52-40, leukocyte antigen CD37, and Tetraspanin-26. The term “CD37” encompasses “full-length,” unprocessed CD37 as well as any form of CD37 that results from processing in the cell. The term also encompasses naturally occurring variants of CD37, e.g., splice variants, allelic variants, and isoforms. The CD37 polypeptides described herein can be isolated from a variety of sources, such as from human biological samples or from another source, or prepared by recombinant or synthetic methods.

The term “increased expression” or “high expression” of CD37 refers to a sample which contains elevated levels of CD37 expression as compared to a negative or low reference control or as compared to a healthy or non-diseased sample of the same tissue or cell type. In one example, the CD37 expression is measured by IHC and given a staining intensity score or a staining uniformity score by comparison to calibrated controls exhibiting defined scores (e.g., an intensity score of 3 is given to the test sample if the intensity is comparable to the level 3 calibrated control or an intensity of 2 is given to the test sample if the intensity is comparable to the level 2 calibrated control). For example, a score of 1, 2, 3, or 3+ or greater, preferrably a score of 2, 3, 3+, or greater, by immunohistochemistry indicates increased expression of CD37. A staining uniformity that is heterogeneous or homogeneous is also indicative of increased CD37 expression. The staining intensity and staining uniformity scores can be used alone or in combination (e.g., 2 homo, 2 hetero, 3 homo, 3 hetero, etc.). In another example, an increase in CD37 expression can be determined by detection of an increase of at least 2-fold, at least 3-fold, or at least 5-fold relative to control values (e.g., expression level in a biological sample, tissue, or cell from a subject without cancer or with a cancer that does not have elevated CD37 values).

The term “overexpression” of CD37 in a particular tumor, tissue, or cell sample refers to CD37 (a CD37 polypeptide or a nucleic acid encoding such a polyptpide) that is present at a level higher than that which is present in non-diseased tissue or cells of the same type or origin or other cells in the proximity of a tumor or cancer. Such overexpression can be caused, for example, by mutation, gene amplification, increased transcription, or increased translation.

A “reference sample” can be used to correlate and compare the results obtained in the methods of the invention from a test sample. Reference samples can be cells (e.g., cell lines, cell pellets) or tissue. The CD37 levels in the “reference sample” can be an absolute or relative amount, a range of amount, a minimum and/or maximum amount, a mean amount, and/or a median amount of CD37. The diagnostic methods of the invention involve a comparison between expression levels of CD37 in a test sample and a “reference value.” In some embodiments, the reference value is the expression level of the CD37 in a reference sample. A reference value can be a predetermined value and can also be determined from reference samples (e.g., control biological samples) tested in parallel with the test samples. A reference value can be a single cut-off value, such as a median or mean or a range of values, such as a confidence interval. Reference values can be established for various subgroups of individuals, such as individuals predisposed to cancer, individuals having early or late stage cancer, male and/or female individuals, or individuals undergoing cancer therapy. Examples of negative reference samples or values and positive reference samples or values are described herein.

In some embodiments, the reference sample is a sample from a healthy tissue, in particular a corresponding tissue which is not affected by cancer. These types of reference samples are referred to as negative control samples or reference samples. In other embodiments, the reference sample is a sample from a tumor that expresses CD37. These types of reference samples are referred to as positive control samples. Positive control samples can also be used as a comparative indicator for the type (hetero versus homo) and/or degree (0, 1, 2, 3, 3+) of staining intensity, which correlates with the level of CD37 expression. Positive control comparative samples are also referred to as calibrated reference samples. As shown in the Examples, low or non-CD37 references include the red pulp of the spleen (e.g., monocytes and red blood cells) and T-cells and high CD37-expressing references include white pulp of the spleen (e.g., B lymphocytes). For cell lines, exemplary non-expressors include 300-19 cells, and high expressors include Daudi, Ramos, and RL cells. Another positive high CD37 reference is a cell line stably or transiently transfected with CD37 (e.g., 300-19/CD37). Appropriate positive and negative reference levels of CD37 for a particular cancer can be determined by measuring levels of CD37 in one or more appropriate subjects, and such reference levels may be tailored to specific populations of subjects (e.g., a reference level may be age-matched so that comparisons may be made between CD37 levels in samples from subjects of a certain age and reference levels for a particular disease state, phenotype, or lack thereof in a certain age group). Such reference levels may also be tailored to specific techniques that are used to measure levels of CD37 in biological samples (e.g., immunoassays, etc.), where the levels of CD37 may differ based on the specific technique that is used.

The term “primary antibody” herein refers to an antibody that binds specifically to the target protein antigen in a sample. A primary antibody is generally the first antibody used in an immunohistochemical (IHC) procedure. In one embodiment, the primary antibody is the only antibody used in an IHC procedure. The term “secondary antibody” herein refers to an antibody that binds specifically to a primary antibody, thereby forming a bridge between the primary antibody and a subsequent reagent, if any. The secondary antibody is generally the second antibody used in an immunohistochemical procedure. The term “tertiary antibody” herein refers to an antibody that binds specifically to a secondary antibody, thereby forming a bridge between the secondary antibody and a subsequent reagent, if any.

A “sample” of the present invention is of biological origin, in specific embodiments, such as from eukaryotic organisms. In preferred embodiments, the sample is a human sample, but animal samples may also be used in the practice of the invention. Non-limiting sources of a sample for use in the present invention include solid tissue, biopsy aspirates, fluidic extracts, blood, plasma, serum, spinal fluid, lymph fluid, the external sections of the skin, respiratory, intestinal, and genitourinary tracts, tears, saliva, milk, tumors, organs, cell cultures and/or cell culture constituents, for example. A “cancerous sample” is a sample that contains a cancerous cell. The present invention is useful for solid tissue samples where the amount of available material is small. The method can be used to examine an aspect of expression of CD37 or a state of a sample, including, but not limited to, comparing different types of cells or tissues, comparing different developmental stages, and detecting or determining the presence and/or type of disease or abnormality.

For the purposes herein, a “section” of a tissue sample refers to a single part or piece of a tissue sample, e.g., a thin slice of tissue or cells cut from a tissue sample. It is understood that multiple sections of tissue samples may be taken and subjected to analysis according to the present invention. In some cases, the selected portion or section of tissue comprises a homogeneous population of cells. In other cases, the selected portion comprises a region of tissue, e.g., the lumen as a non-limiting example. The selected portion can be as small as one cell or two cells, or could represent many thousands of cells, for example. In most cases, the collection of cells is important, and while the invention has been described for use in the detection of cellular components, the method may also be used for detecting non-cellular components of an organism (e.g., soluble components in the blood as a non-limiting example).

By “correlate” or “correlating” is meant comparing, in any way, the performance and/or results of a first analysis with the performance and/or results of a second analysis. For example, one may use the results of a first analysis in carrying out the second analysis and/or one may use the results of a first analysis to determine whether a second analysis should be performed and/or one may compare the results of a first analysis with the results of a second analysis. In one embodiment, increased expression of CD37 correlates with increased likelihood of effectiveness of a CD37-targeting anti-cancer therapy.

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, antibody fragments (such as Fab, Fab′, F(ab′)2, and Fv fragments), single chain Fv (scFv) mutants, multispecific antibodies such as bispecific antibodies generated from at least two intact antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. An antibody can be of any the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc.

A “blocking” antibody or an “antagonist” antibody is one which inhibits or reduces biological activity of the antigen it binds, such as CD37, In a certain embodiment blocking antibodies or antagonist antibodies substantially or completely inhibit the biological activity of the antigen. Desirably, the biological activity is reduced by 10%, 20%, 30%, 50%, 70%, 80%, 90%, 95%, or even 100%.

The term “anti-CD37 antibody” or “an antibody that binds to CD37” refers to an antibody that is capable of binding CD37 with sufficient affinity such that the antibody is useful as a diagnostic and/or therapeutic, agent in targeting CD37. The extent of binding of an anti-CD37 antibody to an unrelated, non-CD37 protein is less than about 10% of the binding of the antibody to CD37 as measured, e.g., by a radioimmunoassay (RIA). In certain embodiments, an antibody that binds to CD37 has a dissociation constant (Kd) of ≦1 μM, ≦100 nM, ≦10 nM, ≦1 nM, or ≦0.1 nM. Examples of anti-CD37 antibodies are known in the art and are disclosed in U.S. Published Application No 2011/0256153, which is herein incorporated by reference.

The term “antibody fragment” refers to a portion of an intact antibody and refers to the antigenic determining variable regions of an intact antibody. Examples of antibody fragments include, but are not limited to Fab, Fab′, F(ab′)2, and Fv fragments, linear antibodies, single chain antibodies, and multispecific antibodies formed from antibody fragments.

A “monoclonal antibody” refers to a homogeneous antibody population involved in the highly specific recognition and binding of a single antigenic determinant, or epitope. This is in contrast to polyclonal antibodies that typically include different antibodies directed against different antigenic determinants. The term “monoclonal antibody” encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal antibody” refers to such antibodies made in any number of manners including but not limited to by hybridoma, phage selection, recombinant expression, and transgenic animals.

The term “epitope” or “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids are typically retained upon protein denaturing, whereas epitopes formed by tertiary folding are typically lost upon protein denaturing. An epitope typically includes at least 3, and more usually, at least 5 or 8-10 amino acids in a unique spatial conformation.

“Binding affinity” generally refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention. Specific illustrative embodiments are described in the following.

“Or better” when used herein to refer to binding affinity refers to a stronger binding between a molecule and its binding partner. “Or better” when used herein refers to a stronger binding, represented by a smaller numerical Kd value. For example, an antibody which has an affinity for an antigen of “0.6 nM or better”, the antibody's affinity for the antigen is <0.6 nM, i.e., 0.59 nM, 0.58 nM, 0.57 nM etc. or any value less than 0.6 nM.

The phrase “substantially similar,” or “substantially the same”, as used herein, denotes a sufficiently high degree of similarity between two numeric values (generally one associated with an antibody of the invention and the other associated with a reference/comparator antibody) such that one of skill in the art would consider the difference between the two values to be of little or no biological and/or statistical significance within the context of the biological characteristics measured by said values (e.g., Kd values). The difference between said two values is less than about 50%, less than about 40%, less than about 30%, less than about 20%, or less than about 10% as a function of the value for the reference/comparator antibody.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cell or compositions include those which have been purified to a degree that they are no longer in a form in which they are found in nature. In some embodiments, an antibody, polynucleotide, vector, cell, or composition which is isolated is substantially pure.

As used herein, “substantially pure” refers to material which is at least 50% pure (i.e., free from contaminants), at least 90% pure, at least 95% pure, at least 98% pure, or at least 99% pure.

The term “immunoconjugate” or “conjugate” as used herein refers to a compound or a derivative thereof that is linked to a cell binding agent (i.e., an anti-CD37 antibody or fragment thereof) and is defined by a generic formula: C-L-A, wherein C=cytotoxin, L=linker, and A=cell binding agent or anti-CD37 antibody or antibody fragment. Immunoconjugates can also be defined by the generic formula in reverse order: A-L-C.

A “linker” is any chemical moiety that is capable of linking a compound, usually a drug, such as a maytansinoid, to a cell-binding agent such as an anti CD37 antibody or a fragment thereof in a stable, covalent manner. Linkers can be susceptible to or be substantially resistant to acid-induced cleavage, light-induced cleavage, peptidase-induced cleavage, esterase induced cleavage, and disulfide bond cleavage, at conditions under which the compound or the antibody remains active. Suitable linkers are well known in the art and include, for example, disulfide groups, thioether groups, acid labile groups, photolabile groups, peptidase labile groups and esterase labile groups. Linkers also include charged linkers, and hydrophilic forms thereof as described herein and know in the art.

The terms “cancer” and “cancerous” refer to or describe the physiological condition in mammals in which a population of cells are characterized by unregulated cell growth. Examples of cancer include, but are not limited to, carcinoma, lymphoma, blastoma, sarcoma, and leukemia. More particular examples of “cancer” or “tumorigenic” diseases include B-cell lymphomas including NHL, precursor B-cell lymphoblastic leukemia/lymphoma and mature B-cell neoplasms, such as B-cell chronic lymphocytic leukemia (CLL)/small lymphocytic lymphoma (SLL), B-cell prolymphocytic leukemia, lymphoplasmacytic lymphoma, mantle cell lymphoma (MCL), follicular lymphoma (FL), including low-grade, intermediate-grade and high-grade FL, cutaneous follicle center lymphoma, marginal zone B-cell lymphoma (MALT type, nodal and splenic type), hairy cell leukemia, diffuse large B-cell lymphoma (DLBCL), Burkitt's lymphoma (BL), plasmacytoma, plasma cell myeloma, post-transplant lymphoproliferative disorder, and anaplastic large-cell lymphoma (ALCL).

“Tumor” and “neoplasm” refer to any mass that result from excessive cell growth or proliferation, either benign (noncancerous) or malignant (cancerous) including pre-cancerous lesions.

The terms “cancer cell,” “tumor cell,” and grammatical equivalents refer to the total population of cells derived from a tumor or a pre-cancerous lesion, including both non-tumorigenic cells, which comprise the bulk of the tumor cell population, and tumorigenic stem cells (cancer stem cells). As used herein, the term “tumor cell” will be modified by the term “non-tumorigenic” when referring solely to those tumor cells lacking the capacity to renew and differentiate to distinguish those tumor cells from cancer stem cells.

The term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject.

Administration “in combination with” one or more further therapeutic agents includes simultaneous (concurrent) and consecutive administration in any order.

The term “pharmaceutical formulation” refers to a preparation which is in such form as to permit the biological activity of the active ingredient to be effective, and which contains no additional components which are unacceptably toxic to a subject to which the formulation would be administered. Such formulation can be sterile.

An “effective amount” of an antibody as disclosed herein is an amount sufficient to carry out a specifically stated purpose. An “effective amount” can be determined empirically and in a routine manner, in relation to the stated purpose.

The term “therapeutically effective amount” refers to an amount of an antibody or other drug effective to “treat” a disease or disorder in a subject or mammal. In the case of cancer, the therapeutically effective amount of the drug can reduce the number of cancer cells; reduce the tumor size; inhibit (i.e., slow to some extent or stop) cancer cell infiltration into peripheral organs; inhibit (i.e., slow to some extent or stop) tumor metastasis; inhibit, to some extent, tumor growth; and/or relieve to some extent one or more of the symptoms associated with the cancer. See the definition herein of “treating.” To the extent the drug can prevent growth and/or kill existing cancer cells, it can be cytostatic and/or cytotoxic. A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

The term “respond favorably” generally refers to causing a beneficial state in a subject. With respect to cancer treatment, the term refers to providing a therapeutic effect on the subject. Positive therapeutic effects in cancer can be measured in a number of ways (See, W. A. Weber, J. Nucl. Med. 50:1S-10S (2009)). For example, tumor growth inhibition, molecular marker expression, serum marker expression, and molecular imaging techniques can all be used to assess therapeutic efficacy of an anti-cancer therapeutic. With respect to tumor growth inhibition, according to NCI standards, a T/C≦42% is the minimum level of anti-tumor activity. A T/C<10% is considered a high anti-tumor activity level, with T/C (%)=Median tumor volume of the treated/Median tumor volume of the control×100. Progression-free survival (PFS), disease-free survival (DFS), or overall survival (OS) can also be used to assess the therapeutic efficacy of an anti-cancer therapeutic.

PFS, DFS, and OS can be measured by standards set by the National Cancer Institute and the U.S. Food and Drug Administration for the approval of new drugs. See Johnson et al, (2003) J. Clin. Oncol. 21(7):1404-1411. “Progression free survival” (PFS), also referred to as or “Time to Tumor Progression” (YIP) indicates the length of time during and after treatment that the cancer does not grow. Progression-free survival includes the amount of time patients have experienced a complete response or a partial response, as well as the amount of time patients have experienced stable disease. “Disease free survival” (DFS) refers to the length of time during and after treatment that the patient remains free of disease. “Overall Survival” (OS) refers to a prolongation in life expectancy as compared to naive or untreated individuals or patients.

The, word “label” when used herein refers to a detectable compound or composition which is conjugated directly or indirectly to the antibody so as to generate a “labeled” antibody. The label can be detectable by itself (e.g., radioisotope labels or fluorescent labels) or, in the case of an enzymatic label, can catalyze chemical alteration of a substrate compound or composition which is detectable.

A “chemotherapeutic agent” is a chemical compound useful in the treatment of cancer, regardless of mechanism of action. Classes of chemotherapeutic agents include, but are not limited to alkylating agents, antimetabolites, spindle poison plant alkaloids, cytoxic/antitumor antibiotics, topoisomerase inhibitors, antibodies, photosensitizers, and kinase inhibitors. Chemotherapeutic agents include compounds used in “targeted therapy” and conventional chemotherapy.

Terms such as “treating” or “treatment” or “to treat” or “alleviating” or “to alleviate” refer to therapeutic measures that cure, slow down, lessen symptoms of and/or halt progression of a diagnosed pathologic condition or disorder, Thus, those in need of treatment include those already diagnosed with or suspected of having the disorder. Prophylactic or preventative measures refer to therapeutic measures that prevent and/or slow the development of a targeted pathologic condition or disorder. Thus, those in need of prophylactic or preventative measures include those prone to have the disorder and those in whom the disorder is to be prevented. In certain embodiments, a subject is successfully “treated” for cancer according to the methods of the present invention if the patient shows one or more of the following; a reduction in the number of or complete absence of cancer cells; a reduction in the tumor size; inhibition of or an absence of cancer cell infiltration into peripheral organs including, for example, the spread of cancer into soft tissue and bone; inhibition of or an absence of tumor metastasis; inhibition or an absence of tumor growth; relief of one or more symptoms associated with the specific cancer; reduced morbidity and mortality; improvement in quality of life; reduction in tumorigenicity, tumorigenic frequency, or tumorigenic capacity, of a tumor; reduction in the number or frequency of cancer stem cells in a tumor; differentiation of tumorigenic cells to a non-tumorigenic state; or some combination of effects.

As used in the present disclosure and claims, the singular forms “a,” “an,” and “the” include plural forms unless the context clearly dictates otherwise.

It is understood that wherever embodiments are described herein with the language “comprising,” otherwise analogous embodiments described in terms of “consisting of” and/or “consisting essentially of” are also provided.

The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### II. Biological Samples

Biological samples are often fixed with a fixative. Aldehyde fixatives such as formalin (formaldehyde) and glutaraldehyde are typically used Samples fixed using other fixation techniques such as alcohol immersion (Battifora and Kopinski, J. Histochem. Cytochem. (1986) 34:1095) are also suitable. The samples used may also be embedded in paraffin. In one embodiment, the samples are both formalin-fixed and paraffin-embedded (FFPE). In another embodiment, the FFPE block is hematoxylin and eosin stained prior to selecting one or more portions for analysis in order to select specific area(s) for the FFPE core sample. Methods of preparing tissue blocks from these particulate specimens have been used in previous IHC studies of various prognostic factors, and/or is well known to those of skill in the art (see, for example, Abbondanzo et al., Am J Clin Pathol. 1990 May; 93(5):698-702; Allred et al., Arch Surg. 1990 January; 125(1):107-13).

Briefly, any intact organ or tissue may be cut into fairly small pieces and incubated in various fixatives (e.g., formalin, alcohol, etc) for varying periods of time until the tissue is “fixed”. The samples may be virtually any intact tissue surgically removed from the body. The samples may be cut into reasonably small piece(s) that fit on the equipment routinely used in histopathology laboratories. The size of the cut pieces typically ranges from a few millimeters to a few centimeters. The biological sample can also be fluidic extracts, blood, plasma, serum, spinal fluid, bone marrow aspirate, bone marrow biopsy, lymph fluid, or splenic preparations.

### III. Detection Antibody Conjugates

The present invention further provides antibodies against CD37, generally of the monoclonal type, that are linked to at least one agent to form a detection antibody conjugate. In order to increase the efficacy of antibody molecules as diagnostic it is conventional to link or covalently bind or complex at least one desired molecule or moiety. Such a molecule or moiety may be, but is not limited to, at least one reporter molecule. A reporter molecule is defined as any moiety that may be detected using an assay. Non-limiting examples of reporter molecules that have been conjugated to antibodies include enzymes, radiolabels, haptens, fluorescent labels, phosphorescent molecules, chemiluminescent molecules, chromophores, luminescent molecules, photoaffinity molecules, colored particles and/or ligands, such as biotin.

Any antibody of sufficient selectivity, specificity or affinity may be employed as the basis for a detection antibody conjugate. Such properties may be evaluated using conventional immunological screening methodology known to those of skill in the art. Sites for binding to biological active molecules in the antibody molecule, in addition to the canonical antigen binding sites, include sites that reside in the variable, domain that, can bind the antigen. In addition, the variable domain is involved in antibody self-binding (Kang et al., 1988) and contains epitopes (idiotopes) recognized by anti-antibodies (Kohler et al., 1989).

Certain examples of antibody conjugates are those conjugates in which the antibody is linked to a detectable label. “Detectable labels” are compounds and/or elements that can be detected due to their specific functional properties, and/or chemical characteristics, the use of which allows the antibody to which they are attached to be detected, and/or further quantified if desired.

Many appropriate imaging agents are known in the art, as are methods for their attachment to antibodies (see, for e.g., U.S. Pat. Nos. 5,021,236; 4,938,948; and 4,472,509, each incorporated herein by reference). The imaging moieties used can be paramagnetic ions; radioactive isotopes; fluorochromes; NMR-detectable substances; and/or X-ray imaging, for example.

Exemplary fluorescent labels contemplated for use as conjugates include Alexa 350, Alexa 430, Alexa 488, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Dylight 488, Fluorescein Isothiocyanate (FITC), Green fluorescent protein (GFP), HEX, 6-JOE, Oregon Green 488, Oregon Green 500, Oregon Green 514, Pacific Blue, Phycoerythrin, REG, Rhodamine Green, Rhodamine Red, tetramethyl rhodamine (TMR), Renographin, ROX, TAMRA, TET, Tetramethylrhodamine, Texas Red, and derivatives of these labels (i.e., halogenated analogues, modified with isothiocynate or other linker for conjugating, etc.), for example.

Detection antibody conjugates contemplated in the present invention include those for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase and/or glucose oxidase. Preferred secondary binding ligands are biotin and/or avidin and streptavidin compounds. The use of such labels is well known to those of skill in the art and are described, for example, in U.S. Pat. Nos. 3,817,837; 3,850,752; 3,939,350; 3,996,345; 4,277,437; 4,275,149 and 4,366,241; each incorporated herein by reference.

Molecules containing azido groups may also be used to form covalent bonds to proteins through reactive nitrene intermediates that are generated by low intensity ultraviolet light (Potter & Haley, 1983). In particular, 2- and 8-azido analogues of purine nucleotides have been used as site-directed photoprobes to identify nucleotide binding proteins in crude cell extracts (Owens & Haley, 1987; Atherton et al., 1985). The 2- and 8-azido nucleotides have also been used to map nucleotide binding domains of purified proteins (Khatoon et al., 1989; King et al., 1989; and Dholakia et al., 1989) and may be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the antibody (U.S. Pat. Nos. 4,472,509 and 4,938,948, each incorporated herein by reference). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors, for example, is achieved using monoclonal antibodies, and the detectable imaging moieties are bound to the antibody using linkers such as methyl-p-hydroxybenzimidate or N-succinimidyl-3-(4-hydroxyphenyl)propionate.

In other embodiments, derivatization of immunoglobulins by selectively introducing sulfhydryl groups in the Fc region of an immunoglobulin using reaction conditions that do not alter the antibody combining site are contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region, have also been disclosed in the literature (O'Shannessy et al., 1987).

In other embodiments of the invention, immunoglobulins are radiolabeled, with nuclides such as tritium. In additional embodiments, nanogold particles (such as sizes from about 0.5 nm-40 nm) and/or Quantum Dots (Hayward, Calif.) are employed.

### IV. Enzymes and Substrates (Chromagens)

The use of substrates and indicators is contemplated for detection of CD37, such as the exemplary embodiments provided below, for example.

Horseradish peroxidase (HRP) is an enzyme that first forms a complex with hydrogen peroxide and then causes it to decompose, resulting in water and atomic oxygen. Like many other enzymes, HRP and some HRP-like activities can be inhibited by excess substrate. The complex formed between HRP and excess hydrogen peroxide is catalytically inactive and in the absence of an electron donor (e.g. chromogenic substance) is reversibly inhibited. It is the excess hydrogen peroxide and the absence of an electron donor that brings about, quenching of endogenous HRP activities.

When used in assays systems, HRP can also be used to convert a defined substrate into its activated chromagen, thus causing a color change. The HRP enzyme may be conjugated to an antibody, protein, peptide, polymer, or other molecule by a number of methods. Such methods are known in the art. Adding glutaraldehyde to a solution containing an admixture of HRP and antibody will result in more antibody molecules being conjugated to each other than to the enzyme. In the two-step procedure, HRP reacts with the bifunctional reagents first. In the second stage, only activated HRP is admixed with the antibody, resulting in much more efficient labelling and no polymerization. HRP is also conjugated to (strept)avidin using the two-step glutaraldehyde procedure. This form is used in procedures where LAB and LSAB are substrate, for example. Conjugation with biotin also involves two steps, as biotin must first be derivatized to the biotinyl-N-hydroxysuccinimide ester or to biotin, hydrazide before it, can be reacted with the epsilonamino groups of the HRP enzyme.

3,3′-diaminobenzidine (DAB) is a substrate for enzymes such as HRP that produces a brown end product that is highly insoluble in alcohol and other organic solvents. Oxidation of DAB also causes polymerization, resulting in the ability to react with osmium tetroxide, and thus increasing its staining intensity and electron density. Of the several metals and methods used to intensify the optical density of polymerized DAB, gold chloride in combination with silver sulfide appears to be the most successful.

3-Amino-9-ethylcarbazole (AEC), is a substrate for enzymes such as HRP, and upon oxidation, forms a rose-red end product that is alcohol soluble. Therefore, specimens processed with AEC must not be immersed in alcohol or alcoholic solutions (e.g., Harris' hematoxylin). Instead, an aqueous counterstain and mounting medium should be used AEC is unfortunately susceptible to further oxidation and, when exposed to excessive light, will fade in intensity. Storage in the dark is therefore recommended.

4-Chloro-1-naphthol (CN) is a substrate for enzymes such as HRP that precipitates as a blue end product. Because CN is soluble in alcohol and other organic solvents, the specimen must not be dehydrated, exposed to alcoholic counterstains, or coverslipped with mounting media containing organic solvents. Unlike DAB, CN tends to diffuse from the site of precipitation.

p-Phenylenediamine dihydrochloride/pyrocatechol (Hanker-Yates reagent) is a substrate for enzymes such as HRP that gives a blue-black reaction product that is insoluble in alcohol and other organic solvents. Like polymerized DAB, this reaction product can be osmicated. Varying results have been achieved with Hanker-Yates reagent in immunoperoxidase techniques.

Calf intestine alkaline phosphatase (AP) (molecular weight 100 kD) removes (by hydrolysis) and transfers phosphate groups from organic esters by breaking the P-0 bond; an intermediate enzyme-substrate bond is briefly formed. The chief metal activators for AP are Mg++, Mn++ and Ca++.

AP had not been used extensively in immunohistochemistry until publication of the e unlabeled alkaline phosphataseantialkaline phosphatase (APAAP) procedure. The soluble immune complexes utilized in this procedure have molecular weights of approximately 560 kD. The major advantage of the APAAP procedure compared to the PAP technique is the lack of interference posed by endogenous peroxidase activity. Because of the potential distraction of endogenous peroxidase activity on PAP staining, the APAAP technique is recommended for use on blood and bone marrow smears. Endogenous alkaline phosphatase activity from bone, kidney, liver and some white cells can be inhibited by the addition of 1 mM levamisole to the substrate solution, although 5 mM has been found to be more effective. Intestinal alkaline phosphatases are not adequately inhibited by levamisole.

In the immunoalkaline phosphatase staining method, the enzyme hydrolyzes naphthol phosphate esters (substrate) to phenolic compounds and phosphates. The phenols couple to colorless diazonium salts (chromogen) to produce insoluble, colored azo dyes. Several different combinations of substrates and chromogens have been used successfully.

Naphthol AS-MX phosphate AP substrate can be used in its acid form or as the sodium salt. The chromogen substrate Fast Red TR and Fast Blue BB produce a bright red or blue end product, respectively. Both are soluble in alcoholic and other organic solvents, so aqueous mounting media must be used Fast Red TR is preferred when staining cell smears.

Additional exemplary substrates include naphthol AS-BI phosphate, naphthol AS-TR phosphate and 5-bromo-4-chloro-3-indoxyl phosphate (BLIP). Other possible chromogens include Fast Red LB, Fast Garnet GBC, Nitro Blue Tetrazolium (NBT) and iodonitrotetrazolium Violet (INT), for example.

### V. Immunodetection Methods

In still further embodiments, the present invention concerns immunodetection methods for binding, purifying, removing, quantifying and/or otherwise generally detecting biological components such as CD37 as contemplated by the present invention. The antibodies prepared in accordance with the present invention may be employed to detect CD37. Some immunodetection methods include immunohistochemistry, flow cytometry, enzyme linked immunosorbent assay (ELISA), radioimmunoassay (RIA), immunoradiometric assay, fluoroimmunoassay, chemiluminescent assay, bioluminescent assay, and Western blot to mention a few. The steps of various useful immunodetection methods have been described in the scientific literature, such as, e.g., Doolittle M H and Ben-Zeev O, Methods Mol Biol. 1999; 109:215-37; Gulbis B and Galand P, Hum Pathol. 1993 December; 24(12):1271-85; and De Jager R et al., Semin Nucl Med. 1993 April; 23(2):165-79, each incorporated herein by reference.

In general, the immunobinding methods include obtaining a sample suspected of comprising ligand protein, polypeptide and/or peptide, and contacting the sample with a first anti-ligand antibody in accordance with the present invention, as the case may be, under conditions effective to allow the formation of immunocomplexes.

In terms of antigen detection, the biological sample analyzed may be any sample in which it is desirable to detect CD37, such as a fluidic extract, blood, plasma, serum, spinal fluid, lymph fluid, tissue section or specimen, homogenized tissue extract, biopsy aspirates, a cell, separated and/or purified forms of CD37-containing compositions, or any biological fluid. In some embodiments, blood, plasma, tissue or lymph samples or extracts are used.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any ligand protein antigens present. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. U.S. patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art.

The anti-ligand antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding agent that has binding affinity for the antibody. In these cases, the second binding agent may be linked to a detectable label. The second binding agent is itself often an antibody, which may thus be termed a “secondary” antibody. The primary immune complexes are contacted with the labeled, secondary binding agent, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two-step approach. A second binding agent, such as an antibody, that has binding affinity for the antibody is used to form secondary immune complexes, as described above. After washing, the secondary immune complexes are contacted with a third binding agent or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

In another embodiment, a biotinylated monoclonal or polyclonal antibody is used to detect the target antigen(s), and a second step antibody is then used to detect the biotin attached to the complexed biotin. In that method the sample to be tested is first incubated in a solution comprising the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution comprising the second step antibody against biotin. This second step antibody is labeled, as for example with an enzyme that can be used to detect the presence of the antibody/antigen complex by histoenzymology using a chromogen substrate. With suitable amplification, a conjugate can be produced that is macroscopically visible.

In one embodiment, immunohistochemistry (IHC) is used for immunological detection. Using IHC, detection of CD37 in a sample can be achieved by targeting a sample with a probe, e.g., an anti-CD37 antibody. The probe can be linked, either directly or indirectly to a detectable label or can be detected by another probe that is linked, either directly or indirectly to a detectable label.

In some embodiments, the IHC can distinguish between different levels of protein expression, e.g., calibrated IHC. In some embodiments, the IHC can distinguish staining intensity for samples having low cell surface CD37 expression, intermediate CD37 cell surface expression, or high CD37 cell surface expression.

In some embodiments, the IHC can distinguish between staining intensity and staining uniformity. In one embodiment, immunological detection (by immunohistochemistry) of CD37 is scored for both intensity and uniformity (percent of stained cells—membrane only). Comparative scales for CD37 expression for intensity correlate as 0—Negative, 0-1—Very Weak, 1—Weak, 1-2—Weak to Moderate, 2—Moderate, 2-3—Moderate to Strong, 3—Strong, 3+—Very Strong. Quantitatively, Score 0 represents that no staining is observed or membrane staining is observed in less than 10% of tumor cells. Score 1 represents that a faint/barely perceptible membrane staining is detected in more than 10% of the tumor cells. The cells are only stained in part of their membrane. For Score 2, a moderate complete membrane staining is observed in more than 10% of the tumor cells. Lastly, Score 3 represents that a strong complete membrane staining is observed in more than 10% of the tumor cells. Those samples with 0 or 1 score for CD37 expression can be characterized as not overexpressing CD37, whereas those samples with 2 or 3 scores can be characterized as overexpressing CD37. Samples overexpressing CD37 can also be rated by immunohistochemical scores corresponding to the number of copies of CD37 molecules expressed per cell, or antibodies bound per cell (ABC), and can been determined biochemically. Comparative scales for CD37 percent cell membrane staining uniformity correlate as follows: 0—Negative, Focal—<25%, heterogeneous (hetero)—25-75%, and homogeneous (homo)—>75%.

IHC can be performed manually or using an automated system (e.g., using an automated stainer). Thus, IHC can be performed on cells, cell pellets, tissues, preparations from blood, plasma, serum, or lymph fluid, etc. In some embodiments, the samples are fixed samples. In some embodiments, the samples are paraffin embedded samples. In some embodiments, the samples are formalin fixed and paraffin embedded samples.

Another known method of immunodetection takes advantage of the immuno-PCR (Polymerase Chain Reaction) methodology. The PCR method uses a DNA/biotin/streptavidin/antibody complex that is washed out with a low pH or high salt buffer that releases the antibody. The resulting wash solution is then used to carry out a PCR reaction with suitable primers with appropriate controls. In specific embodiments, the enormous amplification capability and specificity of PCR can be utilized to detect a single antigen molecule. Such detection may take place in real-time. For example, the use of quantitative real-time PCR is contemplated.

In one embodiment, flow cytometry is used for immunological detection. Thus, for example, the number of antibodies bound per cell (ABC) can be assessed using flow cytometry. A high number of anti-CD37 antibodies bound per cell can indicate high CD37 expression levels and a high likelihood to be susceptible to treatment with an anti-CD37 antibody or immunoconjugate thereof.

### VI. Nucleic Acid Hybridization

In situ hybridization is generally carried out on cells or tissue sections fixed to slides. In situ hybridization may be performed by several conventional methodologies (See for e.g., Leitch et al. In situ Hybridization: a practical guide, Oxford BIOS Scientific Publishers, Microscopy handbooks v. 27 (1994)). In one in situ procedure, fluorescent dyes (such as fluorescein isothiocyanate (FITC) that fluoresces green when excited by an Argon ion laser) are used to label a nucleic acid sequence probe that is complementary to a target nucleotide sequence in the cell. Each cell comprising the target nucleotide sequence will bind the labeled probe, producing a fluorescent signal upon exposure of the cells to a light source of a wavelength appropriate for excitation of the specific fluorochrome used.

Various degrees of hybridization stringency can be employed. As the hybridization conditions become more stringent, a greater degree of complementarity is required between the probe and target to form and maintain a stable, duplex. Stringency is increased by raising temperature, lowering salt concentration, or raising formamide concentration. Adding dextran sulfate or raising its concentration may also increase the effective concentration of labeled probe to increase the rate of hybridization and ultimate signal intensity. After hybridization, slides are washed in a solution generally comprising reagents similar to those found in the hybridization solution with washing time varying from minutes to hours depending on required stringency. Longer or more stringent washes typically lower nonspecific background but run the risk of decreasing overall sensitivity.

Probes used in nucleic hybridization analysis may be either RNA or DNA oligonucleotides or polynucleotides and may contain not only naturally-occurring nucleotides but their analogs, like digoxygenin dCTP, biotin dcTP 7-azaguanosine, azidothymidine, inosine, or uridine, for example. Other useful probes include peptide probes and analogues thereof, branched gene DNA, peptidometics, peptide nucleic acid (PNA) and/or antibodies, for example.

Probes should have sufficient complementarity to the target nucleic acid sequence of interest so that stable and specific binding occurs between the target nucleic acid sequence and the probe. The degree of homology required for stable hybridization varies with the stringency of the hybridization medium and/or wash medium. Preferably, completely homologous probes are employed in the present invention, but persons of skill in the art will readily appreciate that probes exhibiting lesser but sufficient homology can be used in the present invention (see for e.g., Sambrook, J., Fritsch, E. F., Maniatis, T., Molecular Cloning A Laboratory Manual, Cold Spring Harbor Press, (1989)).

Probes may also be generated and chosen by several means including, but not limited to, mapping by in situ hybridization, somatic cell hybrid panels, or spot blots of sorted chromosomes; chromosomal linkage analysis; or cloned and isolated from sorted chromosome libraries from human cell lines or somatic cell hybrids with human chromosomes, radiation somatic cell hybrids, microdissection of a chromosome region, or from yeast artificial chromosomes (YACs) identified by PCR primers specific for a unique chromosome locus or other suitable means like an adjacent YAC clone. Probes may be genomic DNA, cDNA, or RNA cloned in a plasmid, phage, cosmid, YAC, Bacterial Artificial Chromosomes (BACs), viral vector, or any other suitable vector. Probes may be cloned or synthesized chemically by conventional methods. When cloned, the isolated probe nucleic acid fragments are typically inserted into a vector, such as lambda phage, pBR322, M13, or vectors containing the SP6 or T7 promoter and cloned as a library in a bacterial host. [See for e.g., Sambrook, J., Fritsch, E. F., Maniatis, T., Molecular Cloning A Laboratory Manual, Cold Spring Harbor Press, (1989)].

Probes are preferably labeled, such as with a fluorophore, for example. Examples of fluorophores include, but are not limited to, rare earth chelates (europium chelates), Texas Red, rhodamine, fluorescein, dansyl, Lissamine, umbelliferone, phycocrytherin, phycocyanin, or commercially available fluorophors such SPECTRUM ORANGE™ and SPECTRUM GREEN™ and/or derivatives of any one or more of the above. Multiple probes used in the assay may be labeled with more than one distinguishable fluorescent or pigment color. These color differences provide a means to identify the hybridization positions of specific probes. Moreover, probes that are not separated spatially can be identified by a different color light or pigment resulting from mixing two other colors (e.g., light red+green=yellow) pigment (e.g., blue+yellow=green) or by using a filter set that passes only one color at a time.

Probes can be labeled directly or indirectly with the fluorophor, utilizing conventional methodology known to one with skill in the art.

### VII. Detection Kits and Compositions

Also provided by the invention are kits for use in the practice of the present invention as disclosed herein. Such kits may comprise containers, each with one or more of the various reagents (typically in concentrated form) utilized in the methods, including, for example, one or more binding agents (antibodies), already attached to a marker or optionally with reagents for coupling a binding agent to an antibody or nucleic acid molecule (as well as the marker itself); buffers, the appropriate nucleotide triphosphates (e.g., dATP, dCTP, dGTP, dTTP, dUTP, ATP, CTP, GTP and UTP), reverse transcriptase, DNA polymerase, RNA polymerase, and one or more sequence-specific or degenerate primers for use in detection of nucleic acid molecules by amplification; and/or reagents and instrumentation for the isolation (optionally by microdissection) to support the practice of the invention. A label or indicator describing, or a set of instructions for use of, kit components in a ligand detection method of the present invention, will also be typically included, where the instructions may be associated with a package insert and/or the packaging of the kit or the components thereof.

In still further embodiments, the present invention concerns immunodetection kits for use with the immunodetection methods described above. As the antibodies are generally used to detect CD37, the antibodies will preferably be included in the kit. The immunodetection kits will thus comprise, in suitable container means, a first antibody that binds to CD37, and/or optionally, an immunodetection reagent and/or further optionally, a CD37 protein or cell or sample containing a CD37 protein.

The immunodetection reagents of the kit may take any one of a variety of forms, including those detectable labels that are associated and/or linked to the given antibody. Detectable labels that are associated with and/or attached to a secondary binding ligand are also contemplated. Exemplary secondary ligands are those secondary antibodies that have binding affinity for the first antibody.

Further suitable immunodetection reagents for use in the present kits include the two-component reagent that comprises a secondary antibody that has binding affinity for the first antibody, along with a third antibody that has binding affinity for the second antibody, the third antibody being linked to a detectable label. As noted above, a number of exemplary labels are known in the art and/or all such labels may be suitably employed in connection with the present invention.

The kits may further comprise one or more therapeutic agents for the treatment of cancer, such as an anti-CD37 immunoconjugate and/or a chemotherapeutic agent.

The kit may further comprise an a CD37 detection reagent used to measure CD37 expression in a subject comprising a CD37 detection reagent, and instructions for use. In one embodiment, the CD37 detection reagent comprises a CD37 binding peptide, protein or a molecular probe (i.e., nucleic acid). In another embodiment, the CD37 detection reagent is an anti-CD37 antibody. In another embodiment, the kit further comprises a secondary antibody which binds the anti-CD37 antibody. In one embodiment the CD37-specific antibody is included at a concentration of 2.1, 4.2, and 8.4 μg/mL. In another embodiment, the antibody is included in concentrated solution with instructions for dilutions to achieve a final concentration of 2.1, 4.2, and 8.4 μg/mL. In another embodiment, the kit further comprises a detection reagent selected from the group consisting of: an enzyme, a fluorophore, a radioactive label, and a luminophore. In another embodiment, the detection reagent is selected from the group consisting of: biotin, digoxigenin, fluorescein, tritium, and rhodamine.

The kit can also include instructions for detection and scoring of CD37 expression. The kit can also include control or reference samples. Non-limiting examples of control or reference samples include cell pellets or tissue culture cell lines derived from negative normal tissue (negative control) or tumor (positive control) samples. Exemplary positive control cell lines include Daudi, Ramos. Namahva and a negative control includes Colo205 and cell lines stably or transiently transfected with an expression vector that expresses CD37.

### VIII. CD37-Binding Agents

Any antibodies that bind CD37 can be used in the detection methods of the present invention. Examples of therapeutically effective anti-CD37 antibodies, which can be used in the present methods can be found in US Published Appl. No. 2011/0256153, which is herein incorporated by reference. For example, the anti-CD37 antibody can be mouse, chimierc, or humanized CD37-3, CD37-12, CD37-38, CD37-50, CD37-51, CD37-56 or CD37-57. The full-length amino acid sequence for CD37 is known in the art and also provided herein as represented by SEQ ID NO: 1. A specifically useful antibody for detection of CD37 is the mouse monoclonal anti-huCD37 clone CT1 (Leica # NCL-CD37). An example of a therapeutically effective anti-CD37 antibody is huCD37-3-SMCC-DM1. The polypeptide of SEQ ID NO: 57 corresponds to the variable domain heavy chain version 1.0 of huCD37-3. The polypeptide of SEQ ID NO 58 corresponds to the variable domain heavy chain version 1.1 of huCD37-3, and the polypeptide of SEQ ID NO: 74 corresponds to the variable domain light chain of huCD37-3, respectively. In certain embodiments, the CD37 antibody can comprise a light chain encoded by the recombinant plasmid DNA phuCD37-3LC (ATCC Deposit Designation PTA-10722, deposited with the ATCC (10801 University Boulevard, Manassas, Va. 20110) on Mar. 18, 2010). In certain embodiments, the CD37 antibody can comprise a heavy chain encoded by the recombinant plasmid. DNA phuCD37-3HCv.1.0 (ATCC Deposit Designation PTA-10723, deposited with the ATCC on Mar. 18, 2010). In certain embodiments, the CD37 antibody can comprise a light chain encoded by the recombinant plasmid DNA phuCD37-3LC (PTA-10722) and a heavy chain encoded by the recombinant plasmid DNA phuCD37-3HCv.1.0 (PTA-10723). In certain embodiments, the CD37 antibody can comprise the VL-CDRs encoded by the recombinant plasmid DNA phuCD37-3LC (PTA-1072.2) and the VH-CDRs encoded by the recombinant plasmid DNA phuCD37-3HCv.1.0 (PTA-10723).

Examples of CD37 immunoconjugates useful in the therapeutic methods of the invention are provided below.

### IX. CD37 Immunoconjugates

The present invention is also increasing the efficacy of conjugates (also referred to herein as immunoconjugates), comprising the anti-CD37 antibodies, antibody fragments, functional equivalents, improved antibodies and their aspects as disclosed herein, linked or conjugated to a cytotoxin (drug) or prodrug. Exemplary CD37 antibodies and immunoconjugates can be found in U.S. Published Application No 2011/0256153, which is herein incorporated by reference. A particularly effective therapeutic immunoconjugate of the invention comprises the huCD37-3 antibody described above.

Suitable drugs or prodrugs are known in the art. In certain embodiments, drugs or prodrugs are cytotoxic agents. The cytotoxic agent used in the cytotoxic conjugate of the present invention can be any compound that results in the death of a cell, or induces cell death, or in some manner decreases cell viability, and includes, for example, maytansinoids and maytansinoid analogs, benzodiazepines, taxoids, CC-1065 and CC-1065 analogs, duocarmycins and duocarmycin analogs, enediynes, such as calicheamicins, dolastatin and dolastatin analogs including auristatins, tomaymycin derivatives, leptomycin derivatives, methotrexate, cisplatin, carboplatin, daunorubicin, doxorubicin, vincristine, vinblastine, melphalan, mitomycin C, chlorambucil and morpholino doxorubicin. In certain embodiments, the cytotoxic agents are maytansinoids and maytansinoids analogs.

The drug or prodrug can, for example, be linked to the anti-CD37 antibody, such as huCD37-3, or fragment thereof through a disulfide bond. The linker molecule or crosslinking agent comprises a reactive chemical group that can react with the anti-CD37 antibody or fragment thereof. In certain embodiments, reactive chemical groups for reaction with the cell-binding agent are N-succinimidyl esters and N-sulfosuccinimidyl esters. Additionally the linker molecule comprises a reactive chemical group, in certain embodiments a dithiopyridyl group that can react with the drug to form a disulfide bond. In certain embodiments, linker molecules include, for example, N-succinimidyl 3-(2-pyridyldithio) propionate (SPDP) (see, e.g., Carlsson et al., Biochem. J., 173: 723-737 (1978)), N-succinimidyl 4-(2-pyridyldithio)butanoate (SPDB) (see, e.g., U.S. Pat. No. 4,563,304), N-succinimidyl 4-(2-pyridyldithio)2-sulfobutanoate (sulfo-SPDB) (see US Publication No. 20090274713), N-succinimidyl 4-(2-pyridyldithio) pentanoate (SPP) (see, e.g., CAS Registry number 341498-08-6), 2-iminothiolane, or acetylsuccinic anhydride.

Antibody-maytansinoid conjugates with non-cleavable links can also be prepared. Such crosslinkers are described in the art (see ThermoScientific Pierce Crosslinking Technical Handbook and US Patent Application Publication No. 2005/0169933) and include but are not limited to, N-succinimidyl 4-(maleimidomethyl) cyclohexanecarboxylate (SMCC), N-succinimidyl-4-(N-maleimidomethyl)-cyclohexane-1-carboxy-(6-amidocaproate), which is a “long chain” analog of SMCC (LC-SMCC), κ-maleimidoundecanoic acid N-succinimidyl ester (KMUA), β-maleimidopropanoic acid N-succinimidyl ester (BMPS), γ-maleimidobutyric acid N-succinimidyl ester (GMBS), ε-maleimidocaproic acid N-hydroxysuccinimide ester (EMCS), m-maleimidobenzoyl-N-hydroxysuccinimide ester (MBS), N-(α-maleimidoacetoxy)-succinimide ester (AMAS), succinimidyl-6-(β-maleimidopropionamido)hexanoate (SMPH), N-succinimidyl 4-(p-maleimidophenyl)-butyrate (SMPB), and N-(p-maleimidophenyl)isocyanate (PMPI), N-succinimidyl-4-(iodoacetyl)-aminobenzoate (SIAB), N-succinimidyl iodoacetate (SIA), N-succinimidyl bromoacetate (SBA), and N-succinimidyl 3-(bromoacetamido)propionate (SBAP). In certain embodiments, the antibody is modified with crosslinking reagents such as succinimidyl 4-(N-maleimidomethyl)-cyclohexane-1-carboxylate (SMCC), sulfo-SMCC, maleimidobenzoyl-N-hydroxysuccinimide ester (MBS), sulfo-MBS or succinimidyl-iodoacetate, as described in the literature, to introduce 1-10 reactive groups (Yoshitake et al, Eur. J. Biochem., 101:395-399 (1979); Hashida et al, J. Applied Biochem., 56-63 (1984); and Liu et al, Biochem., 18:690-697 (1979)).

The present invention includes aspects wherein the average molar ratio of the cytotoxic agent (e.g., maytansinoid) to the cell-binding agent in the cell-binding agent cytotoxic agent conjugate is about 1 to about 10. The terms “MAR,” “Maytansinoid-Ab Ratio,” “drug load,” “DAR,” and “Drug-Ab Ratio” can be used herein to characterize the ratio of cytotoxic agent to cell-binding agent in a conjugate comprising a maytansinoid compound as the cytotoxic agent and an antibody or fragment thereof as the cell binding agent. Thus, in some embodiments, the MAR is about 1 to about 10, about 2 to about 7, about 3 to about 5, about 2.5 to about 4.5 (e.g., about 2.5, about 2.6, about 2.7, about 2.8, about 2.9, about 3.0, about 3.1, about 3.3, about 3.4, about 3.5, about 3.6, about 3.7, about 3.8, about 3.9, about 4.0, about 4.1, about 4.2, about 4.3, about 4.4, about 4.5), about 3.0 to about 4.0, about 3.2 to about 4.2, about 4.5 to 5.5 (e.g., about 4.5, about 4.6, about 4.7, about 4.8, about 4.9, about 5.0, about 5.1, about 5.2, about 5.3, about 5.4, about 5.5). In one aspect, the number of drug molecules that can be attached to a cell binding agent can average from about 2 to about 8 (e.g., 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1). In certain embodiments, the drug is N^{2′}-deacetyl-N^{2′}-(3-mercapto-1-oxopropyl)-maytansine (DM1) or N^{2′}-deacetyl-N^{2′}-(4-mercapto-4-methyl-1-oxopentyl) maytansine (DM4). Thus, in a certain embodiment, the antibody huCD37-3 is conjugated to DM1 or DM4.

### X. Correlation of CD37 Expression and Therapeutic Efficacy

In certain embodiments, the invention provides a method for identifying subjects with an increased likelihood for responding to CD37-targeting anti-cancer therapies. The invention is based, in part, on the discovery that elevated CD37 expression levels correlates with efficacy of CD37-targeting anti-cancer therapeutics and the discovery of methods of detecting a dynamic range of CD37 expression in B-cell samples.

Evaluation of patient samples and correlation to in vivo efficacy using xenograft models demonstrates the power of the expression analysis for selecting subjects more likely to respond to treatment. IHC provides a score for CD37 expression on tumor cells: 0 (no expression) to 3+ (very high levels of expression). Samples scoring 1, 2, 3, or 3+ for CD37 expression (or 2, 3, or 3+) have an increased, likelihood to respond to CD37-targeted anti-cancer therapies at clinically-relevant doses of CD37 immunoconjugates (e.g., 0.1 to 10 or more mg/kg xenograft dose of a CD37 immunoconjugate can approximate a 3.0 to 400 mg/m² in patients). Thus, identification of individuals having an elevated CD37 score would help identify those individuals who might respond to a clinically relevant dosage. As described in more detail below, sensitivity to CD37 therapeutics may correlate with CD37 scoring of 2 or higher, especially with level 3 scoring. Moreover, expression of more uniform levels of CD37 can also be used as an indicator of correlation of CD37 expression with therapeutic benefit. Thus, a homogenous staining uniformity or a combination of increased intensity with heterogenous staining uniformity could indicate increased CD37 expression. For example, scores of greater than 2 hetero may be used as a patient selection criterion for treatment with a CD37 therapeutic agent.

CD37 expression analysis also identifies patients in whom decreased levels of a CD37-targeting anti-cancer therapy (“low dose therapy”) can be effective to cause anti-tumor responses. As is appreciated in the art, compounds are generally administered at the smallest dosage that achieves the desired therapeutic response. This is specifically important for therapeutics that cause clinical, and often undesired, side effects. The ability to recognize those subjects with elevated CD37 expression levels allows for minimization of the dosage of the CD37-targeting therapeutic, thus decreasing possible side effects, while maintaining therapeutic efficacy.

### XI. Pharmaceutical Compositions and Therapeutic Methods

CD37-binding agents (including antibodies, immunoconjugates, and polypeptides) are useful in a variety of applications including, but not limited to, therapeutic treatment methods, such as the treatment of cancer. In certain embodiments, the agents are useful for inhibiting tumor growth, inducing differentiation, reducing tumor volume, and/or reducing the tumorigenicity of a tumor. The methods of use may be in vitro, ex vivo, or in vivo methods. In certain embodiments, the CD37-binding agent or antibody or immunoconjugate, or polypeptide is an antagonist of the human CD37 to which it binds.

In certain embodiments, the disease treated with the CD37-binding agent or antagonist (e.g., a huCD37-3 antibody or immunoconjugate) is a cancer. In certain embodiments, the cancer is characterized by tumors expressing CD37 to which the CD37-binding agent (e.g., antibody) binds.

The present invention provides for methods of treating cancer comprising administering a therapeutically effective amount of a CD37-binding agent to a subject (e.g., a subject in need of treatment). In certain embodiments, the cancer is selected from the group consisting of B cell lymphomas, NHL, precursor B cell lymphoblastic leukemia/lymphoma and mature B cell neoplasms, B cell chronic lymphocytic leukemia (CLL)/small lymphocytic lymphoma (SLL), B cell prolymphocytic leukemia, lymphoplasmacytic lymphoma, mantle cell lymphoma (MCL), follicular lymphoma (FL), low grade, intermediate-grade and high-grade (FL), cutaneous follicle center lymphoma, marginal zone B cell lymphoma, MALT type marginal zone B cell lymphoma, nodal marginal zone B cell lymphoma, splenic type marginal zone B cell lymphoma, hairy cell leukemia, diffuse large B cell lymphoma (DLBCL), Burkitt's lymphoma (BL), plasmacytoma, plasma cell myeloma, post-transplant lymphoproliferative disorder, Waldenstrom's macroglobulinemia, and anaplastic large-cell lymphoma (ALCL). In certain embodiments, the subject is a human.

The present invention further provides methods for inhibiting tumor growth using the antibodies or other agents described herein. In certain embodiments, the method of inhibiting the tumor growth comprises contacting the cell with a CD37-binding agent (e.g., antibody) in vitro. For example, an immortalized cell line or a cancer cell line that expresses CD37 is cultured in medium to which is added the antibody or other agent to inhibit tumor growth. In some embodiments, tumor cells are isolated from a patient sample such as, for example, a tissue biopsy, pleural effusion, or blood sample and cultured in medium to which is added an CD37-binding agent to inhibit tumor growth.

In some embodiments, the method of inhibiting tumor growth comprises contacting the tumor or tumor cells with the CD37-binding agent (e.g., antibody) in vivo. In certain embodiments, contacting a tumor or tumor cell with a CD37-binding agent is undertaken in an animal model. For example, CD37-binding agents can be administered to xenografts expressing one or more CD37s that have been grown in immunocompromised mice (e.g., NOD/SCID mice) to inhibit tumor growth. In some embodiments, the CD37-binding agent is administered at the same time or shortly after introduction of tumorigenic cells into the animal to prevent tumor growth. In some embodiments, the CD37-binding agent is administered as a therapeutic after the tumorigenic cells have grown to a specified size.

In certain embodiments, the method of inhibiting tumor growth comprises administering to a subject a therapeutically effective amount of a CD37-binding agent. In certain embodiments, the subject is a human. In certain embodiments, the subject has a tumor or has had a tumor removed.

Thus, in certain embodiments the inventions provides methods of treating cancer using a CD37-3 antibody (e.g., chimeric, humanized, or fully human) or immunoconjugate thereof, wherein the cancer is identified, using the methods described herein, as having increased CD37 expression. In a certain embodiment, the CD37-3 immunoconjugate is huCD37-3-SMCC-DM1.

In certain embodiments, formulations are prepared for storage and use by combining a purified antibody or agent of the present invention with a pharmaceutically acceptable vehicle (e.g., carrier, excipient) (Remington, The Science and Practice of Pharmacy 20th Edition Mack Publishing, 2000). Suitable pharmaceutically acceptable vehicles include, but are not limited to, nontoxic buffers such as phosphate, citrate, and other organic acids; salts such as sodium chloride; antioxidants including ascorbic acid and methionine; preservatives (e.g., octadecyldimethylbenzyl ammonium chloride; hexamethonium chloride; benzalkonium chloride; benzethonium chloride; phenol, butyl or benzyl alcohol; alkyl parabens, such as methyl or propyl paraben; catechol; resorcinol; cyclohexanol; 3-pentanol; and m-cresol); low molecular weight polypeptides (e.g., less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosaccharides, disaccharides, glucose, mannose, or dextrins; chelating agents such as EDTA; sugars such as sucrose, mannitol, trehalose or sorbitol; salt-forming counter-ions such as sodium; metal complexes (e.g., Zn-protein complexes); and non-ionic surfactants such as TWEEN or polyethylene glycol (PEG).

The pharmaceutical compositions of the present invention can be administered in any number of ways for either local or systemic treatment. Administration can be topical (such as to mucous membranes including vaginal and rectal delivery) such as transdermal patches, ointments, lotions, creams, gels, drops, suppositories, sprays, liquids and powders; pulmonary (e.g., by inhalation or insufflation of powders or aerosols, including by nebulizer; intratracheal, intranasal, epidermal and transdermal); oral; or parenteral including intravenous, intraarterial, subcutaneous, intraperitoneal or intramuscular injection or infusion; or intracranial (e.g., intrathecal or intraventricular) administration.

An antibody or immunoconjugate of the invention can be combined in a pharmaceutical combination formulation, or dosing regimen as combination therapy, with a second compound having anti-cancer properties. The second compound of the pharmaceutical combination formulation or dosing regimen preferably has complementary activities to the ADC of the combination such that they do not adversely affect each other. Pharmaceutical compositions comprising the CD37-binding agent and the second anti-cancer agent are also provided.

For the treatment of the disease, the appropriate dosage of an antibody or agent of the present invention depends on the type of disease to be treated, the severity and course of the disease, the responsiveness of the disease, whether the antibody or agent is administered for therapeutic or preventative purposes, previous therapy, patient's clinical history, and so on all at the discretion of the treating physician. The antibody or agent can be administered one time or over a series of treatments lasting from several days to several months, or until a cure is effected or a diminution of the disease state is achieved (e.g., reduction in tumor size). Optimal dosing schedules can be calculated from measurements of drug accumulation in the body of the patient and will vary depending on the relative potency of an individual antibody or agent. The administering physician can easily determine optimum dosages, dosing methodologies and repetition rates. In certain embodiments, dosage is from 0.01 μg to 100 mg per kg of body weight, and can be given once or more daily, weekly, monthly or yearly. In certain embodiments, the antibody or other CD37-binding agent is given weekly, on every two weeks or once every three weeks. In certain embodiments, the dosage of the antibody or other CD37-binding agent is from about 0.1 mg to about 20 mg per kg of body weight. The treating physician can estimate repetition rates for dosing based on measured residence times and concentrations of the drag in bodily fluids or tissues.

The combination therapy can provide “synergy” and prove “synergistic,” i.e., the effect achieved when the active ingredients used together is greater than the sum of the effects that results from using the compounds separately. A synergistic effect can be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect can be attained when the compounds are administered or delivered sequentially, e.g., by different injections in separate syringes. In general, during alternation therapy, an effective dosage of each active ingredient is administered sequentially, i.e., serially, whereas in combination therapy, effective dosages of two or more active ingredients are administered together.

Embodiments of the present disclosure can be further defined by reference to the following non-limiting examples, which describe in detail preparation of certain antibodies of the present disclosure and methods for using antibodies of the present disclosure. It will be apparent to those skilled in the art that many modifications, both to materials and methods, can be practiced without departing from the scope of the present disclosure.

### EXAMPLES

It is understood that the examples and embodiments described herein are for illustrative purposes only and that various modifications or changes in light thereof will be suggested to persons skilled in the art and are to be included within the spirit and purview of this application.

### Example 1

### Immunohistochemical Staining of CD37 in Cell Samples-Manual Methods

Formalin fixed paraffin embedded cell pellets and tissues were used as test samples with the following staining reagents and conditions.

Formalin-fixed paraffin-embedded (FFPE) patient lymphoma biopsies were stained with the murine anti-CD37 antibody clone CT1, (muIgG1, Leica, Cat# NCL-CD37), murine anti-CD20 antibody clone L26 (muIgG2a, Dako Cytomation, Cat# M 07 55), and the respective isotype control muIgG1 and muIgG2a antibodies from Coulter as follows. Slides containing samples were baked at 60° C. for 30 min in a drying oven and were deparaffinized and rehydrated by sequential immersion in the following solvents: xylenes, absolute ETOH, 95% ETOH and water. Following antigen retrieval in pH 9.5 Borg buffer (Biocare Medical) in a decloaker, slides were washed with PBS (Gibco), and sections were blocked in PBS containing 2% normal horse serum (Vector Labs) and avidin-biotin block (4 drops/mL, Vector Labs) for 30 min. To prepare working solutions of primary antibodies, the CD37-test and -isotype control articles were each diluted to 4.0 μg/mL in diluent [PBS containing 2% normal horse serum and biotin (4 drops/mL)]. The CD20-test and -isotype control articles were each diluted to 0.5 μg/mL in diluent. Slides were washed in PBS, and incubated at room temperature for 60 minutes with the test articles (anti-CD37 or anti-CD20), or control articles (muIgG1 or muIgG2a), followed by a 30 minute incubation with 10 μg/mL of a biotinylated horse anti-mouse IgG (H+L) secondary antibody (Vector Labs). Slides were washed again in PBS and incubated for 40 minutes with avidin-biotin-peroxidase complex (Vector Labs) to detect bound secondary antibody. Incubation for 5 minutes with DAB (3,3-diaminobenzidine tetrahydrochloride, Dako Cytomation) resulted in the color signal. Slides containing developed tissue sections were counterstained by immersion in a container filled with Hematoxylin (Biocare Medical) for 4 min. Slides were cleared of excess stain followed by a quick immersion in Lithium Carbonate solution (0.135 M Li₂CO₃ aqueous solution, Sigma Aldrich) which enhances the blue nuclear staining. Slides were dehydrated through two rinses each of 95% and 100% ETOH, followed by four washes of Xylene for 1 min each. Coverslips were mounted onto the slides using Mounting medium (Richard Allan Scientific).

FFPE samples were derived from tumor micro arrays, as well as human tissue blocks from three different CLL tumors, as outlined in Table 1.

TABLE 1
FFPE Test Samples

| FFPE Human Tissue | | |
|---|---|---|
| Samples | Description | Source |
| Tissue Micro array | 39 Cases | Biomax Cat# LYM401 |
| | 48 Cases | Biomax Cat# LM482 |
| CLL | 3 FFPE blocks | Analytical Biological |
| | | Services |

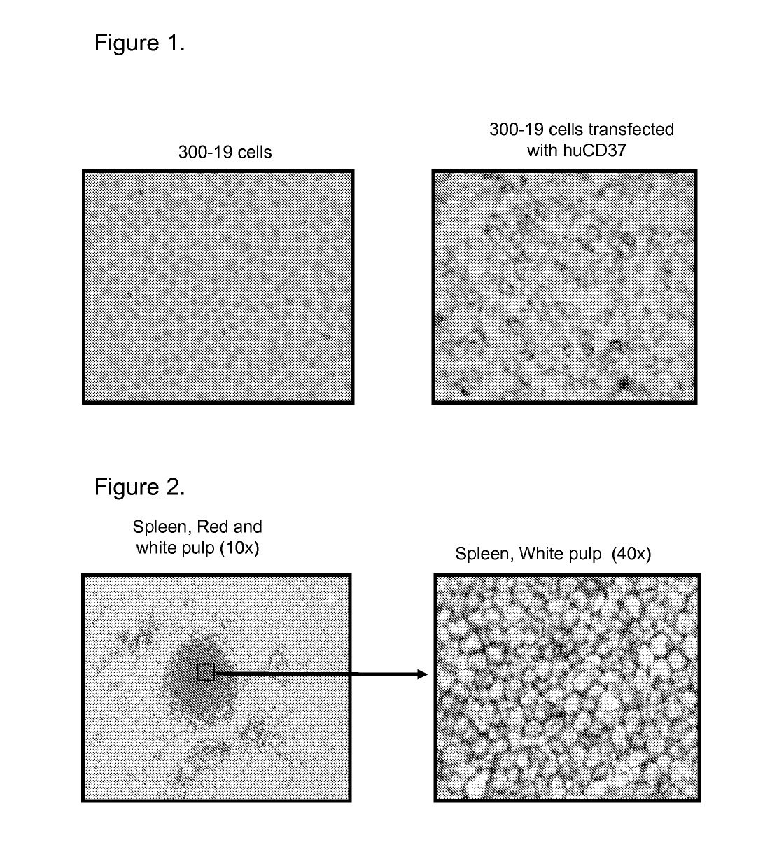

The CD37 test article, murine anti-CD37 antibody clone CT1, was tested to determine binding specificity to the huCD37 antigen. Using the reported IHC staining methods, FFPE sections of 300-19 and 300-19 transfected with huCD37 (300-19/huCD37) cell pellets were stained and evaluated for CD37. The CD37 test article specifically stained 300-19/huCD37 cells and returned no staining in 300-19 cells (3 homo and negative, respectively; see FIG. 1). These results demonstrate that clone CT1 specifically targets the huCD37 antigen. (FIG. 1). FFPE sections of human normal spleen were also stained and evaluated for CD37. As shown in FIG. 2, the white pulp of the spleen (containing lymphoid follicles rich in B lymphocytes) showed strong CD37 staining, while the red pulp of the spleen (containing monocytes and red blood cells) showed little to no CD37 staining.

The immunoreactivity of each test and control article with tissues and cell pellets was determined by the consulting pathologist, Dr. David Dorfman. Samples were first categorized as either large cell, consisting of diffuse large B-cell lymphoma (DLBCL), or small cell, consisting of mantle cell lymphoma (MCL), mucosa associated lymphoid tissue (MALT), follicular lymphoma (FL), Chronic lymphocytic leukemia (CLL), small lymphocytic leukemia (SLL). The small cell group was further categorized into the respective small cell subtypes.

For each tissue evaluated, a description of the staining intensity and staining uniformity was reported. The staining intensity score and uniformity scales are described in Table 2. The final reported score for each tissue sample evaluated is the score of the test article minus the score of the respective control article. The ABC (antibodies bound per cell) level for each sample was estimated by comparing the staining score to the calibrated cell pellet controls.

TABLE 2
Intensity score and uniformity scales used to assess the immunoreactivity
of the CD37 antibody clone CT1 with FFPE tissue samples.

| | |
|---|---|
| Intensity | |
| (amount of stain) | Uniformity (number of stained cells) |

| | | | |
|---|---|---|---|
| 0 | Negative | | |
| 1 | Weak | 0 | Negative |
| 2 | Moderate | Focal | <25% |
| 3 | Strong | Heterogeneous (hetero) | 25-75% |
| 3+ | Very Strong | Homogeneous (homo) | >75% |

The levels of CD37-expression in tumor cell lines were determined using an anti-CD37 antibody-PE conjugate and the QuantiBRITE system (BD Biosciences). Several B-cell malignancy cell lines were included in the study. In order to obtain reliable ABC values, the binding experiments with an antibody-PB conjugate should be performed at a saturating concentration (concentration, at which all available binding sites are occupied by the conjugate). To determine such concentration for the anti-CD37 antibody-PE conjugate, binding experiments were performed on a panel of CD37-positive cell lines with various CD37 expression levels. The cells were incubated with a wide concentration range of PE conjugate for two hours on ice, washed with FACS buffer (PBS with 1% BSA), fixed with 1% formaldehyde in PBS and analyzed on a FACSCalibur flow cytometer (BD Biosciences). The concentration at which the conjugate saturates cell surface binding sites on all tested cell lines is determined. In subsequent binding ABC-experiments the PE conjugate was used at that concentration. Each sample was analyzed in duplicate or triplicate; several independent experiments were performed on each cell line.

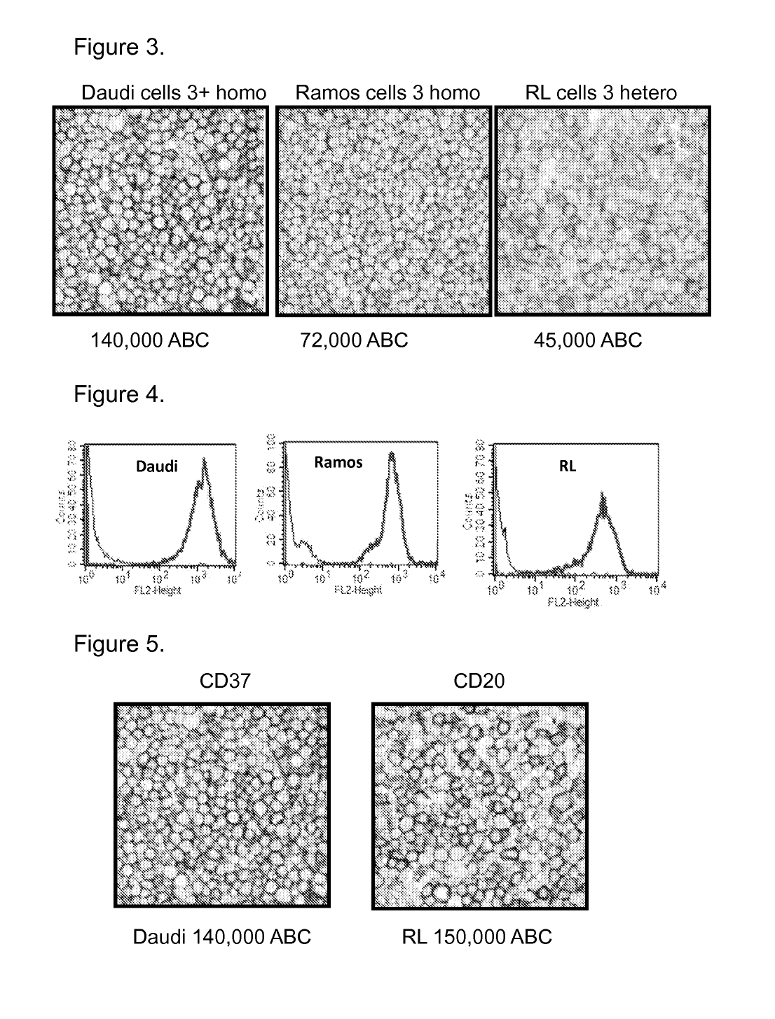

Antibodies bound per cell (ABC) values were determined for CD37 for each of the positive control cell lines, Daudi, Ramos and RL, using flow cytometry methods by staining cells with a saturating concentration of phycoerythrin (PE)-labeled anti-CD37 antibody (huCD37-PE). Immunohistochemical staining conditions were then optimized for CD37 so that Daudi, Ramos, and RL positive control cells, which each express a uniform level of CD37 and a different ABC value by flow cytometry, also showed varying levels of staining intensity by IHC (see FIG. 3 and FIG. 4). Daudi and Ramos exhibited homogeneous staining with high intensity, while staining of RL was of a lower intensity with a heterogeneous pattern. The CD37 staining intensity trends observed from the cell pellets correspond to the reported ABC values, where higher ABC values of 360,000 and 120,000 ABC resulted in more intense staining than an ABC value of 55,000. The staining results and respective ABC values are listed in Table 3. CD20 staining was optimized so that staining scores for CD20 and CD37 were similar in cell pellets expressing similar levels of CD20 and CD37, respectively (see FIG. 5).

TABLE 3
ABC values and respective staining results for CD37 on cell lines.

| | CD37 | |
|---|---|---|
| Cell Line | ABC | Score |
| Daudi | 360,000 | 3 homo |
| Ramos | 120,000 | 3 homo |
| RL | 55,000 | 3 hetero |

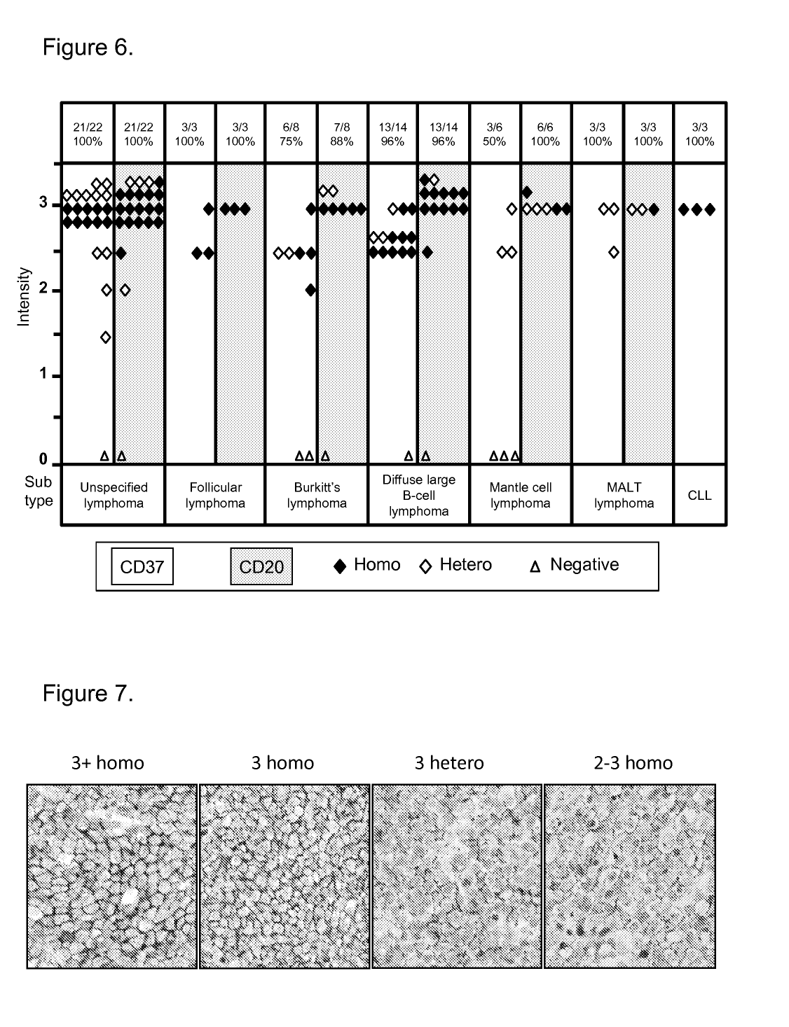

Among the neoplastic cell types evaluated, CD37 was predominantly expressed in B-NHL (Table 4). Varying levels of uniform CD37 expression were demonstrated in B-cell NHL including subtypes such as FL, MALT lymphoma, DLBCL, BL, and MCL by immunohistochemistry (see FIG. 6). As demonstrated in FIG. 6, most NHL samples show CD37 expression with an IHC score of ≧2. This corresponds to the level of staining of control cell line pellets such as Daudi, Ramos and RL with ABC values of ≧55,000. Representative photographs showing lymphoma samples stained for CD37 are shown in FIG. 7. T-cell lymphomas and Multiple Myeloma (MM) samples did not show staining for either CD37 or CD20.

TABLE 4
Summary of Staining Results of CD37 and CD20 in Neoplastic
Cell Types.

| | | # samples ≧1 | |
|---|---|---|---|
| | | hetero (%) | |
| Neoplastic Cell Type | # of samples stained | CD37 | CD20 |
| Non-Hodgkin Lymphoma | 56 | 49 (88%) | 53 (95%) |
| Hodgkin Lymphoma | 12 | 1 (8%) | 1 (8%) |
| T cell Lymphoma | 3 | 0 | 0 |
| Multiple Myeloma | 10 | 0 | 0 |

### Example 2

### Anti-Tumor Activity of huCD37-3 Antibody, and huCD37-3-SMCC-DM1 in Female CB.17 SCID Mice Bearing SU-DHL-4 (Human DLBCL) Xenografts

In this study, the anti-tumor activity (median tumor volume, mm³) of CD37-3 antibody and huCD37-3-SMCC-DM1 were evaluated in female SCID mice bearing subcutaneous SU-DHL-4 tumors, a diffuse large B-cell lymphoma model. Mice were randomized into groups (n=10 per group) by body weight. Treatment was the day after randomization, and groups included a control group dosed with PBS (200 μL/injection), huCD37-3 antibody, and non-cleavable huCD37-3-SMCC-DM1 conjugate. Treatments were administered as single intravenous bolus of 10 mg of protein/kg/injection for antibody and huCD37-3-SMCC-DM1. All treatments were well tolerated with no body weight loss as with PBS control animals. Ten out of ten PBS control animals developed tumors (100% tumor take rate), reaching a median tumor volume of 1000 mm³ on 37 days post cell inoculation. The mean and median tumor volumes were calculated for each treatment group. In addition, for each treatment a % TIC value was calculated which corresponds to the median tumor volume of each treated group divided by the median tumor volume of the vehicle treated group, A treatment with a % TIC value at or below 42% is considered active, while a treatment with a % TIC value at or below 12% is considered highly active.

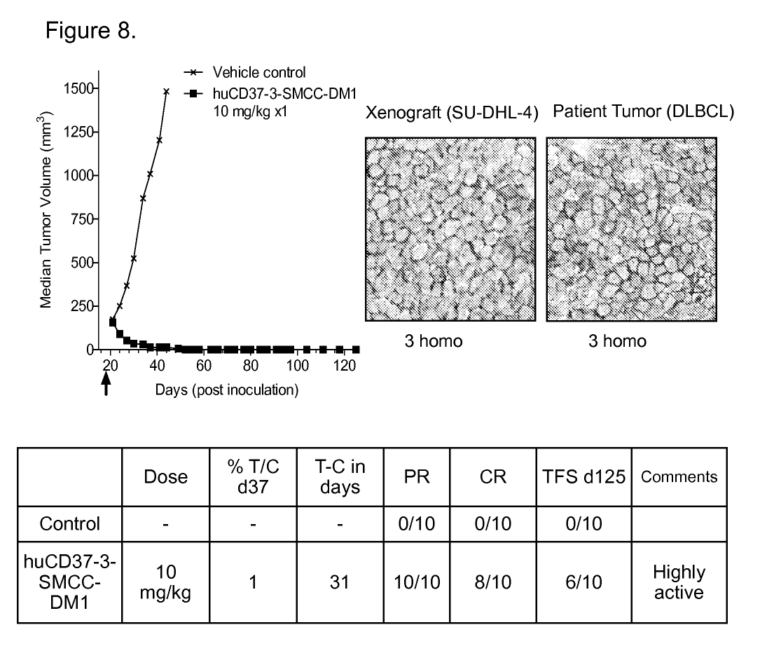

Single intravenous treatment of huCD37-3 antibody (10 mg/kg/injection) was active in this study with a 32% T/C; however there were no tumor-free survivors (TFS), huCD37-3-SMCC-DM1 (10 mg/kg/injection) was highly active with % DC of 1%, Anti-tumor activity (median tumor volume, mm³) of huCD37-3-SMCC-DM1 only as a single intravenous treatment (10 mg/kg/injection) is shown in FIG. 8.

An additional study was conducted to evaluate CD37-3 antibody and huCD37-3-SMCC-DM1 in female SCID mice bearing subcutaneous SU-DHL-4 tumors as outlined in Example 17 of (U.S. Published Application No. 2011/0256153, herein incorporated by reference). Animals were randomized by body weight into treatment groups and treated once on day 15 post cell inoculation with either 10 mg/kg of huCD37-3 Ab or huCD37-3-SMCC-DM1. The % T/C value on day 38 post cell inoculation corresponded to 34% or 4% for huCD37-3 or huCD37-3-SMCC-DM1, respectively. On day 74 post cell inoculation, huCD37-3-SMCC-DM1 treatment resulted in 8 of 10 tumor-free survivors. No TFS were observed in the huCD37-3 antibody or PBS vehicle control groups. The huCD37-3-SMCC-DM1 conjugate also showed strong efficacy at single doses of 2.5 or 5 mg/kg in this model with % T/C values on day 37 post cell innoculation of 18% and 6%, respectively. Therefore, huCD37-3-SMCC-DM1 was highly active in the SU-DHL-4 model at a single dose of 10 mg/kg and was active at a single dose of 2.5 mg/kg.

### Example 3

### Anti-Tumor Efficacy of huCD37 Antibody huCD37-3, huCD37-3-SMCC-DM1 and Standard-of-Care Chemotherapeutics in SCID Mice Bearing DoHH2 Xenografts, a Human Follicular Lymphoma Model

In this study, the antitumor activity of huCD37-3 antibody, huCD37-3-SMCC-DM1, Rituximab® antibody, and CVP (cyclophosphamide, vincristine and prednisone) were investigated in female SCID mice bearing subcutaneous DoHH2 tumors, a human follicular lymphoma model. Eleven days after tumor cell inoculation eighty-one mice were randomized into 9 groups (n=9 per group) by tumor volume. Treatment was initiated the day after randomization (Day 12), and groups included a control group dosed with PBS (200 μL/injection), six groups administered single intravenous doses of either huCD37-3 antibody or huCD37-3-SMCC-DM1 conjugate at 10, 5, or 2.5 mg/kg, one group administered six intravenous Rituximab antibody doses at 2 mg/kg, twice per week times three, and one group administered a single intravenous dose of cyclophosphamide at 40 mg/kg and vincristine at 0.5 mg/kg, along with five daily oral doses of prednisone at 0.2 mg/kg.

Single treatments at 10, 5, and 2.5 mg/kg of huCD37-3 antibody and huCD37-3-SMCC-DM1 conjugate as well as rituximab antibody at 2 mg/kg/injection, 2qw×3, were well tolerated with no body weight loss as with PBS control animals. Treatment with CVP was toxic with one animal related drug death and 12% mean body weight loss (day 18 nadir). This animal was excluded from data analysis.

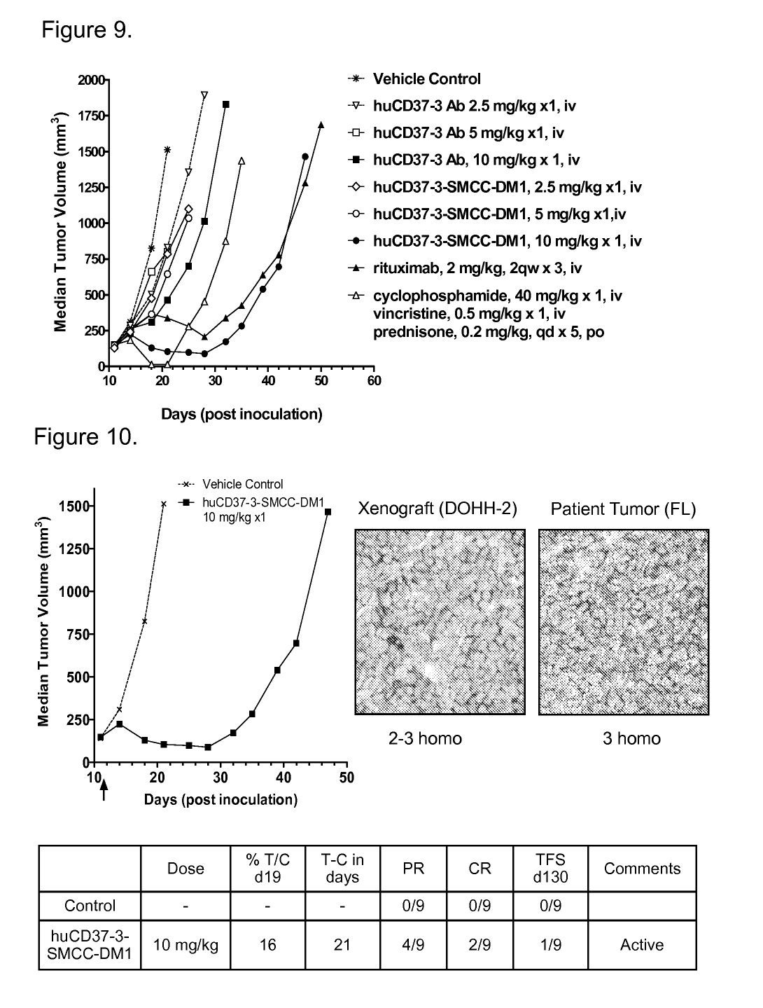

Nine out of nine PBS control animals developed tumors (100% tumor take rate), reaching a median tumor volume of 800 mm³ on 19 days post cell inoculation. The median and mean tumor volume was calculated for each treatment group. Single intravenous treatments with 2.5 and 5 mg/kg of huCD37-3 antibody and huCD37-3-SMCC-DM1 conjugate, as well as, Rituximab® antibody (2 mg/kg, twice weekly time three) were inactive in this study with >42% T/Cs. Single intravenous treatment with 10 mg/kg of huCD37-3 antibody and huCD37-3-SMCC-DM1 conjugate were active with 37% and 16% T/C, respectively. Treatment with the doses and schedule of CVP administered, although highly active with 2% T/C, was above the tolerated dose for this regimen. Anti-tumor activity (median tumor volume, mm³) of huCD37-3 antibody, huCD37-3-SMCC-DM1, and standard chemotherapeutics in SCID mice bearing DoHH2 human tumor xenografts are shown in FIG. 9. Anti-tumor activity (median tumor volume, mm³) of huCD37-3-SMCC-DM1 only as a single intravenous treatment (10 mg/kg/injection) in this model is shown in FIG. 10.

### Example 4

### Anti-Tumor Efficacy of Antibody huCD37-3, huCD37-3-SMCC-DM1, and Standard of Care Agents in SCID Mice Bearing JVM-3 Xenografts, a Human B Cell Chronic Lymphocytic Leukemia (CLL) Model

In this study, the anti-tumor activity of huCD37-3 antibody, the non-cleavable conjugate huCD37-3-SMCC-DM1, and two standard-of-care agents: Ofatumumab, an antibody targeting CD20, and Bendamustine, a chemotherapeutic agent, was evaluated in a human B cell chronic lymphocytic leukemia model using JVM-3 (CD37+/CD20+) cells implanted subcutaneously in female SCID mice. Six days after tumor cell inoculation ninety mice were randomized into nine groups (n=10 per group) by tumor volume. Treatment was initiated the day after randomization, and groups included a control group dosed with PBS (200 μL/injection), huCD37-3 antibody, non-cleavable huCD37-3-SMCC-DM1 conjugate, Ofatumumab antibody and Bendamustine. Treatments were administered as a single intravenous dose of 10, 5, and 2.5 mg/kg for huCD37-3 antibody and huCD37-3-SMCC-DM1, at 5 mg/kg, twice weekly times three for Ofatumumab and as a single dose of 50 mg/kg for Bendamustine.

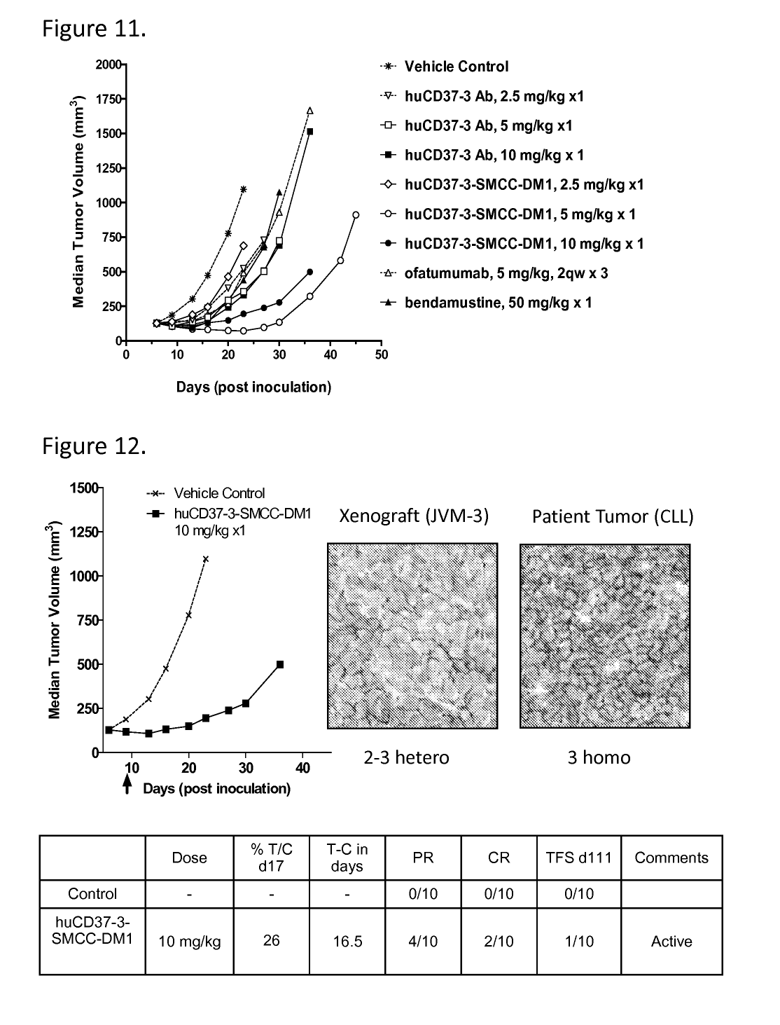

Single treatments at 10, 5, and 2.5 mg/kg of huCD37-3 antibody and huCD37-3-SMCC-DM1 conjugate as well as Ofatumumab antibody at 5 mg/kg/injection, 2qw×3, were well tolerated with no body weight loss as with PBS control animals. Treatment with Bendamustine was tolerated with 8% mean body weight loss (day 9 nadir). Ten out of ten PBS control animals developed tumors (100% tumor take rate), reaching a median tumor volume of 500 mm³ on 16 days post cell inoculation. The median and mean tumor volume was calculated for each treatment group. The results were plotted against days post inoculation for mean tumor volume and for median tumor volume. Treatment with standard of care agents Ofatumumab (5 mg/kg, twice weekly time three) and Bendamustine (single intravenous dose of 50 mg/kg) were active in this study with 39% and 31% T/C, respectively. Single intravenous treatments with 2.5 mg/kg of huCD37-3 antibody and huCD37-3-SMCC-DM1 conjugate were inactive in this study with >42% T/Cs. Single intravenous treatment with 10 mg/kg of huCD37-3 antibody and huCD37-3-SMCC-DM1 conjugate were active with 29% and 26% T/C, respectively, and treatment with an intermediate dose (5 mg/kg single injection) of huCD37-3 antibody and huCD37-3-SMCC-DM1 conjugate were active with 31% and 16% T/C, respectively. Anti-tumor activity (median tumor volume, mm³) of huCD37-3 antibody, huCD37-3-SMCC-DM1, Ofatumumab, and Bendamustine in SCID mice bearing JVM-3 human tumor xenografts is shown in FIG. 11. Anti-tumor activity (median tumor volume, mm³) of huCD37-3-SMCC-DM1 only as a single intravenous treatment (10 mg/kg/injection) in this model is shown in FIG. 12.

### Example 5

### Correlation of Anti-Tumor Activity (Median Tumor Volume, Mm³) of huCD37-3-SMCC-DM1 as a Single Intravenous Treatment (10 mg/Kg/Injection) in Xenograft Models with IHC Staining Scores

The antitumor effect of huCD37-3-SMCC-DM1 was evaluated in three lymphoma xenograft models as described in Examples 2, 3, and 4 and correlated with respective CD37 expression, determined by IHC as described in Example 1. FFPE samples prepared from mouse xenograft tumor models were evaluated for CD37 positivity using the manual assay method described in Example 1. FFPE mouse xenograft tissues derived from the following cell lines showed the following staining patterns: SU-DHL-4 showed homogeneous staining patterns with level 3 intensity; DOHH-2 showed homogeneous staining patterns with levels 2 and 3 intensity; JVM-3 showed heterogenous patterns with level 2 and 3 intensity. Representative photographs of tumor xenografts and respective in vivo activity from a single intravenous treatment of huCD37-3-SMCC-DM1 (10 mg/kg/injection) are shown in FIGS. 8, 10 and 12. A summary of the in vivo activity and respective CD37 staining scores for each xenograft are listed in Table 5. Among the three xenografts evaluated, the tumor with highest expression (3 homo) also showed the highest activity when treated with huCD37-3-SMCC-DM1. The other 2 models showed lower expression and lower activity.

TABLE 5
In vivo activity of huCD37-3-SMCC-DM1 and respective
CD37 staining scores in xenograft models.

| | Activity from a single | |
|---|---|---|
| | intravenous treatment (10 | CD37 IHC |
| Xenograft Model | mg/kg/injection) | staining score |
| SU-DHL-4 | Highly Active | 3 homo |
| (derived from human | | |
| DLBCL) | | |
| DOHH-2 | Active | 2-3 homo |
| (derived from human FL) | | |
| JVM-3 | Active | 2-3 hetero |
| (derived from human CLL) | | |

### Example 6

### Immunohistochemical Staining of CD37 in Formalin Fixed Paraffin Embedded (FFPE) Samples-Automated Methods

The IHC staining assay used the murine anti-CD37 antibody clone CT1 (Leica Cat# NCL-CD37) as the test article and murine IgG1 isotype control (Leica, Cat# MOPC21 AB) and was performed on the Leica Bond RX automated stainer. Paraffin wax was removed when slides containing samples were baked at 60° C. Then the excess paraffin wax was removed with Dewax solution (Leica). Following antigen retrieval in pH 6.0 Bond Epitope Retrieval Solution 1 (ER1, Leica), sections were blocked in 3-4% peroxide. To prepare working solutions of primary antibodies, the CD37-test and isotype control articles were each diluted to 4.2 μg/mL in antibody diluent. Slides were incubated with the test article (anti-CD37), or control article (muIgG1), followed by incubation with the post primary reagent (rabbit anti-mouse IgG, Leica), then by incubation with a polymer (goat anti-rabbit-HRP-IgG, Leica). Slides were developed by incubation with DAB (3,3-diaminobenzidine tetrahydrochloride, Leica) resulting in the color signal. Slides containing developed tissue sections were counterstained with Hematoxylin (Leica) then cleared of excess stain and dehydrated through a series of 95% and 100% ETOH immersion, followed by immersion in Xylene. Coverslips were mounted onto the slides using mounting medium (Richard Allan Scientific).

All stained samples were evaluated and scored. Control samples were first evaluated followed by test samples (whole sections and individual cores from the tissue micro arrays). For each tumor tissue or cell pellet evaluated, a description of the staining intensity and respective proportion of tumor cells stained was reported. Membrane associated staining was recorded for every sample. When duplicate scores were evaluated from one patient, only the higher score was included in the analysis. If the score describes only cytoplasmic staining, then the final score was reported as zero (0). Intensity and uniformity scores were given to each sample as described in Table 6. Staining intensity and distribution patterns were scored relative to control IgG staining (non-specific). Intensity was scored on a scale of 0 to 3 (0=no staining, 1=weak, 2=moderate and 3=strong), and distribution was scored as focal (<25% of cells stained), heterogeneous (25-75% of cells stained), and homogeneous (>75% of cells stained). In normal tissue, only the defined substructures were evaluated when calculating intensity and proportion.

TABLE 6
IHC Scoring System Consisting of Intensity and Uniformity Scales

| | |
|---|---|
| Intensity | |
| (amount of stain) | Uniformity (number of stained cells) |

| | | | |
|---|---|---|---|
| 0 | Negative | 0 | Negative |
| 1 | Weak | Focal | <25% |
| 2 | Moderate | Heterogeneous (hetero) | 25-75% |
| 3 | Strong | Homogeneous (homo) | >75% |

FFPE tumor samples were derived from tumor micro arrays, as well as human tissue blocks.

Cells (tumor cells or transfected cells) were formalin fixed and paraffin embedded (FFPE). FFPE cell pellet samples shown to exhibit varying ranges of CD37 expression by flow cytometry (Daudi, Ramos, Namalwa, and Colo205), normal human tissues (spleen and tonsil), and Non-Hodgkin's lymphoma tissue samples were used to characterize positive and negative controls and for analysis of specificity.

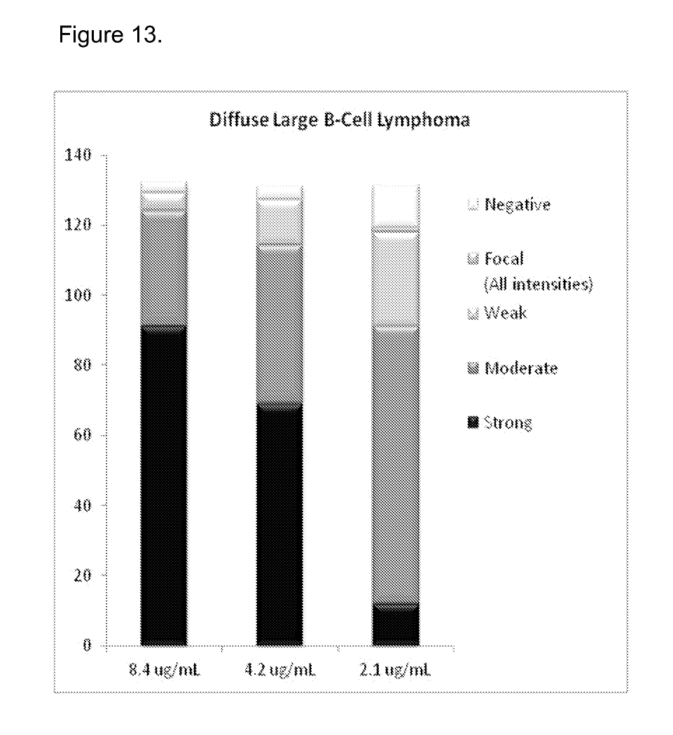

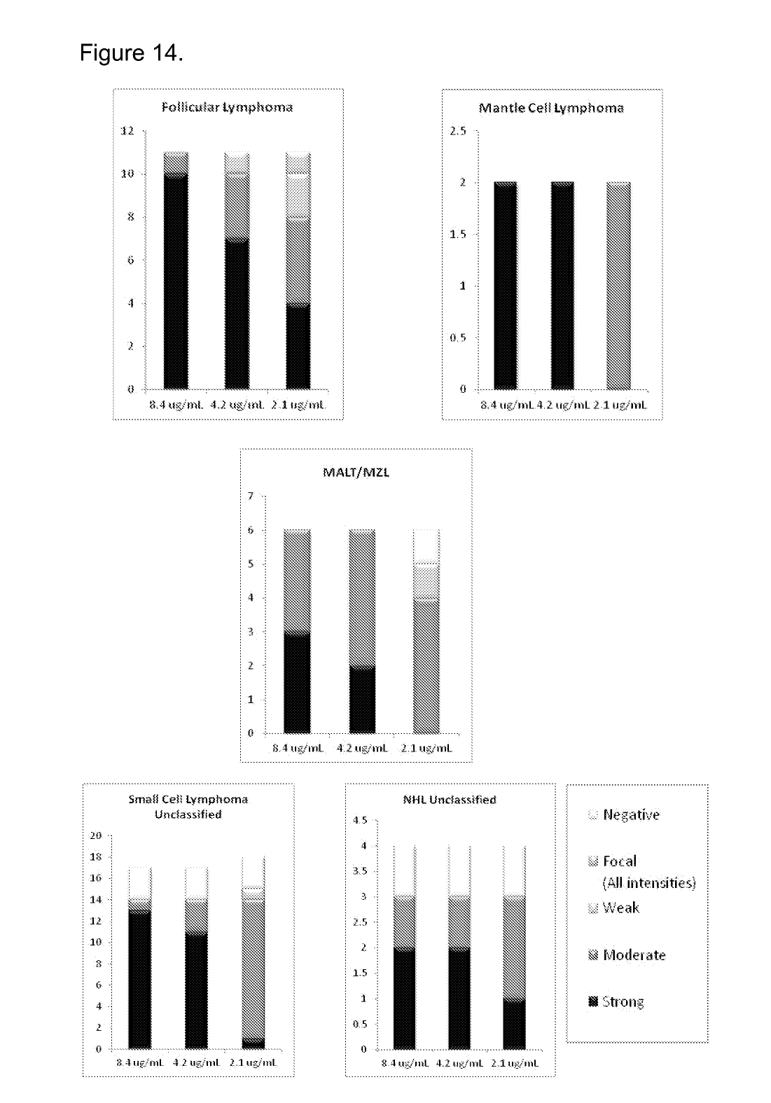

To determine assay conditions, a range of dilutions of test and control article was tested to select conditions that exhibit an appropriate level of sensitivity. Experiments were performed on a panel of FFPE samples including CD37-positive cell pellets, normal human tissue, and tissue micro arrays consisting of CD37 positive Non-Hodgkin's lymphoma samples. Each sample was stained with a serial dilution of test article concentrations (2.1, 4.2, and 8.4 μg/mL) or control article. All samples were evaluated and scored by a board certified pathologist and categorized as either small cell (follicular lymphoma [FL], Mantle cell lymphoma [MCL], MALT type marginal zone B cell lymphoma, marginal zone B cell lymphoma, unclassified small cell lymphoma, unclassified non-hodgkin's lymphoma [NHL]) or large cell (DLBCL). The relative staining intensities for each dilution were compared for each sample to identify the optimal dilution. The criteria for optimal dilution was a dilution which 1) caused no background staining in samples stained with isotype control 2) caused no staining in negative tissue controls stained with test article and 3) differentiated between varying levels of membrane-associated CD37 expression among test samples representing the indication of interest (e.g., DLBCL, FL, CLL). The test article concentration of 4.2 μg/mL was experimentally identified as an optimal dilution and is therefore a particularly useful concentration. A summary of staining results for large cell lymphoma samples is shown in Table 7, and a graph of the scoring distribution for each concentration is shown in FIG. 13. A summary of staining results for small cell lymphoma samples is listed in Table 8, and a graph of the scoring distribution for each concentration is depicted in FIG. 14.

TABLE 7
Staining distribution in large cell lymphoma samples

| | No. of Samples in Each Staining | | |
|---|---|---|---|
| | Category | | |
| Scores | 8.4 μg/mL | 4.2 μg/mL | 2.1 μg/mL |
| 3 homo | 82 | 51 | 4 |
| 2-3 homo | 9 | 18 | 8 |
| 2 homo | 24 | 29 | 53 |
| 2 hetero | 0 | 2 | 0 |
| 1-2 homo | 9 | 13 | 22 |
| 1-2 hetero | 0 | 1 | 4 |
| 1 homo | 5 | 11 | 12 |
| 1 hetero | 0 | 2 | 4 |
| 0-1 homo | 0 | 0 | 7 |
| 0-1 hetero | 0 | 0 | 4 |
| 1 focal | 0 | 0 | 1 |
| 0 | 3 | 4 | 12 |
| Total | 135 | 135 | 135 |

TABLE 8
Staining distribution in small cell lymphoma samples at 3 concentrations

| | | | | | | | | | | Small Cell | | | Unclassified | | |
|---|---|---|---|---|---|---|---|---|---|---|---|---|---|---|---|
| | FL | | | MALT | | | MCL | | | unclassified | | | NHL | | |
| | Concentration (μg/Ml) | | | | | | | | | | | | | | |
| Scores | 8.4 | 4.2 | 2.1 | 8.4 | 4.2 | 2.1 | 8.4 | 4.2 | 2.1 | 8.4 | 4.2 | 2.1 | 8.4 | 4.2 | 2.1 |
| 3 homo | 8 | 6 | 2 | 3 | — | — | 2 | 2 | — | 12 | 6 | — | 2 | 2 | 1 |
| 2-3 homo | 2 | 1 | 1 | — | 2 | — | — | — | — | 1 | 5 | 1 | — | — | — |
| 1-3 homo | — | — | 1 | — | — | — | — | — | — | — | — | — | — | — | — |
| 2 homo | 1 | 3 | 1 | 2 | 3 | 1 | — | — | — | 1 | 3 | 9 | 1 | 1 | 1 |
| 2 hetero | — | — | — | — | — | — | — | — | — | — | — | — | — | — | 1 |
| 1-2 homo | — | — | 3 | — | — | 1 | — | — | — | — | — | 3 | — | — | — |
| 1-2 hetero | — | — | — | 1 | 1 | 2 | — | — | 2 | — | — | 1 | — | — | — |
| 1 homo | — | 1 | 1 | — | — | — | — | — | — | — | — | — | — | — | — |
| 1 hetero | — | — | — | — | — | 1 | — | — | — | — | — | 1 | — | — | — |
| 0-1 homo | — | — | 1 | — | — | — | — | — | — | — | — | — | — | — | — |
| 1 focal | — | — | 1 | — | — | — | — | — | — | — | — | — | — | — | — |
| 0 | — | — | — | — | — | 1 | — | — | — | 3 | 3 | 3 | 1 | 1 | 1 |

| | | | | | |
|---|---|---|---|---|---|
| Total | 11 | 6 | 2 | 17/17/18 | 4 |

### Example 7

### Identification and Characterization of Controls that Characterize the Dynamic Range of the Assay-Automated Staining Methods

Quality Controls:

The mantle zone and the marginal zone of normal human spleen and germinal zone and mantle zone of the tonsil were used as positive controls in each assay to verify that the staining procedure performed as expected. The interfollicular areas of normal human tonsil and the red pulp of human normal spleen were used as negative controls. These controls were used as assay verification controls during the optimization and validation phases. These results were reviewed to confirm that the selected controls give consistent results and to confirm they spanned the dynamic range of the assay. Exemplary staining scores for the quality controls are listed in Table 9 at four staining concentrations (2.1, 4.2, 8.4, and 16.7 μg/mL) and indicate that the concentration of primary antibody did affect staining results of tested samples.

TABLE 9

| | Isotype | | | | |
|---|---|---|---|---|---|
| | control | CD37 Antibody | | | |
| | (16.7 | (1:20, 16.7 | (1:40,8.4 | (1:80, 4.2 | (1:160, 2.1 |
| Control | μg/mL) | μg/mL) | μg/mL) | μg/mL) | μg/mL) |
| Sample | D7 | D1 | D2 | D4 | D6 |
| Daudi cell pellet | 0 | 3 homo | 3 homo | 3 homo | 2 homo |
| Ramos cell pellet | 0 | 3 homo | 2 homo | 2 homo | 2 hetero |
| RL cell pellet | 0 | 2 hetero | 2 hetero | 1-2 hetero | 1 focal |
| Namalwa cell pellet | 0 | 2 hetero | 2 hetero | 2 hetero | 1 focal |
| Colo205 cell pellet | 0 | 0 | 0 | 0 | 0 |
| Normal human | MG = 0 | MG = 3 | MG = 3 | MG = 2 | MG = 1-2 |
| spleen | | homo | homo | homo | homo |
| | MT/F = 0 | MT/F = 3 | MT/F = 3 | MT/F = 3 | MT/F = 2 |
| | | homo | homo | homo | homo |
| | RP = 0 | RP = 0 | RP = 0 | RP = 0 | RP = 0 |
| Normal human | GC = 0 | GC = 3 | GC = 3 | GC = 3 | GC = 3 |
| tonsil | | homo | homo | homo | homo |
| | MT = 0 | MT = 3 | MT = 3 | MT = 3 | MT = 2 |
| | | homo | homo | homo | homo |
| | IF = 0 | IF = 0 | IF = 0 | IF = 0 | IF = 0 |

MG = marginal zone;
MT = mantle zone;
IF = interfollicular;
F = follicle;
GC = germinal center;
RP = red pulp

### Example 8

### Performance Analysis of the Automated Staining Method

The intended use of this assay is to specifically detect CD37 reproducibly and with the appropriate sensitivity to differentiate varying levels and uniformity of membrane-associated CD37 expression (optimal dynamic range) in B-cell malignancies. Therefore, specificity, reproducibility, and sensitivity are considered as performance criteria.

The specificity and sensitivity of the study assay was characterized by staining and evaluating a panel of tissue micro arrays. Staining was observed to confirm that positive staining is consistently localized to the tumor tissue with normal adjacent tissue components including stroma, blood vessels, and normal organ tissue staining negative or positive as expected. For each subtype of B-cell malignancy, the distribution of staining scores among tissue micro arrays was observed. A similar distribution of scores suggests the method performed well and was not overly sensitive to minor variations in various fixation and processing conditions. The precision of the study assay was also investigated by evaluating intra-run and inter-run reproducibility of the assay using FFPE samples consisting of cell pellets that express varying levels of CD37: Daudi (high CD37 expression), Ramos (intermediate CD37 expression), Namalwa (low CD37 expression), and Colo205 (negative). Additionally, normal human spleen (two samples), normal human tonsil, and a marginal zone lymphoma sample were included. For intra-run reproducibility, nine slides each containing a section of each control were placed at random locations the Leica Bond RX. For inter-run reproducibility, three slides containing sections from the same samples were stained on three different days. All slides from both intra-run and inter-run reproducibility experiments were evaluated and found to be reproducible.

### Example 9

### A CD37 Staining Score by IHC Correlates with Activity of huCD37-3-SMCC-DM1

Potency and specificity of huCD37-3-SMCC-DM1 is analyzed against CD37-positive cell lines with a wide range of CD37 expression. The level of CD37 expression is determined by flow cytometry as described in Example 1. Alternatively, the level of CD37 expression is determined by IHC using a manual staining method as described in Example 1 or by IHC using an automated staining method as described in Examples 6-8. Potency is evaluated using suitable in vivo xenograft models as described in Examples 2-4. Dosing of huCD37-3-SMCC-DM1 is done intravenously at doses such as 10 mg/kg, 5 mg/kg and/or 2.5 mg/kg. In addition, suitable CD37-positive cells with low CD37 expression levels can be used such as, for example, Namalwa. To verify specificity of activity, CD37-negative cell lines, such as Colo205, can be included in the experiments.

All publications, patents, patent applications, internet sites, and accession numbers/database sequences (including both polynucleotide and polypeptide sequences) cited herein are hereby incorporated by reference in their entirety for all purposes to the same extent as if each individual publication, patent, patent application, internet site, or accession number/database sequence were specifically and individually indicated to be so incorporated by reference.

### SEQUENCES

SEQ ID NO: 1
human CD37
MSAQESCLSLIKYFLFVFNLFFFVLGSLIFCFGIWILIDKTSFVSFVGL
AFVPLQIWSKVLAISGIFTMGIALLGCVGALKELRCLLGLYFGMLLLLF
ATQITLGILISTQRAQLERSLRDVVEKTIQKYGTNPEETAAEESWDYVQ
FQLRCCGWHYPQDWFQVLILRGNGSEAHRVPCSCYNLSATNDSTILDKV
ILPQLSRLGHLARSRHSADICAVPAESHIYREGCAQGLQKWLHNNLISI
VGICLGVGLLELGFMTLSIFLCRNLDHVYNRLAYR

### Variable Heavy Chain CDR Amino Acid Sequences:

| Antibody | VH-CDR1 | VH-CDR2 | VH-CDR3 |
|---|---|---|---|
| CD37-3 | TSGVS | VIWGDGSTN | GGYSLAH |
| | (SEQ ID | (SEQ ID | (SEQ ID |
| | NO: 4) | NO: 5) | NO: 6) |
| CD37-12 | KYGMN | WINTNTGESR | GTVVAD |
| | (SEQ ID | (SEQ ID | (SEQ ID |
| | NO: 7) | NO: 8) | NO: 9) |
| CD37-38 | SGFGWH | YILYSGGTD | GYYGYGAWFVY |
| | (SEQ ID | (SEQ ID | (SEQ ID |
| | NO: 10) | NO: 11) | NO: 12) |
| CD37-50 | SGFAWH | YILYSGSTV | GYYGYGAWFAY |
| | (SEQ ID | (SEQ ID | (SEQ ID |
| | NO: 13) | NO: 14) | NO: 15) |
| CD37-51 | SGFAWH | YIHYSGSTN | GYYGFGAWFVY |
| | (SEQ ID | (SEQ ID | (SEQ ID |
| | NO: 16) | NO: 17) | NO: 18) |
| CD37-56 | SGFAWH | YIHYSGGTN | GYYGFGAWFAY |
| | (SEQ ID | (SEQ ID | (SEQ ID |
| | NO: 19) | NO: 20) | NO: 21) |
| CD37-57 | SGFAWH | YILYSGSTV | GYYGYGAWFAY |
| | (SEQ ID | (SEQ ID | (SEQ ID |
| | NO: 22) | NO: 23) | NO: 24) |
| CONSEN- | SGF[A | YI[L or H] | GYYG[Y |
| SUS | or G] | YSG [G | or F] |
| | WH | or S]T | GAWF[V |
| | (SEQ ID | [D, V, | or A]Y |
| | NO: 25) | or N] | (SEQ ID |
| | | (SEQ ID | NO: 27) |
| | | NO: 26) | |

### Variable Light Chain CDR Amino Acid Sequences

| Antibody | VL-CDR1 | VL-CDR2 | VL-CDR3 |
|---|---|---|---|
| CD37-3 | RASENIRSNLA | VATNLAD | QHYWGTTWT |
| | (SEQ ID | (SEQ ID | (SEQ ID |
| | NO: 28) | NO: 29) | NO: 30) |
| CD37-12 | RASQSVSTSS | YASNLAS | QHSWEIPYT |
| | YSYLY | (SEQ ID | (SEQ ID |
| | (SEQ ID | NO: 32) | NO: 33) |
| | NO: 31) | | |
| CD37-38 | SASSSVTYMH | DTSKLAS | QQWISNPPT |
| | (SEQ ID | (SEQ ID | (SEQ ID |
| | NO: 34) | NO: 35) | NO: 36) |
| CD37-50 | SATSSVTYMH | DTSKLPY | QQWSDNPPT |
| | (SEQ ID | (SEQ ID | (SEQ ID |
| | NO: 37) | NO: 38) | NO: 39) |
| | | Humanized | |
| | | DTSNLPY | |
| | | (SEQ ID | |
| | | NO: 40) | |
| CD37-51 | SATSSVTYMH | DTSKLAS | QQWSSNPPT |
| | (SEQ ID | (SEQ ID | (SEQ ID |
| | NO: 41) | NO: 42) | NO: 43) |
| CD37-56 | SASSSVTYMH | DTSKLAS | QQWISDPPT |
| | (SEQ ID | (SEQ ID | (SEQ ID |
| | NO: 44) | NO: 45) | NO: 46) |
| | | Humanized | |
| | | DTSNLAS | |
| | | (SEQ ID | |
| | | NO: 47) | |
| CD37-57 | SATSSVTYMH | DTSKLAS | QQWSDNPPT |
| | (SEQ ID | (SEQ ID | (SEQ ID |
| | NO: 48) | NO: 49) | NO: 50) |
| | | Humanized | |
| | | DTSNLAS | |
| | | (SEQ ID | |
| | | NO: 51) | |
| CONSENSUS | SA[T or S] | DTS[K or N] | QQW[I or S] |
| | SSVTYMH | L[A or P] | [S or D] |
| | (SEQ ID | [S or Y] | [N or D] |
| | NO: 52) | (SEQ ID | PPT |
| | | NO: 53) | (SEQ ID |
| | | | NO: 54) |

### Variable Heavy Chain Amino Acid Sequences

| Antibody | VH Amino Acid Sequence (SEQ ID NO) |
|---|---|
| muCD37-3 | QVQVKESGPGLVAPSQSLSITCTVSGFSLT |
| | TSGVSWVRQPPGKGLEWLGVIWGDGSTNYH |
| | SALKSRLSIKKDHSKSQVFLKLNSLQTDDT |
| | ATYYCAKGGYSLAHWGQGTLVTVSA |
| | (SEQ ID NO: 55) |
| chCD37-3 | QVQVKESGPGLVAPSQSLSITCTVSGFSLT |
| | TSGVSWVRQPPGKGLEWLGVIWGDGSTNYH |
| | SALKSRLSIKKDHSKSQVFLKLNSLQTDDT |
| | ATYYCAKGGYSLAHWGQGTLVTVSA |
| | (SEQ ID NO: 56) |
| huCD37-3v1.0 | QVQVQESGPGLVAPSQTLSITCTVSGFSLT |
| | TSGVSWVRQPPGKGLEWLGVIWGDGSTNYH |
| | PSLKSRLSIKKDHSKSQVFLKLNSLTAADT |
| | ATYYCAKGGYSLAHWGQGTLVTVSS |
| | (SEQ ID NO: 57) |
| huCD37-3v1.1 | QVQVQESGPGLVAPSQTLSITCTVSGFSLT |
| | TSGVSWVRQPPGKGLEWLGVIWGDGSTNYH |
| | SSLKSRLSIKKDHSKSQVFLKLNSLTAADT |
| | ATYYCAKGGYSLAHWGQGTLVTVSS |
| | (SEQ ID NO: 58) |
| muCD37-12 | QIQLVQSGPELKKPGETVKISCKASGYTFT |
| | KYGMNWVKQAQGKGLKWMGWINTNTGESRN |
| | AEEFKGRFAFSLETSASTAYLQINNLKYED |
| | TATYFCGRGTVVADWGQGTTLTVSS |
| | (SEQ ID NO: 59) |
| chCD37-12 | QIQLVQSGPELKKPGETVKISCKASGYTFT |
| | KYGMNWVKQAQGKGLKWMGWINTNTGESRN |
| | AEEFKGRFAFSLETSASTAYLQINNLKYED |
| | TATYFCGRGTVVADWGQGTTLTVSS |
| | (SEQ ID NO: 60) |
| muCD37-38 | DVQLQESGPDLVKPSQSLSLTCTVTGYSIT |
| | SGFGWHWIRQFPGNKLEWMAYILYSGGTDY |
| | NPSLKSRISITRDTSKNQFFLRLSSVTTED |
| | TATYYCARGYYGYGAWFVYWGQGTLVTVSA |
| | (SEQ ID NO: 61) |
| chCD37-38 | QVQLQESGPDLVKPSQSLSLTCTVTGYSIT |
| | SGFGWHWIRQFPGNKLEWMAYILYSGGTDY |
| | NPSLKSRISITRDTSKNQFFLRLSSVTTED |
| | TATYYCARGYYGYGAWFVYWGQGTLVTVSA |
| | (SEQ ID NO: 62) |
| huCD37-38 | QVQLQESGPGLVKPSQSLSLTCTVSGYSIT |
| | SGFGWHWIRQFPGKGLEWMAYILYSGGTDY |
| | NPSLKSRISITRDTSKNQFFLRLSSVTAAD |
| | TATYYCARGYYGYGAWFVYWGQGTLVTVSS |
| | (SEQ ID NO: 63) |
| muCD37-50 | DVQLQESGPDLLKPSQSLSLTCTVTGYSIT |
| | SGFAWHWIRQFPGNKLEWMGYILYSGSTVY |
| | SPSLKSRISITRDTSKNHFFLQLNSVTTED |
| | TATYYCARGYYGYGAWFAYWGQGTLVTVSA |
| | (SEQ ID NO: 64) |
| huCD37-50 | QVQLQESGPGLLKPSQSLSLTCTVSGYSIT |
| | SGFAWHWIRQHPGNKLEWMGYILYSGSTVY |
| | SPSLKSRISITRDTSKNHFFLQLNSVTAAD |
| | TATYYCARGYYGYGAWFAYWGQGTLVTVSA |
| | (SEQ ID NO: 65) |
| muCD37-51 | DVQLQESGPDLLKPSQSLSLTCTVTGYSIS |
| | SGFAWHWIRQFPGNKLEWMGYIHYSGSTNY |
| | SPSLKSRISITRDSSKNQFFLQLNSVTTED |
| | TATYYCARGYYGFGAWFVYWGQGTLVTVSA |
| | (SEQ ID NO: 66) |
| huCD37-51 | EVQLVESGPEVLKPGESLSLTCTVSGYSIS |
| | SGFAWHWIRQFPGKGLEWMGYIHYSGSTNY |
| | SPSLQGRISITRDSSINQFFLQLNSVTASD |
| | TATYYCARGYYGFGAWFVYWGQGTLVTVSA |
| | (SEQ ID NO: 67) |
| muCD37-56 | DVQLQESGPDLVKPSQSLSLTCTVTGYSIT |
| | SGFAWHWIRQFPGNKLEWMGYIHYSGGTNY |
| | NPSLKSRVSITRDTSKNQFFLQLNSVTTED |
| | TATYYCARGYYGFGAWFAYWGQGTLVPVSA |
| | (SEQ ID NO: 68) |
| huCD37-56 | QVQLQESGPGLVKPSQSLSLTCTVSGYSIT |
| | SGFAWHWIRQFPGKGLEWMGYIHYSGGTNY |
| | NPSLKSRVSITRDTSKNQFFLQLNSVTAAD |
| | TATYYCARGYYGFGAWFAYWGQGTLVPVSA |
| | (SEQ. ID NO: 69) |
| muCD37-57 | DVQLQESGPDLLKPSQSLSLTCTVTGYSIT |
| | SGFAWHWIRQFPGNKLEWMGYILYSGSTVY |
| | SPSLKSRISITRDTSKNQFFLQLNSVTTED |
| | TATYYCARGYYGYGAWFAYWGQGTLVTVSA |
| | (SEQ ID NO: 70) |
| huCD37-57 | QVQLQESGPGLLKPSQSLSLTCTVSGYSIT |
| | SGFAWHWIRQFPGKGLEWMGYILYSGSTVY |
| | SPSLKSRISITRDTSKNQFFLQLNSVTAAD |
| | TATYYCARGYYGYGAWFAYWGQGTLVTVSA |
| | (SEQ ID NO: 71) |

### Variable Light Chain Amino Acid Sequences

| Antibody | VL Amino Acid Sequence (SEQ ID NO) |
|---|---|
| muCD37-3 | DIQMTQSPASLSVSVGETVTITCRASENIR |
| | SNLAWYQQKQGKSPQLLVNVATNLADGVPS |
| | RFSGSGSGTQYSLKINSLQSEDFGTYYCQH |
| | YWGTTWTFGGGTKLEIKR |
| | (SEQ ID NO: 72) |
| chCD37-3 | DIQMTQSPASLSVSVGETVTITCRASENIR |
| | SNLAWYQQKQGKSPQLLVNVATNLADGVPS |
| | RFSGSGSGTQYSLKINSLQSEDFGTYYCQH |
| | YWGTTWTFGGGTKLEIKR |
| | (SEQ ID NO: 73) |
| huCD37-3 | DIQMTQSPSSLSVSVGERVTITCRASENIR |
| (1.0 | SNLAWYQQKPGKSPKLLVNVATNLADGVPS |
| and 1.1) | RFSGSGSGTDYSLKINSLQPEDFGTYYCQH |
| | YWGTTWTFGQGTKLEIKR |
| | (SEQ ID NO: 74) |
| muCD37-12 | DIVLTQSPASLAVSLGQRATISCRASQSV |
| | STSSYSYLYWFQQKPGQPPKLLIKYASNL |
| | ASGVPARFSGSGSGTDFTLNIHPVEEEDT |
| | ATYYCQHSWEIPYTFGGGTKLEIKR |
| | (SEQ ID NO: 75) |
| chCD37-12 | DIVLTQSPASLAVSLGQRATISCRASQSV |
| | STSSYSYLYWFQQKFGQPPKLLIKYASNL |
| | ASGVPARFSGSGSGTDFTLNIHPVEEEDT |
| | ATYYCQHSWEIPYTFGGGTKLEIKR |
| | (SEQ ID NO: 76) |
| muCD37-38 | QIVLTQSPAIMSASPGEKVTMTCSASSSV |
| | TYMHWYQQKSGTSPKRWIYDTSKLASGVP |
| | ARFSGGGSGTSYSLTISSMEAEDAATYYC |
| | QQWISNPPTFGGGTKLEIKR |
| | (SEQ ID NO: 77) |
| chCD37-38 | QIVLTQSPAIMSASPGEKVTMTCSASSSV |
| | TYMHWYQQKSGTSPKRWIYDTSKLASGVP |
| | ARFSGGGSGTSYSLTISSMEAEDAATYYC |
| | QQWISNPPTFGGGTKLEIKR |
| | (SEQ ID NO: 78) |
| huCD37-38 | DIVLTQSPASMSASPGERVTMTCSASSSV |
| | TYMHWYQQKPGTSPKRWIYDTSKLASGVP |
| | ARFSGSGSGTSYSLTISSMEAEDAATYYC |
| | QQWISNFPTFGGGTKLEIKR |
| | (SEQ ID NO: 79) |
| muCD37-50 | QIVLTQSPAIMSASPGEKVTMTCSATSSV |
| | TYMHWYQQKSGTSPKRWIYDTSKLPYGVP |
| | GRFSGSGSGTSYSLTISSMEAEDAATYYC |
| | QQWSDNPPTFGSGTKLEIKR |
| | (SEQ ID NO: 80) |
| huCD37-50 | EIVLTQSPATMSASPGERVTMTCSATSSV |
| | TYMHWYQQKPGQSPKRWIYDTSNLPYGVP |
| | ARFSGSGSGTSYSLTISSMEAEDAATYYC |
| | QQWSDNPPTFGQGTKLEIKR |
| | (SEQ ID NO: 81) |
| muCD37-51 | QIVLTQSPAIMSASPGEKVTMTCSATSSV |
| | TYMHWYQQKSGTSPKRWIYDTSKLASGVP |
| | ARFSGSGSGTSYSLTISNMEAEDAATYYC |
| | QQWSSNPPTFGSGTKLEIKR |
| | (SEQ ID NO: 82) |
| huCD37-51 | EIVLTQSPATMSASPGERVTMTCSATSSV |
| | TYMHWYQQKPGQSPKRWIYDTSKLASGVP |
| | ARFSGSGSGTSYSLTISSMEAEDAATYYC |
| | QQWSSNPPTFGQGTKLEIKR |
| | (SEQ ID NO: 83) |
| muCD37-56 | QIVLTQSPAFMSASPGDKVTMTCSASSSV |
| | TYMHWYQQKSGTSPKRWIYDTSKLASGVP |
| | ARFSGGGSGTSYSLTISTMEAEDAATYYC |
| | QQWISDPFTFGGGTKLEIKR |
| | (SEQ ID NO: 84) |
| huCD37-56 | DIVLTQSPAFMSASPGEKVTMTCSASSSV |
| | TYMHWYQQKPDQSPKRWIYDTSNLASGVP |
| | SRFSGGGSGTDYSLTISSMEAEDAATYYC |
| | QQWISDPPTFGQGTKLEIKR |
| | (SEQ ID NO: 85) |
| muCD37-57 | QIVLTQSPAIMSASPGEKVTMTCSATSSV |
| | TYMHWYQQKSGTSPKRWIYDTSKLASGVP |
| | ARFSGSGSGTSYSLTISSMEAEDAATYYC |
| | QQWSDNPPTFGSGTKLEIKR |
| | (SEQ ID NO: 86) |
| huCD37-57 | EIVLTQSPATMSASPGERVTMTCSATSSV |
| | TYMHWYQQKPGQSPRRWIYDTSNLASGVP |
| | ARFSGSGSGTSYSLTISSMEAEDAATYYC |
| | QQWSDNPPTFGQGTKLEIKR |
| | (SEQ ID NO: 87) |

### Full-Length Heavy Chain Amino Acid Sequences

| | Full-Length Chain Amino |
|---|---|
| Antibody | Acid Sequence (SEQ ID NO) |
| muCD37-3 | QVQVKESGPGLVAPSQSLSITCTVSGFSL |
| | TTSGVSWVRQPPGKGLEWLGVIWGDGSTN |
| | YHSALKSRLSIKKDHSKSQVFLKLNSLQT |
| | DDTATYYCAKGGYSLAHWGQGTLVTVSAA |
| | KTTAPSVYPLAPVCGDTTGSSVTLGCLVK |
| | GYFPEPVTLTWNSGSLSSGVHTFPAVLQS |
| | DLYTLSSSVTVTSSTWPSQSITCNVAHPA |
| | SSTKVDKKIEPRGPTIKPCPPCKCPAPNL |
| | LGGPSVFIFPPKIKDVLMISLSPIVTCVV |
| | VDVSEDDPDVQISWFVNNVEVHTAQTQTH |
| | REDYNSTLRVVSALPIQHQDWMSGKEFKC |
| | KVNNKDLPAPIERTISKPKGSVRAPQVYV |
| | LPPPEEEMTKKQVTLTCMVTDFMPEDIYV |
| | EWYNNGKTELNYKNTEPVLDSDGSYFMYS |
| | KLRVEKKNWVERNSYSCSVVHEGLHNHHT |
| | TKSFSRTPGK (SEQ ID NO: 88) |
| chCD37-3 | QVQVKESGPGLVAPSQSLSITCTVSGFSL |
| | TTSGVSWVRQPPGKGLEWLGVIWGDGSTN |
| | YHSALKSRLSIKKDHSKSQVFLKLNSLQT |
| | DDTATYYCAKGGYSLAHWGQGTLVTVSAA |
| | STKGPSVFPLAPSSKSTSGGTAALGCLVK |
| | DYFPEPVTVSWNSGALTSGVHTFPAVLQS |
| | SGLYSLSSVVTVPSSSLGTQTYICNVNHK |
| | PSNTKVDKVEPKSCDKTHTCPPCPAPELL |
| | GGPSVFLFPPKPKDTLMISRTPEVTCVVV |
| | DVSHEDPEVKFNWYVDGVEVHNAKTKPRE |
| | EQYNSTYRVVSVLTVLHQDWLNGKEYKCK |
| | VSNKALPAPIEKTISKAKGQPREPQVYTL |
| | PPSRDELTKNQVSLTCLVKGFYPSDIAVE |
| | WESNGQPENNYKTTPPVLDSDGSFFLYSK |
| | LTVDKSRWQQGNVFSCSVMHEALHNHYTQ |
| | KSLSLSPGK (SEQ ID NO: 89) |
| huCD37-3v1.0 | QVQVQESGPGLVAPSQTLSITCTVSGFSL |
| | TTSGVSWVRQPPGKGLEWLGVIWGDGSTN |
| | YHPSLKSRLSIKKDHSKSQVFLKLNSLTA |
| | ADTATYYCAKGGYSLAHWGQGTLVTVSSA |
| | STKGPSVFPLAPSSKSTSGGTAALGCLVK |
| | DYFPEPVTVSWNSGALTSGVHTFPPAVLQ |
| | SSGLYSLSSVVTVPSSSLGTQTYICNVNH |
| | KPSNTKVDKKVEPKSCDKTHTCPPCPAPE |
| | LLGGPSVFLFPPKPKDTLMISRTPEVTCV |
| | VVDVSHEDPEVKFNWYVDGVEVHNAKTKF |
| | REEQYNSTYRVVSVLTVLHQDWLNGKEYK |
| | CKVSNKALPAPIEKTISKAKGQPREPQVY |
| | TLPPSRDELTKNQVSLTCLVKGFYPSDIA |
| | VEWESNGQPENNYKTTPPVLDSDGSFFLY |
| | SKLTVDKSRWQQGNVFSCSVMHEALHNHY |
| | TQKSLSLSPG (SEQ ID NO: 90) |
| huCD37-3v1.1 | QVQVQESGPGLVAPSQTLSITCTVSGFSL |
| | TTSGVSWVRQPPGKGLEWLGVIWGDGSTN |
| | YHSSLKSRLSIKKDHSKSQVFLKLNSLTA |
| | ADTATYYCAKGGYSLAHWGQGTLVTVSSA |
| | STKGPSVFPLAPSSKSTSGGTAALGCLVK |
| | DYFPEPVTVSWNSGALTSGVHTFPAVLQS |
| | SGLYSLSSVVTVPSSSLGTQTYICNVNHK |
| | PSNTKVDKKVEPKSCDKTHTCPPCPAPEL |
| | LGGPSVFLFPPKPKDTLMISRTPEVTCVV |
| | VDVSHEDPEVKFNWYVDGVEVHNAKTKPR |
| | EEQYNSTYRVVSVLTVLHQDWLNGKEYKC |
| | KVSNICALPAPIEKTISKAKGQPREPQVY |
| | TLPPSRDELTKNQVSLTCLVKGFYPSDIA |
| | VEWESNGQPENNYKTTPPVLDSDGSFFLY |
| | SKLTVDKSRWQQGNVFSCSVMHEALHNHY |
| | TQKSLSLSPG (SEQ ID NO: 91) |
| muCD37-12 | QIQLVQSGPELKKPGETVKISCKASGYTF |
| | TKYGMNWVKQAQGKGLKWMGWINTNTGES |
| | RNAEEFKGRFAFSLETSASTAYLQINNLK |
| | YEDTATYFCGRGTVVADWGQGTTLTVSSA |
| | KTTAPSVYPLAPVCGDTTGSSVTLGCLVK |
| | GYFPEPVTLTWNSGSLSSGVHTFPAVLQS |
| | DLYTLSSSVTVTSSTWPSQSITCNVAHPA |
| | SSTKVDKKIEPRGPTIKPCPPCKCPAPNL |
| | LGGPSVFIFPPKIKDVLMISLSPIVTCVV |
| | VDVSEDDPDVQISWFVNNVEVHTAQTQTH |
| | REDYNSTLRVVSALPIQHQDWMSGKEFKC |
| | KVNNKDLPAPIERTISKPKGSVRAPQVYV |
| | LPPPEEEMTKKQVTLTCMVTDFMPEDIYV |
| | EWTNNGKTELNYKNTEPVLDSDGSYFMYS |
| | KLRVEKKNWVERNSYSCSVVHEGLHNHHT |
| | TKSFSRTPGK (SEQ ID NO: 92) |
| chCD37-12 | QIQLVQSGPELKKPGETVKISCKASGYTF |
| | TKYGMNWVKQAQGKGLKWMGWINTNTGES |
| | RNAEEFKGRFAFSLETSASTAYLQINNLK |
| | YEDTATYFCGRGTVVADWGQGTTLTVSSA |
| | STKGPSVFPLAPSSKSTSGGTAALGCLVK |
| | DYFPEPVTVSWNSGALTSGVHTFPAVLQS |
| | SGLYSLSSVVTVPSSSLGTQTYICNVNHK |
| | PSNTKVDKKVEPKSCDKTHTCPPCPAPEL |
| | LGGPSVFLFPPKPKDTLMISRTPEVTCVV |
| | VDVSHEDPEVKFNWYVDGVEVHNAKTKPR |
| | EEQYNSTYRVVSVLTVLHQDWLNGKEYKC |
| | KVSNKALPAPIEKTISKAKGQPREPQVYT |
| | LPPSRDELTKNQVSLTCLVKGFYPSDIAV |
| | EWESNGQPENNYKTTPPVLDSDGSFFLYS |
| | KLTVDKSRWQQGNVFSCSVMHEALHNHYT |
| | QKSLSLSPGK (SEQ ID NO: 93) |
| muCD37-38 | DVQLQESGPDLVKPSQSLSLTCTVTGYSI |
| | TSGFGWHWIRQFPGNKLEWMAYILYSGGT |
| | DYNPSLKSRISITRDTSKNQFFLRLSSVT |
| | TEDTATYYCARGYYGYGAWFVYWGQGTLV |
| | TVSAAKTTPPSVYPLAPGSAAQTNSMVTL |
| | GCLVKGYFPEPVTVTWNSGSLSSGVHTFP |
| | AVLESDLYTLSSSVTVPSSMRPSETVTCN |
| | VAHPASSTKVDKKIVPRDCGCKPCICTVP |
| | EVSSVFIFPPKPKDVLTITLTPKVTCVVV |
| | DISKDDPEVQFSWFVDDVEVHTAQTQPRE |
| | EQFNSTFRSVSELPIMHQDWLNGKEFKCR |
| | VNSAAFPAPIEKTISKTKGRPKAPQVYTI |
| | PPPKEQMAKDKVSLTCMITDFFPEDITVE |
| | WQWNGQPAENYKNTQPIMNTNGSYFVYSK |
| | LNVQKSNWEAGNIFTCSVLHEGLHNHHTE |
| | KSLSIISPGK (SEQ ID NO: 94) |
| chCD37-38 | QVQLQESGPDLVKPSQSLSLTCTVTGYSI |
| | TSGFGWHWIRQFPGNKLEWMAYILYSGGT |
| | DYNPSLKSRISITRDTSKNQFFLRLSSVT |
| | TEDTATYYCARGYYGYGAWFVYWGQGTLV |
| | TVSAASTKGPSVFPLAPSSKSTSGGTAAL |
| | GCLVKDYFPEPVTVSWNSGALTSGVHTFP |
| | AVLQSSGLYSLSSVVTVPSSSLGTQTYIC |
| | NVNHKPSNTKVDKKVEPKSCDKTHTCPPC |
| | PAPELLGGPSVFLFPPKPKDTLMISRTPE |
| | VTCVVVDVSHEDPEVKFNWYVDGVEVHNA |
| | KTKPREEQYNSTYRVVSVLTVLHQDWLNG |
| | KEYKCKVSNKALPAPIEKTISKAKGQPRE |
| | PQVYTLPPSRDELTKNQVSLTCLVKGFYP |
| | SDIAVEWESNGQPENNYKTTPPVLDSDGS |
| | FFLYSKLTVDKSRWQQGNVFSCSVMHEAL |
| | HNHYTQKSLSLSPG (SEQ ID NO: 95) |
| huCD37-38 | QVQLQESGPGLVKPSQSLSLTCTVSGYSI |
| | TSGFGWHWIRQFPGKGLEWMAYILYSGGT |
| | DYNPSLKSRISITRDTSKNQFFLRLSSVT |
| | AADTATYYCARGYYGYGAWFVYWGQGTLV |
| | TVSSASTKGPSVFPLAPSSKSTSGGTAAL |
| | GCLVKDYFPEPVTVSWNSGALTSGVHTFP |
| | AVLQSSGLYSLSSVVTVPSSSLGTQTYIC |
| | NVNHKPSNTKVDKKVEPKSCDKTHTCPPC |
| | PAPELLGGPSVFLFPPKPKDTLMISRTPE |
| | VTCVVVDVSHEDPEVKFNWYVDGVEVHNA |
| | KTKPREEQYNSTYRVVSVLTVLHQDWLNG |
| | KEYKCKVSNKALPAPIEKTISKAKGQPRE |
| | PQVYTLPPSRDELTKNQVSLTCLVKGFYP |
| | SDIAVEWESNGQPENNYKTTPPVLDSDGS |
| | FFLYSKLTVDKSRWQQGNVFSCSVMHEAL |
| | HNHYTQKSLSLSPG (SEQ ID NO: 96) |
| muCD37-50 | DVQLQESGPDLLKPSQSLSLTCTVTGYSI |
| | TSGFAWHWIRQFPGNKLEWMGYILYSGST |
| | VYSPSLKSRISITRDTSKNHFFLQLNSVT |
| | TEDTATYYCARGYYGYGAWFAYWGQGTLV |
| | TVSAAKTTAPSVYPLAPVCGDTTGSSVTL |
| | GCLVKGYFPEPVTLTWNSGSLSSGVHTFP |
| | AVLQSDLYTLSSSVTVTSSTWPSQSITCN |
| | VAHPASSTKVDKKIEPRGPTIKPCPPCKC |
| | PAPNLLGGPSVFIFPPKIKDVLMISLSPI |
| | VTCVVVDVSEDDPDVQISWFVNNVEVHTA |
| | QTQTHREDYNSTLRVVSALPIQHQDWMSG |
| | KEFKCKVNNKDLPAPIERTISKPKGSVRA |
| | PQVYVLPPPEEEMTKKQVTLTCMVTDFMP |
| | EDIYVEWTNNGKTELNYKNTEPVLDSDGS |
| | YFMYSKLRVEKKNWVERNSYSCSVVHEGL |
| | HNHHTTKSFSRTPGK (SEQ ID NO: 97) |
| huCD37-50 | QVQLQESGPGLLKPSQSLSLTCTVSGYSI |
| | TSGFAWHWIRQHPGNKLEWMGYILYSGST |
| | VYSPSLKSRISITRDTSKNHFFLQLNSVT |
| | AADTATYYCARGYYGYGAWFAYWGQGTLV |
| | TVSAASTKGPSVFPLAPSSKSTSGGTAAL |
| | GCLVKDYFPEPVTVSWNSGALTSGVHTFP |
| | AVLQSSGLYSLSSVVTVPSSSLGTQTYIC |
| | NVNHKPSNTKVDKKVEPKSCDKTHTCPPC |
| | PAPELLGGPSVFLFPPKPKDTLMISRTPE |
| | VTCVVVDVSHEDPEVKFNWYVDGVEVHNA |
| | KTKPREEQYNSTYRVVSVLTVLHQDWLNG |
| | KEYKCKVSNKALPAPIEKTISKAKGQPRE |
| | PQVYTLPPSRDELTKNQVSLTCLVKGFYP |
| | SDIAVEWESNGQPENNYKTTPPVLDSDGS |
| | FFLYSKLTVDKSRWQQGNVFSCSVMHEAL |
| | HNHYTQKSLSLSPG (SEQ ID NO: 98) |
| muCD37-51 | DVQLQESGPDLLKPSQSLSLTCTVTGYSI |
| | SSGFAWHWIRQFPGNKLEWMGYIHYSGST |
| | NYSPSLKSRISITRDSSKNQFFLQLNSVT |
| | TEDTATYYCARGYYGFGAWFVYWGQGTLV |
| | TVSAAKTTAPSVYPLAPVCGDTTGSSVTL |
| | GCLVKGYFPEPVTLTWNSGSLSSGVHTFP |
| | AVLQSDLYTLSSSVTVTSSTWPSQSITCN |
| | VAHPASSTKVDKKIEPRGPTIKPCPPCKC |
| | PAPNLLGGPSVFIFPPKIKDVLMISLSPI |
| | VTCVVVDVSEDDPDVQISWFVNNVEVHTA |
| | QTQTHREDYNSTLRVVSALPIQHQDWMSG |
| | KEFKCKVNNKDLPAPIERTISKPKGSVRA |
| | PQVYVLPPPEEEMTKKQVTLTCMVTDFMP |
| | EDIYVEWTNNGKTELNYKNTEFVLDSDGS |
| | YFMYSKLRVEKKNWVERNSYSCSVVHEGL |
| | HNHHTTKSFSRTPGK (SEQ ID NO: 99) |
| huCD37-51 | EVQLVESGPEVLKPGESLSLTCTVSGYSI |
| | SSGFAWHWIRQFPGKGLEWMGYIHYSGST |
| | NYSPSLQGRISITRDSSINQFFLQLNSVT |
| | ASDTATYYCARGYYGFGAWFVYWGQGTLV |
| | TVSAASTKGPSVFPLAPSSKSTSGGTAAL |
| | GCLVKDYFPEPVTVSWNSGALTSGVHTFP |
| | AVLQSSGLYSLSSVVTVPSSSLGTQTYIC |
| | NVNHKPSNTKVDKKVEPKSCDKTHTCPPC |
| | PAPELLGGPSVFLFPPKPDTLMISRTPEV |
| | TCVVVDVSHEDPEVKFNWYVDGVEVHNAK |
| | TKPREEQYNSTYRVVSVLTVLHQDWLNGK |
| | EYKCKVSNKALPAPIEKTISKAKGQPREP |
| | QVYTLPPSRDELTKNQVSLTCLVKGFYPS |
| | DIAVEWESNGQPENNYKTTPPVLDSDGSF |
| | FLYSKLTVDKSRWQQGNVFSCSVMHEALH |
| | NHYTQKSLSLSPG (SEQ ID NO: 100) |
| muCD37-56 | DVQLQESGPDLVKPSQSLSLTCTVTGYSI |
| | TSGFAWHWIRQFPGNKLEWMGYIHYSGGT |
| | NYNPSLKSRVSITRDTSKNQFFLQLNSVT |
| | TEDTATYYCARGYYGFGAWFAYWGQGTLV |
| | PVSAAKTTPPSVYPLAPGSAAQTNSMVTL |
| | GCLVKGYFPEPVTVTWNSGSLSSGVHTFP |
| | AVLESDLYTLSSSVTVPSSMRPSETVTCN |
| | VAHPASSTKVDKKIVPRDCGCKPCICTVP |
| | EVSSVFIFPPKPKDVLTITLTPKVTCVVV |
| | DISKDDPEVQFSWFVDDVEVHTAQTQPRE |
| | EQFNSTFRSVSELPIMHQDWLNGKEFKCR |
| | VNSAAFPAPIEKTISKTKGRPKAPQVYTI |
| | PPPKEQMAKDKVSLTCMITDFFPEDITVE |
| | WQWNGQPAENYKNTQPIMNTNGSYFVYSK |
| | LNVQKSNWEAGNTFTCSVLHEGLHNHHTE |
| | KSLSHSPGK (SEQ ID NO: 101) |
| huCD37-56 | QVQLQESGPGLVKPSQSLSLTCTVSGYSI |
| | TSGFAWHWIRQFPGKGLEWMGYIHYSGGT |
| | NYNPSLKSRVSITRDTSKNQFFLQLNSVT |
| | AADTATYYCARGYYGFGAWFAYWGQGTLV |
| | PVSAASTKGPSVFPLAPSSKSTSGGTAAL |
| | GCLVKDYFPEPVTVSWNSGALTSGVHTFP |
| | AVLQSSGLYSLSSVVTVPSSSLGTQTYIC |
| | NVNHKPSNTKVDKKVEPKSCDKTHTCPPC |
| | PAPELLGGPSVFLFPPKPKDTLMISRTPE |
| | VTCVVVDVSHEDPEVKFNWYVDGVEVHNA |
| | KTKPREEQYNSTYRVVSVLTVLHQDWLNG |
| | KEYKCKVSNKALPAPIEKTISKAKGQPRE |
| | PQVYTLPPSRDELTKNQVSLTCLVKGFYP |
| | SDIAVEWESNGQPENNYKTTPPVLDSDGS |
| | FFLYSKLTVDKSRWQQGNVFSCSVMHEAL |
| | HNHYTQKSLSLSPG (SEQ ID NO: 102) |
| muCD37-57 | DVQLQESGPDLLKPSQSLSLTCTVTGYSI |
| | TSGFAWHWIRQFPGNKLEWMGYILYSGST |
| | VYSPSLKSRISITRDTSKNQFFLQLNSVT |
| | TEDTATYYCARGYYGYGAWFAYWGQGTLV |
| | TVSAAKTTAPSVYPLAPVCGDTTGSSVTL |
| | GCLVKGYFPEPVTLTWNSGSLSSGVHTFP |
| | AVLQSDLYTLSSSVTVTSSTWPSQSITCN |
| | VAHPASSTKVDKKIEPRGPTIKPCPPCKC |
| | PAPNLLGGPSVFIFPPKIKDVLMISLSPI |
| | VTCVVVDVSEDDPDVQISWFVNNVEVHTA |
| | QTQTHREDYNSTLRVVSALPIQHQDWMSG |
| | KEFKCKVNNKDLPAPIERTISKPKGSVRA |
| | PQVYVLPPPEEEMTKKQVTLTCMVTDFMP |
| | EDIYVEWTNNGKTELNYKNTEPVLDSDGS |
| | YFMYSKLRVEKKNWVERNSYSCSVVHEGL |
| | HNHHTTKSFSRTPGK (SEQ ID NO: 103) |
| huCD37-57 | QVQLQESGPGLLKPSQSLSLTCTVSGYSI |
| | TSGFAWHWIRQFPGKGLEWMGYILYSGST |
| | VYSPSLKSRISITIRDTSKNQFFLQLNSV |
| | TAADTATYYCARGYYGYGAWFAYWGQGTL |
| | VTVSAASTKGPSVFPLAPSSKSTSGGTAA |
| | LGCLVKDYFPEPVTVSWNSGALTSGVHTF |
| | PAVLQSSGLYSLSSVVTVPSSSLGTQTYI |
| | CNVNHKPSNTKVDKKVEPKSCDKTHTCPP |
| | CPAPELLGGPSVFLFPPKPKDTLMISRTP |
| | EVTCVVVDVSHEDPEVKFNWYVDGVEVHN |
| | AKTKPREEQYNSTYRVVSVLTVLHQDWLN |
| | GKEYKCKVSNKALPAPIEKTISKAKGQPR |
| | EPQVYTLPPSRDELTKNQVSLTCLVKGFY |
| | PSDIAVEWESNGQPENNYKTTPPVLDSDG |
| | SFFLYSKLTVDKSRWQQGNVFSCSVMHEA |
| | LHNHYTQKSLSLSPG (SEQ ID NO: 104) |

### Full-Length Light Chain Amino Acid Sequences

| | Full-length Light Chain Amino |
|---|---|
| Antibody | Acid Sequence (SEQ ID NO) |
| muCD37-3 | DIQMTQSPASLSVSVGETVTITCRASENI |
| | RSNLAWYQQKQGKSPQLLVNVATNLADG |
| | VPSRFSGSGSGTQYSLKINSLQSEDFGT |
| | YYCQHYWGTTWTFGGGTKLEIKRADAAP |
| | TVSIFPPSSEQLTSGGASVVCFLNNFYP |
| | KDINVKWKIDGSERQNGVLNSWTDQDSK |
| | DSTYSMSSTLTLTKDEYERHNSYTCEAT |
| | HKTSTSPIVKSFNRNEC |
| | (SEQ ID NO: 105) |
| chCD37-3 | DIQMTQSPASLSVSVGETVTITCRASENI |
| | RSNLAWYQQKQGKSPQLLVNVATNLADG |
| | VPSRFSGSGSGTQYSLKINSLQSEDFGT |
| | YYCQHYWGTTWTFGGGTKLEIKRTVAAP |
| | SVFIFPPSDEQLKSGTASVVCLLNNFYP |
| | REAKVQWKVDNALQSGNSQESVTEQDSK |
| | DSTYSLSSTLTLSKADYEKHKVYACEVT |
| | HQGLSSPVTKSFNRGEC |
| | (SEQ ID NO: 106) |
| huCD37-3 | DIQMTQSPSSLSVSVGERVTITCRASENI |
| (1.0 and 1.1) | RSNLAWYQQKPGKSPKLLVNVATN |
| | LADGVPSRFSGSGSGTDYSLKINSLQPE |
| | DFGTYYCQHYWGTTWTFGQGTKLEIKRT |
| | VAAPSVFIFPPSDEQLKSGTASVVCLLN |
| | NFYPREAKVQWKVDNALQSGNSQESVTE |
| | QDSKDSTYSLSSTLTLSKADYEKHKVYA |
| | CEVTHQGLSSPVTKSFNRGEC |
| | (SEQ ID NO: 107) |
| muCD37-12 | DIVLTQSPASLAVSLGQRATISCRASQS |
| | VSTSSYSYLYWFQQKPGQPPKLLIKYAS |
| | NLASGVPARFSGSGSGTDFTLNIHPVEE |
| | EDTATYYCQHSWEIPYTFGGGTKLEIKR |
| | ADAAPTVSIFPPSSEQLTSGGASVVCFL |
| | NNFYPKDINVKWKIDGSERQNGVLNSWT |
| | DQDSKDSTYSMSSTLTLTKDEYERHNSY |
| | TCEATHKTSTSPIVKSFNRNEC |
| | (SEQ ID NO: 108) |
| chCD37-12 | DIVLTQSPASLAVSLGQRATISCRASQS |
| | VSTSSYSYLYWFQQKPGQPPKLLIKYAS |
| | NLASGVPARFSGSGSGTDFTLNIHPVEE |
| | EDTATYYCQHSWEIPYTFGGGTKLEIKR |
| | TVAAPSVFIFPPSDEQLKSGTASVVCLL |
| | NNFYPREAKVQWKVDNALQSGNSQESVT |
| | EQDSKDSTYSLSSTLTLSKADYEKHKVY |
| | ACEVTHQGLSSPVTKSFNRGEC |
| | (SEQ ID NO: 109) |
| muCD37-38 | QIVLTQSPAIMSASPGEKVTMTCSASSS |
| | VTYMHWYQQKSGTSPKRWIYDTSKLASG |
| | VPARFSGGGSGTSYSLTISSMEAEDAAT |
| | YYCQQWISNPPTFGGGTKLEIKRADAA |
| | PTVSIFPPSSEQLTSGGASVVCFLNNFY |
| | PKDINVKWKIDGSERQNGVLNSWTDQDS |
| | KDSTYSMSSTLTLTKDEYERHNSYTCEA |
| | THKTSTSPIVKSFNRNEC |
| | (SEQ ID NO: 110) |
| chCD37-38 | QIVLTQSPAIMSASPGEKVTMTCSASSS |
| | VTYMHWYQQKSGTSPKRWIYDTSKLASG |
| | VPARFSGGGSGTSYSLTISSMEAEDAAT |
| | YYCQQWISNPPTFGGGTKLEIKRTVAAP |
| | SVFIFPPSDEQLKSGTASVVCLLNNFYP |
| | REAKVQWKVDNALQSGNSQESVTEQDSK |
| | DSTYSLSSTLTLSKADYEKHKVYACEVT |
| | HQGLSSPVTKFNRGEC |
| | (SEQ ID NO: 111) |
| huCD37-38 | DIVLTQSPASMSASPGERVTMTCSASSS |
| | VTYMHWYQQKPGTSPKRWIYDTSKLASG |
| | VPARFSGSGSGTSYSLTISSMEAEDAAT |
| | YYCQQWISNPPTFGGGTKLEIKRTVAAP |
| | SVFIFPPSDEQLKSGTASVVCLLNNFYP |
| | REAKVQWKVDNALQSGNSQESVTEQDSK |
| | DSTYSLSSTLTLSKADYEKHKVYACEVT |
| | HQGLSSPVTKSFNRGEC |
| | (SEQ ID NO: 112) |
| muCD37-50 | QIVLTQSPAIMSASPGEKVTMTCSATSS |
| | VTYMHWYQQKSGTSPKRWIYDTSKLPYG |
| | VPGRFSGSGSGTSYSLTISSMEAEDAAT |
| | YYCQQWSDNPPTFGSGTKLEIKRADAAP |
| | TVSIFPPSSEQLTSGGASVVCFLNNFYP |
| | KDINVKWKIDGSERQNGVLNSWTDQDSK |
| | DSTYSMSSTLTLTKDEYERHNSYTCEAT |
| | HKTSTSPIVKSFNRNEC |
| | (SEQ ID NO: 113) |
| huCD37-50 | EIVLTQSPATMSASPGERVTMTCSATSS |
| | VTYMHWYQQKPGQSPKRWIYDTSNLPYG |
| | VPARFSGSGSGTSYSLTISSMEAEDAAT |
| | YYCQQWSDNPPTFGQGTKLEIKRTVAAP |
| | SVFIFPPSDEQLKSGTASVVCLLNNFYP |
| | REAKVQWKVDNALQSGNSQESVTEQDSK |
| | DSTYSLSSTLTLSKADYEKHKVYACEVT |
| | HQGLSSPVTKSFNRGEC |
| | (SEQ ID NO: 114) |
| muCD37-51 | QIVLTQSPAIMSASPGEKVTMTCSATSS |
| | VTYMHWYQQKSGTSPKRWIYDTSKLASG |
| | VPARFSGSGSGTSYSLTISNMEAEDAAT |
| | YYCQQWSSNPPTFGSGTKLEIKRADAAP |
| | TVSIFPPSSEQLTSGGASVVCFLNNFYP |
| | KDINVKWKIDGSERQNGVLNSWTDQDSK |
| | DSTYSMSSTLTLTKDEYERHNSYTCEAT |
| | HKTSTSPIVKSFNRNEC |
| | (SEQ ID NO: 115) |
| huCD37-51 | EIVLTQSPATMSASPGERVTMTCSATSS |
| | VTYMHWYQQKPGQSPKRWIYDTSKLASG |
| | VPARFSGSGSGTSYSLTISSMEAEDAAT |
| | YYCQQWSSNPPTFGQGTKLEIKRTVAAP |
| | SVFIFPPSDEQLKSGTASVVCLLNNFYP |
| | REAKVQWKVDNALQSGNSQESVTEQDSK |
| | DSTYSLSSTLTLSKADYEKHKVYACEVT |
| | HQGLSSPVTKSFNRGEC |
| | (SEQ ID NO: 116) |
| muCD37-56 | QIVLTQSPAFMSASPGDKVTMTCSASSS |
| | VTYMHWYQQKSGTSPKRWIYDTSKLASG |
| | VPARFSGGGSGTSYSLTISTMEAEDAAT |
| | YYCQQWISDPPTFGGGTKLEIKRADAAP |
| | TVSIFPPSSEQLTSGGASVVCFLNNFYP |
| | KDINVKWKIDGSERQNGVLNSWTDQDSK |
| | DSTYSMSSTLTLTKDEYERHNSYTCEAT |
| | HKTSTSPIVKSFNRNEC |
| | (SEQ ID NO: 117) |
| huCD37-56 | DIVLTQSPAFMSASPGEKVTMTCSASSS |
| | VTYMHWYQQKPDQSPKRWIYDTSNLASG |
| | VPSRFSGGGSGTDYSLTISSMEAEDAAT |
| | YYCQQWISDPPTFGQGTKLEIKRTVAAP |
| | SVFIFPPSDEQLKSGTASVVCLLNNFYP |
| | REAKVQWKVDNALQSGNSQESVTEQDSK |
| | DSTYSLSSTLTLSKADYEKHKVYACEVT |
| | HQGLSSPVTKSFNRGEC |
| | (SEQ ID NO: 118) |
| muCD37-57 | QIVLTQSPAIMSASPGEKVTMTCSATSS |
| | VTYMHWYQQKSGTSPKRWIYDTSKLASG |
| | VPARFSGSGSGTSYSLTISSMEAEDAAT |
| | YYCQQWSDNPPTFGSGTKLEIKRADAAP |
| | TVSIFPPSSEQLTSGGASVVCFLNNFYP |
| | KDINVKWKIDGSERQNGVLNSWTDQDSK |
| | DSTYSMSSTLTLTKDEYERHNSYTCEAT |
| | HKTSTSPIVKSFNRNEC |
| | (SEQ ID NO: 119) |
| huCD37-57 | EIVLTQSPATMSASPGERVTMTCSATSS |
| | VTYMHWYQQKPGQSPRRWIYDTSNLASG |
| | VPARFSGSGSGTSYSLTISSMEAEDAAT |
| | YYCQQWSDNPPTFGQGTKLEIKRTVAAP |
| | SVFIFPPSDEQLKSGTASVVCLLNNFYP |
| | REAKVQWKVDNALQSGNSQESVTEQDSK |
| | DSTYSLSSTLTLSKADYEKHKVYACEVT |
| | HQGLSSPVTKSFNRGEC |
| | (SEQ ID NO: 120) |

### Variable Heavy Chain Polynucleotide Sequences

| | VH Polynucleotide |
|---|---|
| Antibody | Sequence (SEQ ID NO) |
| muCD37-3 | caggtgcaggtgaaggagtcaggacctgg |
| | cctggtggcgccctcacagagcctgtcc |
| | attacatgcactgtctcagggttctcat |
| | taaccacctctggtgtaagctgggttcg |
| | ccagcctccaggaaagggtctggagtgg |
| | ctgggagtaatatggggtgacgggagca |
| | caaactatcattcagactcaaatccaga |
| | ctgagcatcaagaaggatcactccaaga |
| | gccaagttttctcttaaaactgaacagt |
| | ctgcaaactgatgacacagccacgtact |
| | actgtgccaaaggaggctactcgttggc |
| | tcactggggccaagggactctggtcaca |
| | gtctagca |
| | (SEQ ID NO: 121) |
| chCD37-3 | aagcttgccaccatggctgtcctggcact |
| | gctcctctgcctggtgacatacccaagc |
| | tgtgtcctatcacaggtgcaggtgaagg |
| | agtcaggacctggcctggtggcgccctc |
| | acagagcctgtccattacatgcactgtc |
| | tcagggttctcattaaccacctctggtg |
| | taagctgggttcgccagcctccaggaaa |
| | gggtctggagtggctgggagtaatatgg |
| | ggtgacgggagcacaaactatcattcag |
| | ctctcaaatccagactgagcatcaagaa |
| | ggatcactccaagagccaagttttctta |
| | aaactgaacagtctgcaaactgatgaca |
| | cagccacgtactactgtgccaaaggagg |
| | ctactcgttggctcactggggccaaggg |
| | actctggtcacagtctctgcagcctcta |
| | cgaagggccc |
| | (SEQ ID NO: 122) |
| huCD37-3v1.0 | aagcttgccaccatgggttggagct |
| | gcattattctgtttctggtggccaccgc |
| | caccggtgtgcactcacaagtccaagtc |
| | caagaatctggtccaggtctggtggccc |
| | cttcccaaactctgagcatcacctgtac |
| | cgtttctggttttagccttaccacctct |
| | ggtgtgagttgggtacgccaaccacccg |
| | gtaagggtctcgaatggctgggtgtaat |
| | ctggggtgatggttccacaaattaccat |
| | ccttccctcaagtcccgccttagcatca |
| | aaaaggatcacagcaaaagtcaagtttt |
| | acctgaaactgaatagtctgacgcagcc |
| | gatacagccacctactattgcgccaagg |
| | gtggttatagtcttgcacactggggtca |
| | aggtaccctcgttaccgtctcctcagct |
| | agtaccaagggccc |
| | (SEQ ID NO: 123) |
| huCD37-3v1.1 | aagcttgccaccatgggctggagct |
| | gtatcattctgtttctggtggcgacagc |
| | tactggggtccactcccaagtgcaggta |
| | caagagtccgggcctggattggtcgcac |
| | caagccagaccctctctatcacttgtac |
| | cgttagcgggttctctctgacaaccagt |
| | ggagtgagttgggtgaggcagccaccag |
| | gaaagggactggagtggctgggggtgat |
| | ttggggcgacggcagcacaaactatcat |
| | tccagtcttaaatctcggttgtccatta |
| | aaaaagaccatagtaaatctcaagtttt |
| | cctgaaactcaatagccgacagccgcag |
| | acactgctacgtattactgcgccaaagg |
| | aggatacagtctggctcactggggacag |
| | gggaccctggtgaccgtgtcatccgcat |
| | caacaaagggccc |
| | (SEQ ID NO: 124) |
| muCD37-12 | cagatccagttggtgcagtctggacctg |
| | agctgaagaagcctggagagacagtcaa |
| | gatacctgcaaggcttctgggtatacct |
| | tcacaaagtatggaatgaactgggtgaa |
| | gcaggctcaaggaaagggtttaaagtgg |
| | atgggctggataaacaccaacactggag |
| | agtcaagaaatgctgaagaattcaaggg |
| | acggtttgccttctctttggaaacctct |
| | gccagcactgcctatttgcagatcaaca |
| | acctcaaatatgaggacacggctacata |
| | tttctgtggaaggggcacggtagtagcg |
| | gactggggccaaggcaccactctcacag |
| | tctcctca |
| | (SEQ ID NO: 125) |
| chCD37-12 | aagcttgccaccatggggtggtcatgca |
| | taatcctctttctggtcgctactgctac |
| | cggtgtgcactcacagattcagctggtt |
| | caaagtggcccagagctgaaaaagccag |
| | gggaaacagtgaaaataagttgcaaggc |
| | atccggttacactttcacaaagtacggc |
| | atgaactgggtcaagcaggcccagggca |
| | aggggctcaaatggatgggttggatcaa |
| | taccaacactggcgagtctaggaatgct |
| | gaggagtttaagggccggtttgccttca |
| | gcctggagacaagtgccagcacagatac |
| | ctgcaaatcaacaatctgaagtatgagg |
| | atacagcaacctatttctgcggccgcgg |
| | cactgtcgttgcagactggggacaaggt |
| | accaccttgactgtatccagtgccagca |
| | ctaagggccc |
| | (SEQ ID NO: 126) |
| muCD37-38 | gatgtgcagcttcaggagtcaggacctg |
| | acctggtgaaaccttctcagtcactttc |
| | actcacctgcactgtcactggctactcc |
| | atcaccagtggttttggctggcactgga |
| | tccggcagtttccaggaaacaagaggaa |
| | tggatggcctacatactctacagtggtg |
| | gcactgactacaacccatctctcaaaag |
| | tcgaatctctatcactcgagacacttcc |
| | aagaaccagttcttctgcggttgagttc |
| | tgtgactactgaggacacagccacatat |
| | tactgtgcaagaggctactatggttacg |
| | gggcctggtttgtttactggggccaagg |
| | gactctggtcactgtctctgca |
| | (SEQ ID NO: 127) |
| chCD37-38 | aagcttccaccatgggctggagttgtat |
| | ctattctgtttttggtggccaccgccac |
| | tggagtccattcccaagtgcaactccag |
| | gaatctggccctgacctggttaagccat |
| | ctcagagcctctccctgacctgcactgt |
| | tacaggatactcaatcacatcaggcttt |
| | ggctggcactggatcagacaatttcccg |
| | ggaacaagttggaatggatggcttacat |
| | tctgtatagcgggggtaccgattacaat |
| | ccttccctcaagagccgaatctctatca |
| | ccagggatacaagcaagaaccaattttt |
| | tctccgcctcagctctgtgactaccgaa |
| | gataccgctacttactattgtgccaggg |
| | gctactatggatatggtgcatggttcgt |
| | ctattggggccagggaaccctggtgact |
| | gtgagcgctgcctctaccaagggccc |
| | (SEQ ID NO: 128) |
| huCD37-38 | aagcttgccaccatgggttggagctgca |
| | tcattcttttcctggtcgcactgcaact |
| | ggagtccactcacaggtccagctgcaag |
| | agtccggtcctgggcttgtgaaacccag |
| | ccagtccctcagtctcacctgtactgtc |
| | tctggctactctattaccagtgggttcg |
| | gctggcattggattaggcagtttcccgg |
| | taaggggctggagtggatggcatatatc |
| | ctgtacagcggaggaaccgattacaacc |
| | caagtctgaagagcaggatcagcattac |
| | ccgggacacaagcaaaaaccagtttttc |
| | cttcggctgtctagtgttacagctgcag |
| | acaccgctacttactattgtgctcgggg |
| | ttactatggctatggggcttggtttgtg |
| | tattggggacaaggcactcttgtgaccg |
| | tgagcagcgcctcaacaaagggccc |
| | (SEQ ID NO: 129) |
| muCD37-50 | gatgtgcagcttcaggagtcaggacctg |
| | acctgttgaaaccttctcagtcactttc |
| | actcacctgcactgtcactggctactcc |
| | atcaccagtggttttgcctggcactgga |
| | tccggcagtttccaggaaacaaactgga |
| | atggatgggctacatactctacagtggt |
| | agcactgtctacagcccatctctcaaaa |
| | gtcgaatctctatcactcgagacacatc |
| | caagaaccacttcttcctgcagttgaat |
| | tctgtgactactgaggacacagccacat |
| | attactgtgcaagagggtactatggtta |
| | cggcgcctggtttgcttactggggccaa |
| | gggactctggtcactgtctctgca |
| | (SEQ ID NO: 130) |
| huCD37-50 | aagcttgccaccatggggtggtcctgca |
| | taatcctttcctggttgctactgctacc |
| | ggagtccattcacaggtgcagctgcagg |
| | agtccggccccggcctgctcaagccttc |
| | tcagagtctgagtctgacttgtactgtt |
| | tctggctacagcataaccagcggtttcg |
| | cttggcactggatcagacagcatcccgg |
| | caacaaactggagtggatgggatacata |
| | ctgtactcaggctcaactgtctattccc |
| | cctgaaatcccggatcagtattacccgt |
| | gacacttctaagaaccatttttttctgc |
| | agctgaacagcgttaccgcagctgacac |
| | tgcaacctactactgtgcccggggatat |
| | tatggatacggagcttggttcgcttact |
| | ggggccaaggcaccctcgtaactgtgag |
| | tgctgcttccaccaagggccc |
| | (SEQ ID NO: 2) |
| muCD37-51 | gatgtgcagcttcaggagtcaggacctg |
| | acctgttgaaaccttctcagtcactttc |
| | actcacctgcactgtcactggctactcc |
| | atctccagtggttttgcctggcactgga |
| | tccggcagtttccaggaaacaaactgga |
| | atggatgggctacatacactacagtggt |
| | agcactaactacagcccatctctcaaaa |
| | gtcgaatctctatcactcgagactcatc |
| | caagaaccagttcttcctgcagttgaat |
| | tctgtgactactgaggacacagccacat |
| | attactgtgcaagaggatactatggttt |
| | cggcgcctggtttgtttactggggccaa |
| | gggactctggtcactgtctctgca |
| | (SEQ ID NO: 131) |
| huCD37-51 | Aagcttgccaccatgggttggtcttgca |
| | tcatcctgttcctggtggccactgccac |
| | tggcgtgcattcagaagttcagttggtg |
| | gagtccggcccagaagtgctgaaacccg |
| | gcgaatcactgtccctgacttgtaccgt |
| | gtcaggttatagcatcagcagcggcttt |
| | gcttggcactggattcggcagtttccag |
| | gcaagggactggaatggatgggctacat |
| | ccattacagtggctcaaccaattacagc |
| | cctagcctgcagggccgaatctctatta |
| | ccagggatagttctattaaccagttttt |
| | cctgcagcttaattccgtgactgcctct |
| | gacacagcaacttactattgcgcccgtg |
| | gctactacgggttcggagcctggtttgt |
| | atactggggtcagggcaccctggtcact |
| | gtctcagccgcctctaccaagggccc |
| | (SEQ ID NO: 3) |
| muCD37-56 | gatgtgcagcttcaggagtcaggacctg |
| | acctggtgaaaccttctcagtcactttc |
| | actcacctgcactgtcactggctactcc |
| | atcaccagtggttttgcctggcactgga |
| | tccggcagtttccaggaaacaaactgga |
| | atggatgggctacatacactacagtggt |
| | ggcactaactacaacccatctctcaaaa |
| | gtcgagtctctatcactcgagacacatc |
| | caagaaccagttcttcctgcagttgaat |
| | tctgtgactactgaggacacagccacat |
| | attactgtgcaagaggctactatggttt |
| | cggggcctggtttgcttactggggccaa |
| | gggactctggtccctgtctctgca |
| | (SEQ ID NO: 132) |
| huCD37-56 | aagcttgccaccatggggtggagctgca |
| | ttatcctgttcctcgtcgccaccgcaac |
| | cggcgtccactcccaggtgcagctgcaa |
| | gaaagcgggccaggattggtaaaacctt |
| | cccagtctctgagtcttacttgtaccgt |
| | atctggatacagtatcacatctggcttc |
| | gcctggcattggattcgccagtttcccg |
| | gcaagggcttgagtggatggggtatatt |
| | cattattctggaggtaccaactacaacc |
| | cttccctgaagagtcgagtctcaattac |
| | cagggacacttccaagaaccaattcttt |
| | ttgcagcttaattcagtgaccgctgccg |
| | acaccgctacttactactgcgcccgggg |
| | ctactatgggtttggtgcctggttcgcc |
| | tactggggccaggggaccctggtgcccg |
| | tgtcgctgcctccacaaagggccc |
| | (SEQ ID NO: 133) |
| muCD37-57 | gatgtgcagcttcaggagtcaggacctg |
| | acctgttgaaaccttctcagtcactttc |
| | actcacctgcactgtcactggctactcc |
| | atcaccagtggttttgcctggcactgga |
| | tccggcagtttccaggaaacaaactgga |
| | atggatgggctacatactctacagtggt |
| | agcactgtctacagcccatctctcaaaa |
| | gtcgaatctctatcactcgagacacatc |
| | caagaaccagttcttcctgcagttgaat |
| | tctgtgactactgaggacacagccacat |
| | attactgtgcaagagggtactatggtta |
| | cggcgcctggtttgcttactggggccaa |
| | gggactctggtcactgctctgca |
| | (SEQ ID NO: 134) |
| huCD37-57 | aagcttgccaccatgggctggagctgca |
| | tcattctgtttctggtggccacagcaac |
| | tggcgttcacagtcaagtccaactgcag |
| | gagagcggccccggactcctgaaaccat |
| | ctcagtcactcagtctgacatgtactgt |
| | gagcggctacagcattacctcaggcttc |
| | gcttggcattggatcaggcagttcccgg |
| | aaaaggtctggagtggatggggtacatt |
| | ctgtacagcggcagtacagtgtatcacc |
| | ctccttgaaatctaggatatcaatcaca |
| | cgtgatacaagcaaaaatcagttcttcc |
| | tccagctgaactccgtcaccgccgcaga |
| | cacagcaacctattattgtgctcgcgga |
| | tactacggatatggcgcatggttcgcct |
| | attggggccaggggacactcgtgaccgt |
| | ttccgccgcctccacaaagggccc |
| | (SEQ ID NO: 135) |

### Variable Light Chain Polynucleotide Sequences

| | VL Polynucleotide |
|---|---|
| Antibody | Sequence (SEQ ID NO) |
| muCD37-3 | gacatccagatgactcagtctccagcct |
| | ccctttctgtatctgtgggagaaactgt |
| | caccatcacatgtcgagcaagtgagaat |
| | attcgcagtaatttagcatggtatcagc |
| | agaaacagggaaaatctcctcagctcct |
| | ggtcaatgttgcaacaaacttagcagat |
| | ggtgtgccatcaaggttcagtggcagtg |
| | gatcaggcacacagtattccctcaagat |
| | caacagcctgcagtctgaagattttggg |
| | acttattactgtcaacattattggggta |
| | ctacgtggacgttcggtggaggcaccaa |
| | gctggaaatcaaacgt |
| | (SEQ ID NO: 136) |
| chCD37-3 | gaattcgccaccatgagtgtgcccactc |
| | aggtcctggggttgctgctgctgtggct |
| | tacagatgccagatgtgacatccagatg |
| | actcagtctccagcctccctttctgtat |
| | ctgtgggagaaactgtcaccatcacatg |
| | tcgagcaagtgagaatattcgcagtaat |
| | ttagcatggtatcagcagaaacagggaa |
| | aatctcctcagctcctggtcaatgttgc |
| | aacaaacttagcagatggtgtgccatca |
| | aggttcagtggcagtggatcaggcacac |
| | agtattccctcaagatcaacagcctgca |
| | gtctgaagattttgggacttattactgt |
| | caacattattggggtactacgtggacgt |
| | tcggtggaggcaccaagctggaaatcaa |
| | acgtacg (SEQ ID NO: 137) |
| huCD37-3 | gaattcgccaccatgggttggtcctgca |
| (1.0 and 1.1) | tcatcatcttgtttctcgtggccacagc |
| | caccggtgttcactctgatatacaaatg |
| | actcaaagcccttccagtttgagcgtaa |
| | gtgtgggtgaacgcgtaacaatcacctg |
| | tagagctagtgaaaacatccgcagtaat |
| | ctcgcatggtaccaacaaaagccaggta |
| | agtcacctaagctcctcgtgaatgttgc |
| | taccaacctcgctgatggtgtgccttca |
| | cgattctcggttcaggttccggtaccga |
| | ttattcacttaagatcaactcactccaa |
| | ccagaagatttcggtacatattactgtc |
| | aacactactggggtacgacctggacatt |
| | cggtcaaggtactaagctggaaatcaag |
| | cgtacg (SEQ ID NO: 138) |
| muCD37-12 | gacattgtgctaacacagtctcctgctt |
| | ccttagctgtatctctggggcagagggc |
| | caccatctcatgcagggccagccaaagt |
| | gtcagtacatctagctatagttatttgt |
| | actggttccagcagaaaccaggacagcc |
| | acccaaactcctcatcaagtatgcatcc |
| | aacctagcatctggggtccctgccaggt |
| | tcagtggcagtgggtctgggacagactt |
| | caccctcaacatccatcctgtggaggag |
| | gaggatactgcaacatattactgtcaac |
| | acagttgggagattccgtacacgttcgg |
| | aggggggaccaaactggaaataaaacgg |
| | (SEQ ID NO: 139) |
| chCD37-12 | gaattcgccaccatgggttggtcctgta |
| | taatcctgttcttggtggccaccgctac |
| | tggcgttcatagtgatattgtactcact |
| | cagtcaccagccagtctggcagtgtccc |
| | tgggccagcgtgccaccatctcctgccg |
| | ggcctcacagtccgtgagcactagctct |
| | tattcctatctactggtttcaacagaag |
| | ccaggacagccccctaagctgctgatca |
| | agtacgcctccaacctcgccagcggcgt |
| | tcccgctagattctctggttccggtagc |
| | ggaactgatttcactttgaacatccacc |
| | ccgttgaggaagaggataccgccactta |
| | ctattgtcaacactcttgggagattcct |
| | tacacctttggaggaggaacaaagctcg |
| | aaattaagcgtacg |
| | (SEQ ID NO: 140) |
| muCD37-38 | caaattgttctcacccagtccagcaatc |
| | atgtctgcatctccaggggagaaggtca |
| | ccatgacctgcagtgccagctcaagtgt |
| | aacttacatgcactggtaccagcagaag |
| | tcaggcacctcccccaaaagatggattt |
| | atgacacatccaaactggcttctggagt |
| | ccctgctcgcttcagtggcggtgggtct |
| | gggacctcttactctctcacaatcagca |
| | gcatggaggctgaagatgctgccactta |
| | ttactgccagcagtggattagtaaccca |
| | cccacgttcggaggggggaccaagctgg |
| | aaattaaacgg (SEQ ID NO: 141) |
| chCD37-38 | gaattcgccaccatgggctggtcctgta |
| | tcatcctgtttctcgtggccacagctac |
| | aggtgttcattctcagattgtgctgacc |
| | caatcaccagctattatgtccgctagcc |
| | ccggcgagaaagtgacaatgacatgtag |
| | cgctagctcttctgtgacttacatgcat |
| | tggtatcaacagaagtcaggtaccagtc |
| | ccaagcgttggatctacgacacatccaa |
| | actggcctccggagtccctgccaggttc |
| | agcggaggtgggtccggcaccagttatt |
| | cactgaccatatcctctatggaagctga |
| | agatgctgctacttattattgtcaacaa |
| | tggatttctaacccccccacctttggtg |
| | gcggaacaaagctggagatcaagcgtac |
| | g (SEQ ID NO: 142) |
| huCD37-38 | gaattcgccaccatgggatggtcctgca |
| | ttattctgttcttggtcgccactgctac |
| | tggcgttcactctgacattgtgctcaca |
| | cagtctccagcctcaatgtctgcttccc |
| | ccggtgagcgggtgaccatgacatgctc |
| | tgccagttcctccgtgacatatatgcat |
| | tggtatcagcaaaaacccggtacctctc |
| | caaaaagatggatctacgacacttcaaa |
| | gcttgcatcaggcgttcctgccagattt |
| | tccgggtctgggtctggcacttcataca |
| | gtctgaccattagttccatggaagctga |
| | agatgcagccacctattactgcagcagt |
| | ggatttcaaatcctcctaccttcggcgg |
| | cggaacaaactggagataaagcgtacg |
| | (SEQ ID NO: 143) |
| muCD37-50 | caaattgttctcacccagtctccagcaa |
| | tcatgtctgcatctccagggagaaggtc |
| | accatgacctgcagtgccacctcaagtg |
| | tgacttacatgcactggtaccagcagaa |
| | gtcaggcacctcccccaaaagatggatt |
| | tatgacacatccaaactgccttatggag |
| | tccctggtcgtttcagtggtagtgggtc |
| | tgggacctcttactctctcacaatcagc |
| | agcatggaggctgaagatgctgccactt |
| | attactgccagcagtggagtgataaccc |
| | acccacgttcggctcggggacaaagttg |
| | gaaataaagcgg |
| | (SEQ ID NO: 144) |
| huCD37-50 | gaattcgccaccatgggttggtcatgca |
| | ttattctgttcctggttgctaccgcaac |
| | aggagtacatagtgagatagtcctcacc |
| | caaagtcctgctactatgtctgccagcc |
| | caggagagcgtgtgaccatgacttgctc |
| | tgcaacctcaagtgtgacatacatgcat |
| | tggtatcagcaaaagcctggccaatccc |
| | ctaaaaggtggatctacgatacttctaa |
| | tctgccatacggtgtgcccgcaaggttc |
| | tccgggagtggcagtggcaccagttata |
| | gtctgaccatcagttcaatggaagcaga |
| | ggatgcagcaacctattattgtcagcag |
| | tggtccgataatccccctacttttggtc |
| | agggtacaaagctggagattaagcgtac |
| | g (SEQ ID NO: 145) |
| muCD37-51 | caaattgttctcacccagtctccagcaa |
| | tcatgtctgcatctccaggggagaaggt |
| | caccatgacctgcagtgccacctcaagt |
| | gtgacttacatgcactggtaccagcaga |
| | agtcaggcacctcccccaaaagatggat |
| | ttatgacacatccaaactggcttctgga |
| | gtccctgctcgcttcagtggctagtggg |
| | tctgggacctcttactctctcacaatca |
| | gcaacatggaggctgaagatgctgccac |
| | ttattactgccagcagtggagtagtaac |
| | ccacccacgttcggctcggggcaaagtt |
| | ggaaataaagcgg |
| | (SEQ ID NO: 146) |
| huCD37-51 | gaattcgccaccatgggatggagctgta |
| | ttattctgttcctggttgctactgctac |
| | tggcgtccattccgagatagtcctcacc |
| | cagagccccgcaaccatgagtgcctccc |
| | ctggggagcgagtgactatgacttgttc |
| | cgccacttcttcagttacctatatgcat |
| | tggtatcagcagaaacctggacagtctc |
| | caaagcgttggatttacgacacctccaa |
| | cctggcttcaggagttcctgctaggttc |
| | agcggatctgggtctggcacaagttatt |
| | cactcaccattagttccatggaggccga |
| | agatgccgctacttactactgtcagcag |
| | tggagcagcaacccccctacattcgggc |
| | agggaactaagctggagatcaaacgtac |
| | g (SEQ ID NO: 147) |
| muCD37-56 | caaattgttctcacccagtctccagcat |
| | tcatgtctgcatctccaggggataaggt |
| | caccatgacctgcagtgccagttcaagt |
| | gttacttacatgcactggtatcagcaga |
| | agtcaggcacctcccccaaaagatggat |
| | ttatgacacatccaaactggcttctgga |
| | gtccctgctcgcttcagtggcggtgggt |
| | ctgggacctcttactctctcacaatcag |
| | caccatggaggctgaagatgctgccact |
| | tattactgccagcagtggattagtgacc |
| | cacccacgttcggaggggggaccaagct |
| | ggaaataaaacgg |
| | (SEQ ID NO: 148) |
| huCD37-56 | gaattcgccaccatgggctggtcctgta |
| | tcatcctgtttctggtggcaaccgctac |
| | tggggttcactctgatattgtcctgaca |
| | cagagtccagccttcatgagtgcttctc |
| | ccggagaaaaggtcacaatgacttgttc |
| | agcttcctcctccgtcacatacatgcat |
| | tggtaccagcagaagcctgaccagagtc |
| | ctaagaggtggatctatgatacaagcaa |
| | tctggcttccggtgtccctcccgctttt |
| | caggcggcggaagcggaactgactatag |
| | ccttaccatctcctcaatggaagccgag |
| | gacgctgctacatattactgccagcaat |
| | ggatcagcgaccctcctactttcggaca |
| | gggaacaaaattggaaattaagcgtacg |
| | (SEQ ID NO: 149) |
| muCD37-57 | caaattgttctcacccagtctccagcaa |
| | tcatgtctgcatctccaggggagaaggt |
| | caccatgacctgcagtgccacctcaagt |
| | gtgacttacatgcactggtaccagcaga |
| | agtcaggcacctcccccaaaagatggat |
| | ttatgacacatccaaactggcttctgga |
| | gtccctgctcgcttcagtggcagtgggt |
| | ctgggacctcttactctctcacaatcag |
| | cagcatggaggctgaagatgctgccact |
| | tattactgccagcagtggagtgataacc |
| | cacccacgttcggctcggggacaaagtt |
| | ggaaataaagcgg |
| | (SEQ ID NO: 150) |
| huCD37-57 | gaattcgccaccatggggtggtcctgta |
| | ttatcctgttcctggtcgcaaccgccac |
| | aggcgttcactccgagatcgtgttgact |
| | cagagcccagccaccatgtccgcttccc |
| | ccggggagagagtgacaatgacttgttc |
| | cgccacaagttctgtaacctacatgcat |
| | tggtaccagcaaaaaccaggacagagtc |
| | cccgtcgttggatttatgatacctctaa |
| | cctggcttcaggcgttcctgcccgcttt |
| | tctggtagtggatctgggacttcctata |
| | gccttaccataagctctatggaagccga |
| | ggacgccgctacatactactgccagcag |
| | tggagtgataacccccccaccttcgggc |
| | agggaaccaaattggagatcaaacgtac |
| | g (SEQ ID NO: 151) |

### Full-Length Heavy Chain Polynucleotide Sequences

| Anti- | |
|---|---|
| body | Full-Length Heavy Chain Polynucleotide Sequence (SEQ ID NO) |
| chCD37- | aagcttgccaccatggctgtcctggcactgctcctctgcctggtgacatacccaagctgtgtcctatcacaggtgcaggtg |
| 3 | aaggagtcaggacctggcctggtggcgccctcacagagcctgtccattacatgcactgtctcagggttctcattaaccac |
| | ctctggtgtaagctgggttcgccagcctccaggaaagggtctggagtggctgggagtaatatggggtgacgggagcac |
| | aaactatcattcagctctcaaatccagactgagcatcaagaaggatcactccaagagccaagttttcttaaaactgaacagt |
| | ctgcaaactgatgacacagccacgtactactgtgccaaaggaggctactcgttggctcactggggccaagggactctgg |
| | tcacagtctctgcagcctctacgaagggcccatcagttttccccttggctccaagttctaaatccacaagcggtggaacag |
| | ctgcactgggatgcctcgttaaagattatttccctgagcctgtgacagtgagctggaatagcggagcattgacttcaggtgt |
| | gcacacttttcccgctgtgttgcagtcctccggtctgtactcactgtccagtgtcgtaaccgtcccttctagcagcttgggaa |
| | cccagacctacatctgtaacgtcaaccataaaccatccaacacaaaggtggataagaaggttgaaccaaagagctgtga |
| | taagacacatacatgccctccttgtcctgcaccagagctcctcggaggtccatctgtgttcctgtttccccccaaacccaag |
| | gacactcttatgatctctcgtactccagaggtcacctgtgttgttgtcgacgtgagccatgaagatcccgaggttaaattcaa |
| | ctggtacgtggatggagtcgaggttcacaatgccaagaccaagcccagggaggagcaatataattctacatatcgggta |
| | gtgagcgttctgaccgtgctccaccaagattggctcaatggaaaagagtacaagtgcaaggtgtccaacaaggctcttcc |
| | cgctcccattgagaaaactatctccaaagccaaggggcagccacgggaaccccaggtgtatacattgcccccatctaga |
| | gacgagctgaccaagaaccaggtgagtctcacttgtctggtcaaggggttttacccttctgacattgctgtagagtgggag |
| | tctaacggacagccagaaaacaactacaagacaactcccccagtgctggacagcgacgggagcttcttcctctactcca |
| | agttgactgtagacaagtctagatggcagcaaggaaacgttttctcctgctcagtaatgcatgaggctctgcacaatcacta |
| | tacccagaaatcactgtcccttagcccagggtgactcgag (SEQ ID NO: 152) |
| huCD37- | aagcttgccaccatgggttggagctgcattattctgtttctggtggccaccgccaccggtgtgcactcacaagtccaagtc |
| 3v1.0 | caagaatctggtccaggtctggtggccccttcccaaactctgagcatcacctgtaccgtttctggttttagccttaccacctc |
| | tggtgtgagttgggtacgccaaccacccggtaagggtctcgaatggctgggtgtaatctggggtgatggttccacaaatt |
| | accatccttccctcaagtcccgccttagcatcaaaaaggatcacagcaaaagtcaagttttcctgaaactgaatagtctgac |
| | agcagccgatacagccacctactattgcgccaagggtggttatagtcttgcacactggggtcaaggtaccctcgttaccgt |
| | ctcctcagctagtaccaagggcccatcagttttccccttggctccaagttctaaatccacaagcggtggaacagctgcact |
| | gggatgcctcgttaaagattatttccctgagcctgtgacagtgagctggaatagcggagcattgacttcaggtgtgcacac |
| | ttttcccgctgtgttgcagtcctccggtctgtactcactgtccagtgtcgtaaccgtcccttctagcagcttgggaacccaga |
| | cctacatctgtaacgtcaaccataaaccatccaacacaaaggtggataagaaggttgaaccaaagagctgtgataagac |
| | acatacatgccctccttgtcctgcaccagagctcctcggaggtccatctgtgttcctgtttccccccaaacccaaggacact |
| | cttatgatctctcgtactccagaggtcacctgtgttgttgtcgacgtgagccatgaagatcccgaggttaaattcaactggta |
| | cgtggatggagtcgaggttcacaatgccaagaccaagcccagggaggagcaatataattctacatatcgggtagtgagc |
| | gttctgaccgtgctccaccaagattggctcaatggaaaagagtacaagtgcaaggtgtccaacaaggctcttcccgctcc |
| | cattgagaaaactatctccaaagccaaggggcagccacgggaaccccaggtgtatacattgcccccatctagagacga |
| | gctgaccaagaaccaggtgagtctcacttgtctggtcaaggggttttacccttctgacattgctgtagagtgggagtctaac |
| | ggacagccagaaaacaactacaagacaactcccccagtgctggacagcgacgggagcttcttcctctactccaagttga |
| | ctgtagacaagtctagatggcagcaaggaaacgttttctcctgctcagtaatgcatgaggctctgcacaatcactataccc |
| | agaaatcactgtcccttagcccagggtgactcgag (SEQ ID NO: 153) |
| huCD37- | aagcttgccaccatgggctggagctgtatcattctgtttctggtggcgacagctactggggtccactcccaagtgcaggta |
| 3v1.1 | caagagtccgggcctggattggtcgcaccaagccagaccctctctatcacttgtaccgttagcgggttctctctgacaacc |
| | agtggagtgagttgggtgaggcagccaccaggaaagggactggagtggctgggggtgatttggggcgacggcagca |
| | caaactatcattccagtcttaaatctcggttgtccattaaaaaagaccatagtaaatctcaagttttcctgaaactcaatag |
| | cctgacagccgcagacactgctacgtattactgcgccaaaggaggatacagtctggctcactggggacaggggaccctggt |
| | gaccgtgtcatccgcatcaacaaagggcccatcagttttccccttggctccaagttctaaatccacaagcggtggaacag |
| | ctgcactgggatgcctcgttaaagattatttccctgagcctgtgacagtgagctggaatagcggagcattgacttcaggtgt |
| | gcacacttttcccgctgtgttgcagtcctccggtctgtactcactgtccagtgtcgtaaccgtcccttctagcagcttgggaa |
| | cccagacctacatctgtaacgtcaaccataaaccatccaacacaaaggtggataagaaggttgaaccaaagagctgtga |
| | taagacacatacatgccctccttgtcctgcaccagagctcctcggaggtccatctgtgttcctgtttccccccaaacccaag |
| | gacactcttatgatctctcgtactccagaggtcacctgtgttgttgtcgacgtgagccatgaagatccgaggttaaattcaa |
| | ctggtacgtggatggagtcgaggttcacaatgccaagaccaagcccagggaggagcaatataattctacatatcgggta |
| | gtgagcgttctgaccgtgctccaccaagattggctcaatggaaaagagtacaagtgcaaggtgtccaacaaggctcttcc |
| | cgctcccattgagaaaactatctccaaagccaaggggcagccacgggaaccccaggtgtatacattgcccccatctaga |
| | gacgagctgaccaagaaccaggtgagtctcacttgtctggtcaaggggttttacccttctgacattgctgtagagtggag |
| | tctaacggacagccagaaaacaactacaagacaactcccccagtgctggacagcgacgggagcttcttcctctactcca |
| | agttgactgtagacaagtctagatggcagcaaggaaacgttttctcctgctcagtaatgcatgaggctctgcacaatcacta |
| | tacccagaaatcactgtcccttagcccagggtgactcgag (SEQ ID NO: 154) |
| chCD37- | aagcttgccaccatggggtggtcatgcataatcctctttctggtcgctactgctaccggtgtgcactcacagattcagctgg |
| 12 | ttcaaagtggcccagagctgaaaaagccaggggaaacagtgaaaataagttgcaaggcatccggttacactttcacaaa |
| | gtacggcatgaactgggtcaagcaggcccagggcaaggggctcaaatggatgggttggatcaataccaacactggcg |
| | agtctaggaatgctgaggagtttaagggccggtttgccttcagcctggagacaagtgccagcacagcttacctgcaaatc |
| | aacaatctgaagtatgaggatacagcaacctatttctgcggccgcggcactgtcgttgcagactggggacaaggtacca |
| | ccttgactgtatccagtgccagcactaagggcccatcagttttccccttggctccaagttctaaatccacaagcggtggaa |
| | cagctgcactgggatgcctcgttaaagattatttccctgagcctgtgacagtgagctggaatagcggagcattgacttcag |
| | gtgtgcacacttttcccgctgtgttgcagtcctccggtctgtactcactgtccagtgtcgtaaccgtcccttctagcagct |
| | tgggaacccagacctacatctgtaacgtcaaccataaaccatccaacacaaaggtggataagaaggttgaaccaaagagctg |
| | tgataagacacatacatgccctccttgtcctgcaccagagctcctcggaggtccatctgtgttcctgtttccccccaaaccc |
| | aaggacactcttatgatctctcgtactccagaggtcacctgtgttgttgtcgacgtgagccatgaagatcccgaggttaaatt |
| | caactggtacgtggatggagtcgaggttcacaatgccaagaccaagcccagggaggagcaatataattctacatatcgg |
| | gtagtgagcgttctgaccgtgctccaccaagattggctcaatggaaaagagtacaagtgcaaggtgtccaacaaggctct |
| | tcccgctcccattgagaaaactatctccaaagccaaggggcagccacgggaaccccaggtgtatacattgcccccatct |
| | agagacgagctgaccaagaaccaggtgagtctcacttgtctggtcaaggggttttacccttctgacattgctgtagagtgg |
| | gagtctaacggacagccagaaaacaactacaagacaactcccccagtgctggacagcgacgggagcttcttcctctact |
| | ccaagttgactgtagacaagtctagatggcagcaaggaaacgttttctcctgctcagtaatgcatgaggctctgcacaatc |
| | actatacccagaaatcactgtcccttagggtgactcgag (SEQ ID NO: 155) |
| chCD37- | aagcttgccaccatgggctggagttgtatcattctgtttttggtggccaccgccactggagtccattcccaagtgcaactcc |
| 38 | aggaatctggccctgacctggttaagccatctcagagcctctccctgacctgcactgttacaggatactcaatcacatcag |
| | gctttggctggcactggatcagacaatttcccgggaacaagttggaatggatggcttacattctgtatagcgggggtaccg |
| | attacaatccttccctcaagagccgaatctctatcaccagggatacaagcaagaaccaattttttctccgcctcagctctgtg |
| | actaccgaagataccgctacttactattgtgccaggggctactatggatatggtgcatggttcgtctattggggccaggga |
| | accctggtgactgtgagcgctgcctctaccaagggcccatcagttttccccttggctccaagttctaaatccacaagcggt |
| | ggaacagctgcactgggatgcctcgttaaagattatttccctgagcctgtgacagtgagctggaatagcggagcattgact |
| | tcaggtgtgcacacttttcccgctgtgttgcagtcctccggtctgtactcactgtccagtgtcgtaaccgtcccttctagcag |
| | cttgggaacccagacctacatctgtaacgtcaaccataaaccatccaacacaaaggtggataagaaggttgaaccaaag |
| | agctgtgataagacacatacatgccctccttgtcctgcaccagagctcctcggaggtccatctgtgttcctgtttcccccca |
| | aacccaaggacactcttatgatctctcgtactccagaggtcacctgtgttgttgtcgacgtgagccatgaagatcccgagg |
| | ttaaattcaactggtacgtggatggagtcgaggttcacaatgccaagaccaagcccagggaggagcaatataattctaca |
| | tatcgggtagtgagcgttctgaccgtgctccaccaagattggctcaatggaaaagagtacaagtgcaaggtgtccaacaa |
| | ggctcttcccgctcccattgagaaaactatctccaaagccaaggggcagccacgggaaccccaggtgtatacattgccc |
| | ccatctagagacgagctgaccaagaaccaggtgagtctcacttgtctggtcaaggggttttacccttctgacattgctgtag |
| | agtgggagtctaacggacagccagaaaacaactacaagacaactcccccagtgctggacagcgacgggagcttcttcc |
| | tctactccaagttgactgtagacaagtctagatggcagcaaggaaacgttttctcctgctcagtaatgcatgaggctctgca |
| | caatcactatacccagaaatcactgtcccttagcccagggtgactcgag (SEQ ID NO: 156) |
| huCD37- | aagcttgccaccatgggttggagctgcatcattcttttcctggtcgctactgcaactggagtccactcacaggtccagctgc |
| 38 | aagagtccggtcctgggcttgtgaaacccagccagtccctcagtctcacctgtactgtctctggctactctattaccagtgg |
| | gttcggctggcattggattaggcagtttcccggtaaggggctggagtggatggcatatatcctgtacagcggaggaacc |
| | gattacaacccaagtctgaagagcaggatcagcattacccgggacacaagcaaaaaccagtttttccttcggctgtctagt |
| | gttacagctgcagacaccgctacttactattgtgctcggggttactatggctatggggcttggtttgtgtattggggacaag |
| | gcactcttgtgaccgtgagcagcgcctcaacaaagggcccatcagttttccccttggctccaagttctaaatccacaagcg |
| | gtggaacagctgcactgggatgcctcgttaaagattatttccctgagcctgtgacagtgagctggaatagcggagcattg |
| | acttcaggtgtgcacacttttcccgctgtgttgcagtcctccggtctgtactcactgtccagtgtcgtaaccgtcccttct |
| | agcagcttgggaacccagacctacatctgtaacgtcaaccataaaccatccaacacaaaggtggataagaaggttgaaccaa |
| | agagctgtgataagacacatacatgccctccttgtcctgcaccagagctcctcggaggtccatctgtgttcctgtttccccc |
| | caaacccaaggacactcttatgatctctcgtactccagaggtcacctgtgttgttgtcgacgtgagccatgaagatcccga |
| | ggttaaattcaactggtacgtggatggagtcgaggttcacaatgccaagaccaagcccagggaggagcaatataattcta |
| | catatcgggtagtgagcgttctgaccgtgctccaccaagattggctcaatggaaaagagtacaagtgcaaggtgtccaac |
| | aaggctcttcccgctcccattgagaaaactatctccaaagccaaggggcagccacgggaaccccaggtgtatacattgc |
| | ccccatctagagacgagctgaccaagaaccaggtgagtctcacttgtctggtcaaggggttttacccttctgacattgctgt |
| | agagtgggagtctaacggacagccagaaaacaactacaagacaactcccccagtgctggacagcgacgggagcttctt |
| | cctctactccaagttgactgtagacaagtctagatggcagcaaggaaacgttttctcctgctcagtaatgcatgaggctctg |
| | cacaatcactatacccagaaatcactgtcccttagcccagggtgactcgag (SEQ ID NO: 157) |
| huCD37- | aagcttgccaccatggggtggtcctgcataatccttttcctggttgctactgctaccggagtccattcacaggtgcagctgc |
| 50 | aggagtccggccccggcctgctcaagccttctcagagtctgagtctgacttgtactgtttctggctacagcataaccagcg |
| | gtttcgcttggcactggatcagacagcatcccggcaacaaactggagtggatgggatacatactgtactcaggctcaact |
| | gtctattccccctccctgaaatcccggatcagtattacccgtgacacttctaagaaccatttttttctgcagctgaacagcg |
| | ttaccgcagctgacactgcaacctactactgtgcccggggatattatggatacggagcttggttcgcttactggggccaagg |
| | caccctcgtaactgtgagtgctgcttccaccaagggcccatcagttttccccttggctccaagttctaaatccacaagcggt |
| | ggaacagctgcactgggatgcctcgttaaagattatttccctgagcctgtgacagtgagctggaatagcggagcattgact |
| | tcaggtgtgcacacttttcccgctgtgttgcagtcctccggtctgtactcactgtccagtgtcgtaaccgtcccttctagcag |
| | cttgggaacccagacctacatctgtaacgtcaaccataaaccatccaacacaaaggtggataagaaggttgaaccaaag |
| | agctgtgataagacacatacatgccctccttgtcctgcaccagagctcctcggaggtccatctgtgttcctgtttcccccca |
| | aacccaaggacactcttatgatctctcgtactccagaggtcacctgtgttgttgtcgacgtgagccatgaagatcccgagg |
| | ttaaattcaactggtacgtggatggagtcgaggttcacaatgccaagaccaagcccagggaggagcaatataattctaca |
| | tatcgggtagtgagcgttctgaccgtgctccaccaagattggctcaatggaaaagagtacaagtgcaaggtgtccaacaa |
| | ggctcttcccgctcccattgagaaaactatctccaaagccaaggggcagccacgggaaccccaggtgtatacattgccc |
| | ccatctagagacgagctgaccaagaaccaggtgagtctcacttgtctggtcaaggggttttacccttctgacattgctgtag |
| | agtgggagtctaacggacagccagaaaacaactacaagacaactcccccagtgctggacagcgacgggagcttcttcc |
| | tctactccaagttgactgtagacaagtctagatggcagcaaggaaacgttttctcctgctcagtaatgcatgaggctctgca |
| | caatcactatacccagaaatcactgtcccttagcccaggtgactcgag (SEQ ID NO: 158) |
| huCD37- | aagcttgccaccatgggttggtcttgcatcatcctgttcctggtggccactgccactggcgtgcattcagaagttcagttggt |
| 51 | ggagtccggcccagaagtgctgaaacccggcgaatcactgtccctgacttgtaccgtgtcaggttatagcatcagcagc |
| | ggctttgcttggcactggattcggcagtttccaggcaagggactggaatggatgggctacatccattacagtggctcaac |
| | caattacagccctagcctgcagggccgaatctctattaccagggatagttctattaaccagtttttcctgcagcttaattcc |
| | gtgactgcctctgacacagcaacttactattgcgcccgtggctactacgggttcggagcctggtttgtatactggggtcaggg |
| | caccctggtcactgtctcagccgcctctaccaagggcccatcagttttccccttggctccaagttctaaatccacaagcgg |
| | ggaacagctgcactgggatgcctcgttaaagattatttccctgagcctgtgacagtgagctggaatagcggagcattgact |
| | tcaggtgtgcacacttttcccgctgtgttgcagtcctccggtctgtactcactgtccagtgtcgtaaccgtcccttctagcag |
| | cttgggaacccagacctacatctgtaacgtcaaccataaaccatccaacacaaaggtggataagaaggttgaaccaaag |
| | agctgtgataagacacatacatgccctccttgtcctgcaccagagctcctcggaggtccatctgtgttcctgtttcccccca |
| | aacccaaggacactcttatgatctctcgtactccagaggtcacctgtgttgttgtcgacgtgagccatgaagatcccgagg |
| | ttaaattcaactggtacgtggatggagtcgaggttcacaatgccaagaccaagcccagggaggagcaatataattctaca |
| | tatcgggtagtgagcgttctgaccgtgctccaccaagattggctcaatggaaaagagtacaagtgcaaggtgtccaacaa |
| | ggctcttcccgctcccattgagaaaactatctccaaagccaaggggcagccacgggaaccccaggtgtatacattgccc |
| | ccatctagagacgagctgaccaagaaccaggtgagtctcacttgtctggtcaaggggttttacccttctgacattgctgtag |
| | agtgggagtctaacggacagccagaaaacaactacaagacaactcccccagtgctggacagcgacgggagcttcttcc |
| | tctactccaagttgactgtagacaagtctagatggcagcaaggaaacgttttctcctgctcagtaatgcatgaggctctgca |
| | caatcactatacccagaaatcactgtcccttagcccagggtgactcgagg (SEQ ID NO: 159) |
| huCD37- | aagcttgccaccatggggtggagctgcattatcctgttcctcgtcgccaccgcaaccggcgtccactcccaggtgcagct |
| 56 | gcaagaaagcgggccaggattggtaaaaccttcccagtctctgagtcttacttgtaccgtatctggatacagtatcacatct |
| | ggcttcgcctggcattggattcgccagtttcccggcaaggggcttgagtggatggggtatattcattattctggaggtacca |
| | actacaacccttccctgaagagtcgagtctcaattaccagggacacttccaagaaccaattctttttgcagcttaattcagtg |
| | accgctgccgacaccgctacttactactgcgcccggggctactatgggtttggtgcctggttcgcctactggggccaggg |
| | gaccctggtgcccgtgtctgctgcctccacaaagggcccatcagttttccccttggctccaagttctaaatccacaagcgg |
| | tggaacagctgcactgggatgcctcgttaaagattatttccctgagcctgtgacagtgagctggaatagcggagcattgac |
| | ttcaggtgtgcacacttttcccgctgtgttgcagtcctccggtctgtactcactgtccagtgtcgtaaccgtcccttctagc |
| | agcttgggaacccagacctacatctgtaacgtcaaccataaaccatccaacacaaaggtggataagaaggttgaaccaaag |
| | agctgtgataagacacatacatgccctccttgtcctgcaccagagctcctcggaggtccatctgtgttcctgtttcccccca |
| | aacccaaggacactcttatgatctctcgtactccagaggtcacctgtgttgttgtcgacgtgagccatgaagatcccgagg |
| | ttaaattcaactggtacgtggatggagtcgaggttcacaatgccaagaccaagcccagggaggagcaatataattctaca |
| | tatcgggtagtgagcgttctgaccgtgctccaccaagattggctcaatggaaaagagtacaagtgcaaggtgtccaacaa |
| | ggctcttcccgctcccattgagaaaactatctccaaagccaaggggcagccacgggaaccccaggtgtatacattgccc |
| | ccatctagagacgagctgaccaagaaccaggtgagtctcacttgtctggtcaaggggttttacccttctgacattgctgtag |
| | agtgggagtctaacggacagccagaaaacaactacaagacaactcccccagtgctggacagcgacgggagcttcttcc |
| | tctactccaagttgactgtagacaagtctagatggcagcaaggaaacgttttctcctgctcagtaatgcatgaggctctgca |
| | caatcactatacccagaaatcactgtcccagggtgactcgag (SEQ ID NO: 160) |
| huCD37- | aagcttgccaccatgggctggagctgcatcattctgtttctggtggccacagcaactggcgttcacagtcaagtccaactg |
| 57 | caggagagcggccccggactcctgaaaccatctcagtcactcagtctgacatgtactgtgagcggctacagcattacctc |
| | aggcttcgcttggcattggatcaggcagttccccggaaaaggtctggagtggatggggtacattctgtacagcggcagta |
| | cagtgtattcaccctccttgaaatctaggatatcaatcacacgtgatacaagcaaaaatcagttcttcctccagctgaactcc |
| | gtcaccgccgcagacacagcaacctattattgtgctcgcggatactacggatatggcgcatggttcgcctattggggcca |
| | ggggacactcgtgaccgtttccgccgcctccacaaagggcccatcagttttccccttggctccaagttctaaatccacaag |
| | cggtggaacagctgcactgggatgcctcgttaaagattatttccctgagcctgtgacagtgagctggaatagcggagcat |
| | tgacttcaggtgtgcacactttcccgctgtgttgcagtcctccggtctgtactcactgtccagtgtcgtaaccgtccctcta |
| | gcagcttgggaacccagacctacatctgtaacgtcaaccataaaccatccaacacaaaggtggataagaaggttgaacc |
| | aaagagctgtgataagacacatacatgccctccttgtcctgcaccagagctcctcggaggtccatctgtgttcctgtttccc |
| | cccaaacccaaggacactcttatgatctacgtactccagaggtcacctgtgrtgttgtcgacgtgagccatgaagatccc |
| | gaggttaaattcaactggtacgtggatggagtcgaggttcacaatgccaagaccaagcccagggaggagcaatataatt |
| | ctacatatcgggtagtgagcgttctgaccgtgctccaccaagattggctcaatggaaaagagtacaagtgcaaggtgtcc |
| | aacaaggctcttcccgctcccattgagaaaactatctccaaagccaaggggcagccacgggaaccccaggtgtatacat |
| | tgcccccatctagagacgagctgaccaagaaccaggtgagtctcacttgtctggtcaaggggttttacccttctgacattg |
| | ctgagagtgggagtctaacggacagccagaaaacaactacaagacaactcccccaggctggacagcgacgggagc |
| | ttcttcctctactccaagttgactgtagacaagtctagatggcagcaaggaaacgtatctcctgctcagtaatgcatgaggc |
| | tctgcacaatcactatacccagaaatcactgtcccttagcccagggtgactgag (SEQ ID NO: 161) |

### Full-Length Light Chain Polynucleotide Sequences

| Anti- | |
|---|---|
| body | Full-lenth Light Chain Polynucleotide Sequence (SEQ ID NO) |
| chCD37- | gaattcgccaccatgagtgtgcccactcaggtcctggggttgctgctgctgtggcttacagatgccagatgtgacatccag |
| 3 | atgactcagtctccagcctccctttctgtatctgtgggagaaactgtcaccatcacatgtcgagcaagtgagaatattcgca |
| | gtaatttagcatggtatcagcagaaacagggaaaatctcctcagctcctggtcaatgttgcaacaaacttagcagatggtgt |
| | gccatcaaggttcagtggcagtggatcaggcacacagtattccctcaagatcaacagcctgcagtctgaagattttggga |
| | cttattactgtcaacattattggggtactacgtggacgttcggtggaggcaccaagctggaaatcaaacgtacggtggctg |
| | caccatctgtcttcatcttcccgccatctgatgagcagttgaaatctggaactgcctctgttgtgtgcctgctgaataactt |
| | ctatcccagagaggccaaagtacagtggaaggtggataacgccctccaatcgggtaactcccaggagagtgtcacagagc |
| | aggacagcaaggacagcacctacagcctcagcagcaccctgacgctgagcaaagcagactacgagaaacacaaagt |
| | ctacgcctgcgaagtcacccatcagggcctgagctcgcccgtcacaaagagcttcaacaggggagagtgttag |
| | (SEQ ID NO: 162) |
| huCD37- | gaattcgccaccatgggttggtcctgcatcatcttgtttctcgtggccacagccaccggtgttcactctgatatacaaatgac |
| 3 | tcaaagcccttccagtttgagcgtaagtgtgggtgaacgcgtaacaatcacctgtagagctagtgaaaacatccgcagta |
| (1.0 | atctcgcatggtaccaacaaaagccaggtaagtcacctaagctcctcgtgaatgttgctaccaacctcgctgatggtgtgc |
| and | cttcacgattctctggttcaggttccggtaccgattattcacttaagatcaactcactccaaccagaagatttcggtacata |
| 1.1) | ttactgtcaacactactggggtacgacctggacattcggtcaaggtactaagctggaaatcaagcgtacggtggctgcaccat |
| | ctgtcttcatcttcccgccatctgatgagcagttgaaatctggaactgcctctgttgtgtgcctgctgaataacttctatccc |
| | agagaggccaaagtacagtggaaggtggataacgccctccaatcgggtaactcccaggagagtgtcacagagcaggac |
| | agcaaggacagcacctacagcctcagcagcaccctgacgctgagcaaagcagactacgagaaacacaaagtctacgc |
| | ctgcgaagtcacccatcagggcctgagctcgcccgtcacaaagagcttcaacaggggagagtgttag(SEQ ID |
| | NO: 163) |
| chCD37- | gaattcgccaccatgggttggtcctgtataatcctgttcttggtggccaccgctactggcgttcatagtgatattgtactcac |
| 12 | tcagtcaccagccagtctggcagtgtccctgggccagcgtgccaccatctcctgccgggcctcacagtccgtgagcacta |
| | gctcttattcctatctctactggtttcaacagaagccaggacagccccctaagctgctgatcaagtacgcctccaacctcgc |
| | cagcggcgttcccgctagattctctggttccggtagcggaactgatttcactttgaacatccaccccgttgaggaagagga |
| | taccgccacttactattgtcaacactcttgggagattccttacacctttggaggaggaacaaagctcgaaattaagcgtacg |
| | gtggctgcaccatctgtcttcatcttcccgccatctgatgagcagttgaaatctggaactgcctctgttgtgtgcctgctgaa |
| | taacttctatcccagagaggccaaagtacagtggaaggtggataacgccctccaatcgggtaactcccaggagagtgtca |
| | cagagcaggacagcaaggacagcacctacagcctcagcagcaccctgacgctgagcaaagcagactacgagaaaca |
| | caaagtctacgcctgcgaagtcacccatcagggcctgagctcgcccgtcacaaagagcttcaacaggggagagtgttag |
| | (SEQ ID NO: 164) |
| chCD37- | gaattcgccaccatgggctggtcctgtatcatcctgtttctcgtggccacagctacaggtgttcattctcagattgtgctgac |
| 38 | ccaatcaccagctattatgtccgctagccccggcgagaaagtgacaatgacatgtagcgctagctcttctgtgacttacat |
| | gcattggtatcaacagaagtcaggtaccagtcccaagcgttggatctacgacacatccaaactggcctccggagtccctg |
| | ccaggttcagcggaggtgggtccggcaccagttattcactgaccatatcctctatggaagctgaagatgctgctacttatta |
| | ttgtcaacaatggatttctaacccccccacctnggtggcggaacaaagctggagatcaagcgtacggtggctgcaccat |
| | ctgtcttcatcttcccgccatctgatgagcagttgaaatctggaactgcctctgttgtgtgcctgctgaataacttctatccc |
| | agagaggccaaagtacagtggaaggtggataacgccctccaatcgggtaactcccaggagagtgtcacagagcaggac |
| | agcaaggacagcacctacagcctcagcagcaccctgacgctgagcaaagcagactacgagaaacacaaagtctacgc |
| | ctgcgaagtcacccatcagggcctgagctcgcccgtcacaaagagcttcaacaggggagagtgttag(SEQ ID |
| | NO: 165) |
| huCD38- | gaattcgccaccatgggatggtcctgcattattctgttcttggtcgccactgctactggcgttcactctgacattgtgctcac |
| 38 | acagtctccagcctcaatgtctgcttcccccggtgagcgggtgaccatgacatgctctgccagttcctccgtgacatatatgc |
| | attggtatcagcaaaaacccggtacctctccaaaaagatggatctacgacacttcaaagcttgcatcaggcgttcctgcca |
| | gattttccgggtctgggtctggcacttcatacagtctgaccattagttccatggaagctgaagatgcagccacctattactgt |
| | cagcagtggatttcaaatcctcctaccttcggcggcggaaccaaactggagataaagcgtacggtggctgcaccatctgt |
| | cttcatcttcccgccatctgatgagcagttgaaatctggaactgcctctgttgtgtgcctgctgaataacttctatcccagag |
| | aggccaaagtacagtggaaggtggataacgccctccaatcgggtaactcccaggagagtgtcacagagcaggacagca |
| | aggacagcacctacagcctcagcagcaccctgacgctgagcaaagcagactacgagaaacacaaagtctacgcctgc |
| | gaagtcacccatcagggcctgagctcgcccgtcacaaagagcttcaacaggggagagtgttag(SEQ ID |
| | NO: 166) |
| huCD37- | gaattcgccaccatgggttggtcatgcattattctgttcctggttgctaccgcaacaggagtacatagtgagatagtcctcac |
| 50 | ccaaagtcctgctactatgtctgccagcccaggagagcgtgtgaccatgacttgctctgcaacctcaagtgtgacatacat |
| | gcattggtatcagcaaaagcctggccaatcccctaaaaggtggatctacgatacttctaatctgccatacggtgtgcccgc |
| | aaggttctccgggagtggcagtggcaccagttatagtctgaccatcagttcaatggaagcagaggatgcagcaacctatt |
| | attgtcagcagtggtccgataatccccctacttttggtcagggtacaaagctggagattaagcgtacggtggctgcaccat |
| | ctgtcttcatcttcccgccatctgatgagcagttgaaatctggaactgcctctgttgtgtgcctgctgaataacttctatccc |
| | agagaggccaaagtacagtggaaggtggataacgccctccaatcgggtaactcccaggagagtgtcacagagcaggac |
| | agcaaggacagcacctacagcctcagcagcaccctgacgctgagcaaagcagactacgagaaacacaaagtctacgc |
| | ctgcgaagtcacccatcagggcctgagctcgcccgtcacaaagagcttcaacaggggagagtgttag(SEQ ID |
| | NO: 167) |
| huCD37- | gaattcgccaccatgggatggagctgtattattctgttcctggttgctactgctactggcgtccattccgagatagtcctcac |
| 51 | ccagagccccgcaaccatgagtgcctcccctggggagcgagtgactatgacttgttccgccacttcttcagttacctatat |
| | gcattggtatcagcagaaacctggacagtctccaaagcgttggantacgacacctccaacctggcttcaggagttcctgc |
| | taggttcagcggatctgggtctggcacaagttattcactcaccattagttccatggaggccgaagatgccgctacttactac |
| | tgtcagcagtggagcagcaacccccctacattcgggcagggaactaagctggagatcaaacgtacggtggctgcacca |
| | tctgtcttcatcttcccgccatctgatgagcagttgaaatctggaactgcctctgttgtgtgcctgctgaataacttctat |
| | cccagagaggccaaagtacagtggaaggtggataacgccctccaatcgggtaactcccaggagagtgtcacagagcaggac |
| | agcaaggacagcacctacagcctcagcagcaccctgacgctgagcaaagcagactacgagaaacacaaagtctacgc |
| | ctgcgaagtcacccatcagggcctgagctcgcccgtcacaaagagcncaacaggggagagtgttag(SEQ ID |
| | NO: 168) |
| huCD37- | gaattcgccaccatgggctggtcctgtatcatcctgtttctggtggcaaccgctactggggttcactctgatattgtcctgac |
| 56 | acagagtccagccttcatgagtgcnctcccggagaaaaggtcacaatgacttgttcagcttcctcctccgtcacatacatg |
| | cattggtaccagcagaagcctgaccagagtcctaagaggtggatctatgatacaagcaatctggcttccggtgtcccctc |
| | ccgcttttcaggcggcggaagcggaactgactatagccttaccatctcctcaatggaagccgaggacgctgctacatatt |
| | actgccagcaatggatcagcgaccctcctactttcggacagggaacaaaattggaaattaagcgtacggtggctgcacc |
| | atctgtcttcatcttcccgccatctgatgagcagttgaaatctggaactgcctctgttgtgtgcctgctgaataacttcta |
| | tcccagagaggccaaagtacagtggaaggtggataacgccctccaatcgggtaactcccaggagagtgtcacagagcagga |
| | cagcaaggacagcacctacagcctcagcagcaccctgacgctgagcaaagcagactacgagaaacacaaagtctacg |
| | cctgcgaagtcacccatcagggcctgagctcgcccgtcacaaagagcttcaacaggggagagtgttag(SEQ ID |
| | NO: 169) |
| huCD37- | gaattcgccaccatggggtggtcctgtattatcctgttcctggtcgcaaccgccacaggcgttcactccgagatcgtgttga |
| 57 | ctcagagcccagccaccatgtccgcttcccccggggagagagtgacaatgacttgttccgccacaagttctgtaacctac |
| | atgcattggtaccagcaaaaaccaggacagagtccccgtcgttggatttatgatacctctaacctggcttcaggcgttcctg |
| | cccgcttttctggtagtggatctgggacttcctatagccttaccataagctctatggaagccgaggacgccgctacatacta |
| | ctgccagcagtggagtgataacccccccaccttcgggcagggaaccaaattggagatcaaacgtacggtggctgcacc |
| | atctgtcttcatcttcccgccatctgatgagcagttgaaatctggaactgcctctgttgtgtgcctgctgaataacttct |
| | atcccagagaggccaaagtacagtggaaggtggataacgccctccaatcgggtaactcccaggagagtgtcacagagcagga |
| | cagcaaggacagcacctacagcctcagcagcaccctgacgctgagcaaagcagactacgagaaacacaaagtctacg |
| | cctgcgaagtcacccatcagggcctgagctcgcccgtcacaaagagcttcaacaggggagagtgttag(SEQ ID |
| | NO: 170) |

What is claimed is:

1. A method for increasing the efficacy of cancer therapy with an anti-CD37 antibody, or anti-CD37 immunoconjugate, said method comprising administering to a subject having cancer an anti-CD37 antibody, or anti-CD37 immunoconjugate, wherein increased expression of CD37 gene or protein in a cancerous sample from said subject has been detected using a detection method that distinguishes between staining intensity or staining uniformity in a CD37 expressing cancerous sample as compared to staining intensity or staining uniformity in one or more reference samples.

2. A method of identifying a cancer as sensitive to treatment with an anti-CD37 antibody, or anti-CD37 immunoconjugate, said method comprising:
(a) measuring the level of CD37 expression in a cancerous sample obtained from said cancer, wherein said measuring comprises the use of a detection method that distinguishes between staining intensity or staining uniformity in a CD37 expressing cancerous sample as compared to staining intensity or staining uniformity in one or more reference samples;
(b) determining a CD37 staining intensity or staining uniformity score for said cancerous sample; and
(c) comparing the CD37 staining intensity or staining uniformity score determined in step (b) to a relative value determined by measuring CD37 protein expression in at least one reference sample, wherein said at least one reference sample is a tissue, cell, or cell pellet sample which is not sensitive to treatment with an anti-CD37 antibody, or anti-CD37 immunoconjugate, and wherein a CD37 staining intensity score for said sample determined in step (b) that is higher than said relative value identifies said cancer as being sensitive to treatment with an anti-CD37 antibody, or anti-CD37 immunoconjugate.

3. A method for identifying a cancer likely to respond to an anti-CD37 antibody, or anti-CD37 immunoconjugate, said method comprising:
(a) contacting a biological sample comprising cells from said cancer with an agent that binds CD37 protein on the cell surface;
(b) detecting binding of said agent that binds CD37 protein on the cell surface of said biological sample of (a);
(c) assigning a score to said binding of step (b), wherein said score is assigned based on comparison to one or more reference samples; and
(d) comparing said score in step (c) to the score of a reference tissue or cell, wherein a score for said cancer CD37 level that is greater than the score for a negative or low CD37 expressing reference sample or a score for said cancer CD37 level that is equal to or greater than the score for a high CD37 expressing reference sample identifies said cancer as likely to respond to an anti-CD37 antibody or anti-CD37 immunoconjugate.

4. A method of identifying a subject having a cancer as likely to respond to a low dose anti-CD37 antibody or anti-CD37 immunoconjugate treatment regimen, said method comprising:
(a) contacting a biological sample comprising cells from said cancer with an agent that binds cell surface CD37 protein;
(b) detecting binding of said agent to said biological sample of (a);
(c) assigning a score to said binding of step (b), wherein said score is assigned based on comparison to one or more reference samples; and
(d) comparing said score in step (c) to the score of a reference tissue or cell, wherein a score for said cancer CD37 level that is greater than the score for a negative or low CD37 expressing reference sample or a score for said cancer CD37 level that is equal to or greater than the score for a high CD37 expressing reference sample identifies said cancer as likely to respond to a low dose anti-CD37 antibody or anti-CD37 immunoconjugate.

5. A method of optimizing a therapeutic regimen with an anti-CD37 antibody or an anti-CD37 immunoconjugate for a subject having cancer, said method comprising:
(a) detecting the level of CD37 expression in a cancerous sample obtained from said subject;
(b) comparing the level of CD37 expression in the cancerous sample to the CD37 expression in a reference sample;
(c) determining a CD37 staining intensity score for said cancerous sample; and
(d) administering an increased dose of an anti-CD37 antibody or an anti-CD37 immunoconjugate to the subject if the score is low or administering a decreased dose of an anti-CD37 antibody or an anti-CD37 immunoconjugate to the subject if the score is high.

6. A method of detecting the expression of cell surface CD37 on cancer cells in a cancerous sample from a subject, said method comprising:
(a) obtaining a cancerous sample, wherein said sample is formalin-fixed paraffin embedded;
(b) contacting said sample with an antibody that specifically binds cell surface CD37;
(c) measuring the binding of said antibody in (b) to said cell surface CD37 in said cancerous sample using a detection method that can distinguish between staining intensity or staining uniformity in a CD37 expressing sample as compared to staining intensity or staining uniformity in one or more reference samples; and
(d) assigning a CD37 expression score to said CD37 after comparing the level of cell surface CD37 staining intensity or staining uniformity in said tumor cancerous sample to one or more reference samples.

7. The method of any one of claims 1-6, wherein the detection is by immunohistochemistry (IHC).

8. The method of claim 7, wherein said IHC is calibrated IHC that can distinguish different levels of CD37 expression.

9. The method of any one of claims 1-8, wherein said detection method produces a range of staining intensity for samples having low cell surface CD37 expression, intermediate CD37 cell surface expression, or high CD37 cell surface expression.

10. The method of any one of 1-9, wherein said detection method distinguishes between staining intensity and staining uniformity in a CD37 expressing cancerous sample as compared to a reference sample.

11. The method of any one of claims 1-10, wherein the cancerous sample or biological sample has a staining intensity score of 2, 3, or 3+ for CD37 expression by immunohistochemistry.

12. The method of claim 11, wherein the cancerous sample or biological sample has a staining intensity score of 2, 3, or 3+ for CD37 expression by immunohistochemistry on a formalin fixed paraffin embedded sample.

13. The method of any of claims 10-12, wherein the cancerous sample or biological sample has a staining uniformity for CD37 expression that is homogenous.

14. The method of any one of claims 7-12, wherein the cancerous sample or biological sample has a staining intensity score of 2, 3, or 3+ for CD37 and a staining uniformity that is heterogeneous or homogenous.

15. The method of any one of claims 7-14, wherein the immunohistochemistry is performed manually.

16. The method of any one of claims 7-14, wherein the immunohistochemistry is performed using an automated system.

17. The method of any one of claims 1-16, wherein said reference sample is a positive reference sample or a negative reference sample.

18. The method of any one of claims 1-17, wherein the reference sample comprises cells, cell pellets, or tissue.

19. The method of any one of claims 1-18, wherein the detection comprises detecting CD37 expression with an antibody that specifically binds cell surface CD37.

20. The method of claim 19, wherein said antibody is CT1.

21. The method of claim 19 or 20, wherein said antibody further comprises a detection reagent selected from the group consisting of an enzyme, a fluorophore, a radioactive label, and a luminophore.

22. The method of claim 21, wherein said detection reagent is selected from the group consisting of biotin, digoxigenin, fluorescein, tritium, rhodamine, and horseradish peroxidase.

23. The method of any one of claims 19-22, wherein the concentration of said antibody is about 1-10 μg/mL.

24. The method of claim 23, wherein the concentration of said antibody is about 4-5 μg/mL.

25. The method of claim 24, wherein the concentration of said antibody is about 4.2 μg/mL

26. The method of any one of claims 1-25, wherein said cancer is selected from the group consisting of B cell lymphomas, NHL, precursor B cell lymphoblastic leukemia/lymphoma and mature B cell neoplasms, B cell chronic lymphocytic leukemia (CLL)/small lymphocytic lymphoma (SLL), B cell prolymphocytic leukemia, lymphoplasmacytic lymphoma, mantle cell lymphoma (MCL), follicular lymphoma (FL), low grade, intermediate-grade and high-grade (FL), cutaneous follicle center lymphoma, marginal zone B cell lymphoma, MALT type marginal zone B cell lymphoma, nodal marginal zone B cell lymphoma, splenic type marginal zone B cell lymphoma, hairy cell leukemia, diffuse large B cell lymphoma (DLBCL), Burkitt's lymphoma (BL), plasmacytoma, plasma cell myeloma, post-transplant lymphoproliferative disorder, Waldenstrom's macroglobulinemia, and anaplastic large-cell lymphoma (ALCL).

27. The method of any one of claims 1-26, wherein said cancerous sample or said biological sample is tissue, blood, plasma, bone marrow or lymph.

28. An article of manufacture comprising an anti-CD37 antibody or an anti-CD37 immunoconjugate, a container, and a package insert or label indicating that the antibody or immunoconjugate can be used to treat a cancer characterized by the expression of CD37 at a level of 2, 3, or 3+ measured by IHC.

29. A combination diagnostic and pharmaceutical kit comprising a murine anti-CD37 antibody for use in diagnosis and an anti-CD37 antibody or an anti-CD37 immunoconjugate for use in therapy.

30. The combination diagnostic and pharmaceutical kit of claim 29, wherein the diagnostic antibody is a detection antibody that is able to detect CD37 expression by IHC.

31. A diagnostic kit comprising a detection antibody that specifically binds to cell surface CD37, a reagent for immunohistochemistry (IHC), and one or more standardized reference samples, wherein said standardized reference samples comprise cells, cell pellets, or formalin fixed paraffin embedded tissue samples, and wherein said one or more standardized referenced samples are from non-CD37 expressing, low-CD37 expressing, or high CD37 expressing cells, cell pellets, or tissues.

32. The article of manufacture of claim 28 or the kit of claim 30 or 31, wherein said IHC is calibrated IHC that can distinguish different levels of CD37 expression.

33. The article of manufacture or kit of claim 32, wherein said calibrated IHC produces a range of staining intensity for samples having low cell surface CD37 expression, intermediate cell surface CD37 expression, or high cell surface CD37 expression.

34. The article of manufacture or the kit of any one of claim 28 or 30-33, wherein said IHC distinguishes between staining intensity and staining uniformity in a CD37 expressing sample as compared to a reference sample.

35. The article of manufacture or kit of any one of claim 28 or 30-33, wherein said IHC is performed on a formalin fixed paraffin embedded sample.

36. The article of manufacture or kit of any one of claim 28 or 30-33, wherein said IHC is performed manually.

37. The article of manufacture or kit of any one of claim 28 or 30-33, wherein said IHC is performed using an automated system.

38. The article of manufacture or kit of any one of claim 28-30 or 32-37, wherein the CD37 immunoconjugate comprises an anti-CD37 antibody, a linker, and a cytotoxin.

39. The article of manufacture or kit of claim 38, wherein the anti-CD37 antibody is chimeric, or humanized CD37-3, CD37-38, or CD37-50.

40. The article of manufacture or kit of claim 38 or 39, wherein said linker is selected from the group consisting of a cleavable linker, a non-cleavable linker, a hydrophilic linker, and a dicarboxylic acid based linker.

41. The article of manufacture or kit of claim 40, wherein said linker is selected from the group consisting: N-succinimidyl 4-(2-pyridyldithio)pentanoate (SPP) or N-succinimidyl 4-(2-pyridyldithio)-2-sulfopentanoate (sulfo-SPP); N-succinimidyl 4-(2-pyridyldithio)butanoate (SPDB) or N-succinimidyl 4-(2-pyridyldithio)-2-sulfobutanoate (sulfo-SPDB); N-succinimidyl 4-(maleimidomethyl) cyclohexanecarboxylate (SMCC); N-sulfosuccinimidyl 4-(maleimidomethyl) cyclohexanecarboxylate (sulfoSMCC); N-succinimidyl-4-(iodoacetyl)-aminobenzoate (SIAB); and N-succinimidyl-[(N-maleimidopropionamido)-tetraethyleneglycol]ester (NHS-PEG4-maleimide).

42. The article of manufacture or kit of claim 41, wherein said linker is N-succinimidyl 4-(maleimidomethyl) cyclohexanecarboxylate (SMCC).

43. The article of manufacture or kit of any one of claims 38-42, wherein said cytotoxin is selected from the group consisting of a maytansinoid, maytansinoid analog, benzodiazepine, taxoid, CC-1065, CC-1065 analog, duocarmycin, duocarmycin analog, calicheamicin, dolastatin, dolastatin analog, auristatin, tomaymycin derivative, and leptomycin derivative or a prodrug of the cytotoxin.

44. The article of manufacture or kit of claim 43, wherein said cytotoxin is a maytansinoid.

45. The article of manufacture or kit of claim 44, wherein said maytansinoid is N(2′)-deacetyl-N(2′)-(3-mercapto-1-oxopropyl)-maytansine or N(2′)-deacetyl-N2-(4-mercapto-4-methyl-1-oxopentyl)-maytansine.

46. The article of manufacture or kit of claim 45, wherein said maytansinoid is N(2′)-deacetyl-N(2′)-(3-mercapto-1-oxopropyl)-maytansine (DM1).

47. The article of manufacture or kit of any one of claims 38-46, wherein said immunoconjugate comprises the antibody huCD37-3, the linker SMCC, and the maytansinoid DM1.

48. The combination diagnostic and pharmaceutical kit of any one of claims 29-30 and 32-47, further comprising one or more reference samples.

49. The combination diagnostic and pharmaceutical kit of claim 48, wherein said reference sample is a positive reference sample or a negative reference sample.

50. The combination diagnostic and pharmaceutical kit of claim 48 or 49, wherein the reference sample comprises cells, cell pellets, or tissue.

51. The kit of any one of claims 30-37, wherein said detection antibody further comprises a detection reagent selected from the group consisting of: an enzyme, a fluorophore, a radioactive label, and a luminophore.

52. The kit of claim 51, wherein said detection reagent is selected from the group consisting of: biotin, digoxigenin, fluorescein, tritium, rhodamine, and horseradish peroxidase.

53. The kit of claim any one of claim 30-37 or 51-52, wherein the concentration of the detection antibody is about 1-10 μg/mL.

54. The kit of claim 53, wherein the concentration of the detection antibody is about 4-5 μg/mL.

55. The kit of claim 54, wherein the concentration of the detection antibody is about 4.2 μg/mL.

56. The kit of claim 31, wherein the low-CD37 expressing control is Namalwa or RL tumor cells.

57. The kit of claim 31, wherein the high-CD37 expressing control is selected from the group consisting of: Daudi and Ramos cell lines, and a cell line stably or transiently transfected with CD37.

58. The kit of claim 57, wherein said cell line stably or transiently transfected with CD37 is 300-19/CD37.
